SEQUENCE LISTING (29 sequences)

SEQ ID NO: 1 - protein, 15 aa, Artificial Sequence
  synthetic peptide
MVRIKPGSANKPSDD

SEQ ID NO: 2 - protein, 15 aa, Artificial Sequence
  synthetic peptide
MVRIKYGSYNNKPSD

SEQ ID NO: 3 - protein, 13 aa, Artificial Sequence
  synthetic peptide
MVRIKPGSANKPS

SEQ ID NO: 4 - protein, 9 aa, Artificial Sequence
  synthetic peptide
MVRIKPGSA

SEQ ID NO: 5 - protein, 15 aa, Artificial Sequence
  synthetic peptide
GRVDPSNKSMPKDIA

SEQ ID NO: 6 - protein, 10 aa, Artificial Sequence
  synthetic peptide
SQAVQEHAST

SEQ ID NO: 7 - protein, 13 aa, Artificial Sequence
  synthetic peptide
SQAVQEHASTNMG

SEQ ID NO: 8 - protein, 13 aa, Artificial Sequence
  synthetic peptide
EVAQHSQASTMNG

SEQ ID NO: 9 - protein, 14 aa, Artificial Sequence
  synthetic peptide
GQASSAQMNSTGVH

SEQ ID NO: 10 - protein, 18 aa, Artificial Sequence
  synthetic peptide
SGIVIYQYMDDRYVGSDL

SEQ ID NO: 11 - protein, 11 aa, Artificial Sequence
  synthetic peptide
GMRELQRSANK

SEQ ID NO: 12 - protein, 9 aa, Artificial Sequence
  synthetic peptide
VIIIAQYMD

SEQ ID NO: 13 - protein, 15 aa, Artificial Sequence
  synthetic peptide
MVRIKPGSANKPSDD

SEQ ID NO: 14 - protein, 13 aa, Artificial Sequence
  synthetic peptide
SQAVQEHASTNMG

SEQ ID NO: 15 - protein, 18 aa, Artificial Sequence
  synthetic peptide
SGIVIYQYMDDRYVGSDL

SEQ ID NO: 16 - protein, 9 aa, Artificial Sequence
  synthetic peptide
MVRIKPGSA

SEQ ID NO: 17 - protein, 9 aa, Artificial Sequence
  synthetic peptide
VIIIAQYMD

SEQ ID NO: 18 - protein, 5 aa, Artificial Sequence
  synthetic peptide
MVRIK

SEQ ID NO: 19 - protein, 4 aa, Artificial Sequence
  synthetic peptide
PGSA

SEQ ID NO: 20 - protein, 15 aa, Artificial Sequence
  synthetic peptide
  Xaa (3)..(3): Xaa is any amino acid except arginine
  MISC_FEATURE (3)..(3): Xaa is any amino acid except arginine.
MVXIKPGSANKPSDD

SEQ ID NO: 21 - protein, 15 aa, Artificial Sequence
  synthetic peptide
  MISC_FEATURE (1)..(1): Xaa is any amino acid except arginine.
XVRIKPGSANKPSDD

SEQ ID NO: 22 - protein, 15 aa, Artificial Sequence
  synthetic peptide
  MISC_FEATURE (1)..(1): Xaa is any amino acid except arginine
  MISC_FEATURE (3)..(3): Xaa is any amino acid except arginine
XVXIKPGSANKPSDD

SEQ ID NO: 23 - protein, 15 aa, Artificial Sequence
  synthetic peptide
  MISC_FEATURE (6)..(6): Xaa is any amino acid except arginine
MVRIKXGSANKPSDD

SEQ ID NO: 24 - protein, 15 aa, Artificial Sequence
  synthetic peptide
  MISC_FEATURE (4)..(4): Xaa is any amino acid except arginine
MVRXKPGSANKPSDD

SEQ ID NO: 25 - protein, 15 aa, Artificial Sequence
  synthetic peptide
  MISC_FEATURE (2)..(2): Xaa is any amino acid except arginine
MXRIKPGSANKPSDD

SEQ ID NO: 26 - protein, 15 aa, Artificial Sequence
  synthetic peptide
MVRIKEGSANKPSDD

SEQ ID NO: 27 - protein, 15 aa, Artificial Sequence
  synthetic peptide
MVRGKPGSANKPSDD

SEQ ID NO: 28 - protein, 15 aa, Artificial Sequence
  synthetic peptide
MERIKPGSANKPSDD

SEQ ID NO: 29 - protein, 15 aa, Artificial Sequence
  synthetic peptide
AVRIKPGSANKPSDD

(19) United States
(12) Patent Application Publication
(10) Pub. No.: US 2017/0336406 A1
(43) Pub. Date: Nov. 23, 2017

(54) PIF BINDING AS A MARKER FOR IMMUNE DYSREGULATION

(71) Applicant: BioIncept, LLC, Cherry Hill, NJ (US)

(72) Inventor: Eytan R. Barnea, Cherry Hill, NJ (US)

(73) Assignee: BioIncept, LLC, Cherry Hill, NJ (US)

(21) Appl. No.: 15/524,249

(22) PCT Filed: Nov. 3, 2015
(86) PCT No.: PCT/US15/58877

§ 371 Date: May 3, 2017

Related U.S. Application Data
(63) Continuation of application No. PCT/US15/50532, filed on Sep. 16, 2015.
(60) Provisional application No. 62/074,384, filed on Nov. 3, 2014.
(60) Provisional application No. 62/113,298, filed on Feb. 6, 2015.
(60) Provisional application No. 62/211,660, filed on Aug. 28, 2015.
(60) Provisional application No. 62/051,077, filed on Sep. 16, 2014.

(51) Int. Cl. G01N 33/564; G01N 33/569; G01N 33/68; G01N 33/567; C07K 7/08

(52) U.S. Cl. CPC G01N 33/564; G01N 33/6863; G01N 33/567; G01N 33/56972; G01N 33/689; C07K 7/08; G01N 2800/364; G01N 2800/368

(57) ABSTRACT

Embodiments are directed to methods of examining preimplantation factor (PIF) binding to a subject's circulating immune cells as a marker for immune dysregulation. Some embodiments are directed to methods of detecting a level of immune dysregulation sufficient to cause recurrent pregnancy loss (RPL), methods of detecting a level of immune dysfunction sufficient to cause endometriosis, and methods of detecting a level of immune dysfunction comprising administering an effective amount of PIF or an analog thereof, and examining its binding to circulating immune cells. Within those methods, an about twenty percent change in PIF binding to a subject's circulating immune cells indicates a level of immune dysfunction.

### CROSS-REFERENCE TO RELATED APPLICATIONS

This application claims priority to and benefit of U.S. Provisional Application Ser. No. 62/074,384 filed Nov. 3, 2014, entitled “PIF Binding as a Marker for Immune Dysregulation,” U.S. Provisional Application Ser. No. 62/113,298 filed Feb. 6, 2015, entitled “PIF Binding as a Marker for Immune Dysregulation,” U.S. Provisional Application Ser. No. 62/211,660 filed Aug. 28, 2015, entitled “Compositions and Methods for the Treatment of Neurodamage,” and International Application Serial No. PCT/US15/50532 filed Sep. 16, 2015, entitled “Compositions and Methods for Treating Acute Radiation Syndrome,” which claims priority to and benefit of U.S. Provisional Application Ser. No. 62/051,077 filed Sep. 16, 2014, entitled “Methods for Treating Acute Radiation Syndrome.” The contents of each application are incorporated herein by reference in their respective entireties.

### FIELD OF THE DISCLOSURE

The present disclosure relates generally to diagnostic applications directed to the identification of immune dysregulation in a subject by detection and/or quantification of PIF binding to cells or other biological samples from the subject. The present disclosure is also directed to the diagnosis of recurrent pregnancy loss and endometriosis caused by immune dysregulation from analysis of samples obtained from animals including humans. The identification of immune dysregulation is important for determining a proper course of treatment and/or eradication of the diseases caused by immune dysregulation.

### BACKGROUND

Recurrent pregnancy loss (RPL), also referred to as recurrent miscarriage or habitual abortion, is historically defined as 3 consecutive pregnancy losses prior to 20 weeks from the last menstrual period. Based on the incidence of sporadic pregnancy loss, the incidence of recurrent pregnancy loss should be approximately 1 in 300 pregnancies. However, epidemiologic studies have revealed that 1% to 2% of women experience recurrent pregnancy loss. Defining RPL as a clinical entity requiring diagnostic testing and therapeutic intervention rests on knowledge of the elevation of risk for subsequent fetal loss and the probability of finding a treatable etiology for the disorder. Although no reliable published data have estimated the probability of finding an etiology for RPL in a population with 2 versus 3 or more miscarriages, the best available data suggest that the risk of miscarriage in subsequent pregnancies is 30% after 2 losses, compared with 33% after 3 losses among patients without a history of a live birth. Approximately a third or more of all cases of RPL will remain unexplained.

Endometriosis is histologically characterized by the displacement of endometrial tissue to extrauterine locations including the pelvic peritoneum, ovaries, and bowel. An important cause of infertility and pelvic pain, the individual and global socioeconomic burden of endometriosis is significant. Laparoscopy remains the gold standard for the diagnosis of the condition. However, the invasive nature of surgery, coupled with the lack of a laboratory biomarker for the disease, results in a mean latency of 7-11 years from onset of symptoms to definitive diagnosis. Unfortunately, the delay in diagnosis may have significant consequences in terms of disease progression.

Mammalian pregnancy is a unique physiological event in which the maternal immune system interacts with the fetus in a very efficient manner that is beneficial for both parties. The embryo-derived factor preimplantation factor (PIF-1) may cause immune tolerance of pregnancy by creating maternal recognition of pregnancy shortly after fertilization.

### SUMMARY OF EMBODIMENTS

The PIF binding profile to cellular receptors on immune cells can be exploited to create a system or device useful to diagnose immune dysregulation in a subject. The disclosure relates to a solid support comprising immobilized PIF, where PIF binding affinity to a sample may be analyzed to identify a patient population suffering from recurrent pregnancy loss and/or endometriosis due to immune dysregulation.

The disclosure relates to methods of examining preimplantation factor (PIF) or a functional fragment thereof or analogs thereof binding to a subject's circulating immune cells as a marker for immune dysregulation. Some embodiments are directed to a method of identifying a female subject with a history of recurrent pregnancy loss (RPL) due to immune dysregulation comprising exposing an effective amount of PIF or a functional fragment thereof to a sample from the subject comprising one or a plurality of immune cells, and examining a binding event between the one or among a plurality of immune cells of the subject and PIF or a functional fragment thereof, wherein a significant change of binding of PIF to the one or plurality of immune cells as compared to a reference indicates that said RPL is due to immune dysregulation.

In some embodiments, an insignificant change of binding of PIF to the one or plurality of the functional fragments thereof to the one or plurality of immune cells as compared to a reference indicates that the RPL is not due to immune dysregulation.

In some embodiments, the effective amount of PIF may be from about 300 nM to about 500 nM PIF in solution or immobilized by an antibody bound adsorbed or ligated to a matrix material coated on a plastic surface.

The disclosure provides embodiments in which a method may further comprise isolating a sample from the subject prior to exposing the sample to PIF or a functional fragment thereof. In some embodiments, the method may further comprise immobilizing PIF or a functional fragment thereof or an analog thereof to a solid support prior to exposing the PIF or function fragment thereof or an analog thereof to one or a plurality of immune cells, wherein the solid support is chosen from a chip, a column, a plate, or a multiwell plate.

In some embodiments, the step of examining a binding event may comprise observing, quantifying and/or detecting the association between PIF or a function fragment thereof and one or a plurality of immune cells. In some embodiments, the step of examining a binding event may comprise observing, quantifying and/or detecting an amount of expression of one or a plurality of cytokines by the one or plurality of immune cells. In some embodiments, the step of examining a binding event may comprise observing, quantifying, and/or detecting a number of immune cells that bind to PIF or a functional fragment thereof, wherein the one or more immune cells may comprise one or a combination of CD3+ cells, CD4+ cells, CD14+ cells, CD45+ cells, dendritic cells, or peripheral blood mononuclear cells (PBMCs).

In some embodiments, the step of examining a binding event may comprise quantifying the number of immune cells in a sample by flow cytometry.

In some embodiments, the PIF or a functional fragment thereof may be immobilized to a column prior to exposing the PIF or functional fragment thereof to the one or plurality of immune cells, wherein the step of exposing the PIF or functional fragment thereof to the one or plurality of immune cells comprises exposing sample of one or a plurality of immune cells to the column comprising immobilized PIF or a functional fragment thereof, and wherein the step of examining a binding event comprises quantifying a number of one or a plurality of immune cells by flow cytometry, wherein the one or plurality of immune cells comprise one or a combination of CD3+ cells, CD4+ cells, CD14+ cells, CD45+ cells, dendritic cells, or PBMCs.

In some embodiments, the significant change may comprise quantifying a decrease in said PIF binding to CD 14+ and/or dendritic cells.

In some embodiments, the significant change may comprise quantifying an increase in said PIF binding to CD4+, CD8+, and/or natural killer (NK.) cells. In some embodiments, the PIF or functional fragment thereof or analog thereof comprises one or more fluorescein isothiocyanate (FITC) labels, and wherein a binding event is measured by quantifying and/or detecting the level of fluorescence in a sample exposed to a FITC-labeled PIF or analog thereof after stimulation of the sample with a wavelength of light sufficient to cause fluorescence of the FITC.

The methods of the disclosure relate to a step of exposing PIF or a functional fragment thereof or an analog thereof to one or a plurality of immune cells of a subject. In some embodiments the may comprise administering the PIF or a functional fragment thereof or an analog thereof to a subject. In some embodiments, the significant change may comprise one or a combination of a reduction of PIF or a functional fragment thereof binding to dendritic cells, an increase of PIF or a functional fragment thereof binding to CD 14+ cells, and an increase of PIF binding to CD4+ cells.

Some embodiments are directed to a method of identifying a female subject likely to suffer from RPL due to immune dysregulation comprising exposing an effective amount of PIF or a functional fragment thereof to a sample from the subject comprising one or a plurality of immune cells, and examining a binding event between the one or among a plurality of immune cells of the subject and PIF or a functional fragment thereof, wherein a significant change of binding of PIF to the one or plurality of immune cells as compared to a reference indicates that said female subject is likely to suffer from RPL due to immune dysregulation.

In some embodiments, an insignificant change of binding of PIF to the one or plurality of the functional fragments thereof to the one or plurality of immune cells as compared to a reference indicates that said female subject is not likely to suffer from RPL due to immune dysregulation.

In some embodiments, the effective amount of PIF may be from about 300 nM to about 500 nM PIF.

In some embodiments, the method may further comprise isolating a sample from the subject prior to exposing the sample to PIF or a functional fragment thereof.

In some embodiments, the method may further comprise immobilizing PIF or a functional fragment thereof to a solid support prior to exposing the PIF or function fragment thereof to one or a plurality of immune cells, wherein the solid support is chosen from a chip, a column, a plate, or a multiwell plate.

In some embodiments, the step of examining a binding event may comprise observing, quantifying and/or detecting the association between PIF or a function fragment thereof and one or a plurality of immune cells.

In some embodiments, the step of examining a binding event may comprise observing, quantifying and/or detecting an amount of expression of one or a plurality of cytokines by the one or plurality of immune cells.

In some embodiments, the step of examining a binding event may comprise observing, quantifying, and/or detecting a number of immune cells that bind to PIF or a functional fragment thereof, wherein the one or more immune cells may comprise one or a combination of CD3+ cells, CD4+ cells, CD14+ cells, CD45+ cells, dendritic cells, or peripheral blood mononuclear cells (PBMCs).

In some embodiments, the step of examining a binding event may comprise quantifying the number of immune cells by flow cytometry.

In some embodiments, the PIF or a functional fragment thereof may be immobilized to a column prior to exposing the PIF or functional fragment thereof to the one or plurality of immune cells, wherein the step of exposing the PIF or functional fragment thereof to the one or plurality of immune cells comprises exposing sample of one or a plurality of immune cells to the column comprising immobilized PIF or a functional fragment thereof, and wherein the step of examining a binding event comprises quantifying a number of one or a plurality of immune cells by flow cytometry, wherein the one or plurality of immune cells comprise one or a combination of CD3+ cells, CD4+ cells, CD14+ cells, CD45+ cells, dendritic cells, or PBMCs.

In some embodiments, the significant change may comprise quantifying a decrease in said PIF binding to CD14+ and/or dendritic cells.

In some embodiments, the significant change may comprise quantifying an increase in said PIF binding to CD4+, CD8+, and/or natural killer (NK) cells.

In some embodiments, the PIF or functional fragment thereof comprises one or more fluorescein isothiocyanate (FITC) labels, and wherein a binding event is measured by quantifying and/or detecting the level of fluorescence.

In some embodiments, the step of exposing PIF or a functional fragment thereof to one or a plurality of immune cells may comprise administering the PIF or a functional fragment thereof to the subject.

In some embodiments, the significant change may comprise one or a combination of a reduction of PIF or a functional fragment thereof binding to dendritic cells, an increase of PIF or a functional fragment thereof binding to CD14+ cells, and an increase of PIF binding to CD4+ cells.

Some embodiments are directed to a method of identifying a female subject with endometriosis comprising exposing an effective amount of PIF or a functional fragment thereof to a sample from the subject comprising one or a plurality of immune cells, and examining a binding event between the one or among a plurality of immune cells of the subject and PIF or a functional fragment thereof, wherein a significant change of binding of PIF to the one or plurality of immune cells as compared to a reference indicates that said female subject has endometriosis.

In some embodiments, an insignificant change of binding of PIF to the one or plurality of the functional fragments thereof to the one or plurality of immune cells as compared to a reference indicates that the female subject does not have endometriosis.

In some embodiments, the effective amount of PIF may be from about 300 nM to about 500 nM PIF.

In some embodiments, the method may further comprise isolating a sample from the subject prior to exposing the sample to PIF or a functional fragment thereof.

In some embodiments, the method may further comprise immobilizing PIF or a functional fragment thereof to a solid support prior to exposing the PIF or function fragment thereof to one or a plurality of immune cells, wherein the solid support is chosen from a chip, a column, a plate, or a multiwell plate. In some embodiments, the step of examining a binding event may comprise observing, quantifying and/or detecting the association between PIF or a function fragment thereof and one or a plurality of immune cells.

In some embodiments, the step of examining a binding event may comprise observing, quantifying and/or detecting an amount of expression of one or a plurality of cytokines by the one or plurality of immune cells.

In some embodiments, the step of examining a binding event may comprise observing, quantifying, and/or detecting a number of immune cells that bind to PIF or a functional fragment thereof, wherein the one or more immune cells may comprise one or a combination of CD3+ cells, CD4+ cells, CD14+ cells, CD45+ cells, dendritic cells, or peripheral blood mononuclear cells (PBMCs).

In some embodiments, the step of examining a binding event may comprise quantifying the number of immune cells by flow cytometry.

In some embodiments, the PIF or a functional fragment thereof may be immobilized to a column prior to exposing the PIF or functional fragment thereof to the one or plurality of immune cells, wherein the step of exposing the PIF or functional fragment thereof to the one or plurality of immune cells comprises exposing sample of one or a plurality of immune cells to the column comprising immobilized PIF or a functional fragment thereof, and wherein the step of examining a binding event comprises quantifying a number of one or a plurality of immune cells by flow cytometry, wherein the one or plurality of immune cells comprise one or a combination of CD3+ cells, CD4+ cells, CD14+ cells, CD45+ cells, dendritic cells, or PBMCs.

In some embodiments, the significant change may comprise quantifying a decrease in said PIF binding to CD 14+ and/or dendritic cells.

In some embodiments, the significant change may comprise quantifying an increase in said PIF binding to CD4+, CD8+, and/or natural killer (NK) cells.

In some embodiments, the PIF or functional fragment thereof comprises one or more fluorescein isothiocyanate (FITC) labels, and wherein a binding event is measured by quantifying and/or detecting the level of fluorescence.

In some embodiments, the step of exposing PIF or a functional fragment thereof to one or a plurality of immune cells may comprise administering the PIF or a functional fragment thereof to the subject.

In some embodiments, the significant change may comprise one or a combination of a reduction of PIF or a functional fragment thereof binding to dendritic cells, an increase of PIF or a functional fragment thereof binding to CD14+ cells, and an increase of PIF binding to CD4+ cells.

Some embodiments are directed to a method of identifying a female subject likely to suffer from endometriosis due to immune dysregulation comprising exposing an effective amount of PIF or a functional fragment thereof to a sample from the subject comprising one or a plurality of immune cells, and examining a binding event between the one or among a plurality of immune cells of the subject and PIF or a functional fragment thereof, wherein a significant change of binding of PIF to the one or plurality of immune cells as compared to a reference indicates that said female subject is likely to suffer from endometriosis due to immune dysregulation.

In some embodiments, an insignificant change of binding of PIF to the one or plurality of the functional fragments thereof to the one or plurality of immune cells as compared to a reference indicates that the female subject is not likely to suffer from endometriosis due to immune dysregulation.

In some embodiments, the effective amount of PIF may be from about 300 nM to about 500 nM PIF.

In some embodiments, the method may further comprise isolating a sample from the subject prior to exposing the sample to PIF or a functional fragment thereof.

In some embodiments, the method may further comprise immobilizing PIF or a functional fragment thereof to a solid support prior to exposing the PIF or function fragment thereof to one or a plurality of immune cells, wherein the solid support is chosen from a chip, a column, a plate, or a multiwell plate.

In some embodiments, the step of examining a binding event may comprise observing, quantifying and/or detecting the association between PIF or a function fragment thereof and one or a plurality of immune cells.

In some embodiments, the step of examining a binding event may comprise observing, quantifying and/or detecting an amount of expression of one or a plurality of cytokines by the one or plurality of immune cells.

In some embodiments, the step of examining a binding event may comprise observing, quantifying, and/or detecting a number of immune cells that bind to PIF or a functional fragment thereof, wherein the one or more immune cells may comprise one or a combination of CD3+ cells, CD4+ cells, CD14+ cells, CD45+ cells, dendritic cells, or peripheral blood mononuclear cells (PBMCs).

In some embodiments, the step of examining a binding event may comprise quantifying the number of immune cells by flow cytometry.

In some embodiments, the PIF or a functional fragment thereof may be immobilized to a column prior to exposing the PIF or functional fragment thereof to the one or plurality of immune cells, wherein the step of exposing the PIF or functional fragment thereof to the one or plurality of immune cells comprises exposing sample of one or a plurality of immune cells to the column comprising immobilized PIF or a functional fragment thereof, and wherein the step of examining a binding event comprises quantifying a number of one or a plurality of immune cells by flow cytometry, wherein the one or plurality of immune cells comprise one or a combination of CD3+ cells, CD4+ cells, CD 14+ cells, CD45+ cells, dendritic cells, or PBMCs.

In some embodiments, the significant change may comprise quantifying a decrease in said PIF binding to CD 14+ and/or dendritic cells.

In some embodiments, the significant change may comprise quantifying an increase in said PIF binding to CD4+, CD8+, and/or natural killer (NK) cells.

In some embodiments, the PIF or functional fragment thereof comprises one or more fluorescein isothiocyanate (FITC) labels, and wherein a binding event is measured by quantifying and/or detecting the level of fluorescence emitted by the FITC-labeled peptide in the presence of a wavelength of light sufficient to cause florescence of FITC moiety.

In some embodiments, the step of exposing PIF or a functional fragment thereof to one or a plurality of immune cells may comprise administering the PIF or a functional fragment thereof to the subject.

In some embodiments, the significant change may comprise one or a combination of a reduction of PIF or a functional fragment thereof binding to dendritic cells, an increase of PIF or a functional fragment thereof binding to CD14+ cells, and an increase of PIF binding to CD4+ cells.

One embodiment of the disclosure relates to a method of detecting a level of immune dysregulation sufficient to cause RPL comprising exposing a sample from a subject diagnosed with or suspected of having RPL to a solid support comprising PIF or a functional fragment thereof; quantifying a number of immune cells that bind to the PIF or the functional fragment thereof; comparing the number of immune cells bound to PIF or the functional fragment thereof to a number of immune cells that bind to PIF or the functional fragment thereof from a sample of subject that does not have known immune dysregulation sufficient to cause RPL; and classifying the subject as having immune dysregulation sufficient to cause RPL if the number of immune cells bound to PIF or the functional fragment thereof is from about fifteen percent to about twenty-five percent greater than the number of immune cells bound to PIF from the sample of subject that does not have known immune dysregulation sufficient to cause RPL. One embodiment of the disclosure relates to a method of detecting a level of immune dysregulation sufficient to cause RPL comprising exposing a sample from a subject diagnosed with or suspected of having RPL to a solid support comprising PIF or a functional fragment thereof; quantifying a number of immune cells that bind to the PIF or the functional fragment thereof; comparing the number of immune cells bound to PIF or the functional fragment thereof to a number of immune cells that bind to PIF or the functional fragment thereof from a sample of subject that does not have known immune dysregulation sufficient to cause RPL; and classifying the subject as having immune dysregulation sufficient to cause RPL if the number of immune cells bound to PIF or the functional fragment thereof is about twenty percent greater than the number of immune cells bound to PIF or the functional fragment thereof from the sample of subject that does not have known immune dysregulation sufficient to cause RPL.

Another embodiment of the disclosure relates to a method of detecting a level of immune dysregulation of a subject sufficient to cause RPL comprising detecting or quantifying a number of immune cells that bind to the immobilized PIF or a functional fragment thereof; creating a binding profile of the subject; comparing the number of immune cells bound to PIF or the functional fragment thereof to a number of immune cells that bind to PIF from a sample of subject that does not have known immune dysregulation sufficient to cause RPL; and classifying the subject as having immune dysregulation sufficient to cause RPL if the number of immune cells bound to PIF is about twenty percent greater than the number of immune cells bound to PIF or the functional fragment thereof from a sample of subject that does not have known immune dysregulation sufficient to cause RPL. In some embodiments, the immune cells are one or a plurality of CD4+ cells, CD8+ cells, and/or CD14+ cells.

One embodiment of the disclosure relates to a method of detecting a level of immune dysregulation of a subject sufficient to cause RPL comprising detecting or quantifying a number of immune cells that bind to the immobilized PIF or a functional fragment thereof; comparing the number of immune cells bound to PIF or the functional fragment thereof to a number of immune cells that bind to PIF or the functional fragment thereof from a sample of subject that does not have known immune dysregulation sufficient to cause RPL; and classifying the subject as having immune dysregulation sufficient to cause

RPL if the number of immune cells bound to PIF or a functional fragment thereof is about twenty percent greater than the number of immune cells bound to PIF from a sample of subject that does not have known immune dysregulation sufficient to cause RPL.

The disclosure also relates to a method of treating a subject having a level of immune dysregulation sufficient to cause RPL comprising detecting the presence, absence, or quantity of one or more of: CD4+ cells, CD8+ cells, and CD 14+ cells; diagnosing the subject as having a level of immune dysregulation sufficient to cause RPL if the number of immune cells is about twenty percent greater than the number of CD4+ cells, CD8+ cells, and CD 14+ cells; and treating the subject by administering an effective amount of an immunomodulating agent.

One embodiment of the disclosure relates to a method of detecting a level of immune dysregulation sufficient to cause endometriosis comprising exposing a sample from a subject diagnosed with or suspected of having endometriosis to a solid support comprising PIF or a functional fragment thereof; quantifying a number of immune cells that bind to the immobilized PIF or the functional fragment thereof; comparing the number of immune cells bound to PIF or the functional fragment thereof to a number of immune cells that bind to PIF from a sample of subject that does not have known immune dysregulation sufficient to cause endometriosis; and classifying the subject as having immune dysregulation sufficient to cause endometriosis if the number of immune cells bound to PIF or the functional fragment thereof is about twenty percent greater than the number of immune cells bound to PIF from the sample of subject that does not have known immune dysregulation sufficient to cause endometriosis.

One embodiment of the disclosure relates to a method of detecting a level of immune dysregulation of a subject sufficient to cause endometriosis comprising detecting or quantifying a number of immune cells that bind to the immobilized PIF or a functional fragment thereof; creating a binding profile of the subject; comparing the number of immune cells bound to PIF or the functional fragment thereof to a number of immune cells that bind to PIF from a sample of subject that does not have known immune dysregulation sufficient to cause endometriosis; and classifying the subject as having immune dysregulation sufficient to cause endometriosis if the number of immune cells bound to PIF or the functional fragment thereof is about twenty percent greater than the number of immune cells bound to PIF from a sample of subject that does not have known immune dysregulation sufficient to cause endometriosis.

One embodiment of the disclosure relates to a method of detecting a level of immune dysregulation of a subject sufficient to cause endometriosis comprising detecting or quantifying a number of immune cells that bind to the immobilized PIF or a functional fragment thereof; comparing the number of immune cells bound to PIF or the functional fragment thereof to a number of immune cells that bind to PIF from a sample of subject that does not have known immune dysregulation sufficient to cause endometriosis; and classifying the subject as having immune dysregulation sufficient to cause endometriosis if the number of immune cells bound to PIF or the functional fragment thereof is about twenty percent greater than the number of immune cells bound to PIF or a functional fragment thereof from a sample of subject that does not have known immune dysregulation sufficient to cause endometriosis.

One embodiment of the disclosure relates to a method of treating a subject having a level of immune dysregulation sufficient to cause endometriosis comprising

CD 14+ cells; diagnosing the subject as having a level of immune dysregulation sufficient to cause endometriosis if the number of immune cells is about twenty percent greater; and treating the subject by administering an effective amount of an immunomodulating agent.

One embodiment of the disclosure relates to a method of detecting a level of immune dysregulation comprising exposing a sample from a subject diagnosed with or suspected of having immune dysregulation to a solid support comprising PIF or a functional fragment thereof; quantifying a number of immune cells that bind to the immobilized PIF or the functional fragment thereof; comparing the number of immune cells bound to PIF or the functional fragment thereof to a number of immune cells that bind to PIF from a sample of subject that does not have known immune dysregulation; and classifying the subject as having immune dysregulation if the number of immune cells bound to PIF or the functional fragment thereof is about twenty percent greater than the number of immune cells bound to PIF from the sample of subject that does not have known immune dysregulation.

One embodiment of the disclosure relates to a method of detecting a level of immune dysregulation of a subject comprising detecting or quantifying a number of immune cells that bind to the immobilized PIF or a functional fragment thereof; creating a binding profile of the subject; comparing the number of immune cells bound to PIF or the functional fragment thereof to a number of immune cells that bind to PIF from a sample of subject that does not have known immune dysregulation; and classifying the subject as having immune dysregulation if the number of immune cells bound to PIF or the functional fragment thereof is about twenty percent greater than the number of immune cells bound to PIF from a sample of subject that does not have known immune dysregulation.

One embodiment of the disclosure relates to a method of detecting a level of immune dysregulation of a subject comprising detecting or quantifying a number of immune cells that bind to the immobilized PIF or a functional fragment thereof; comparing the number of immune cells bound to PIF or the functional fragment thereof to a number of immune cells that bind to PIF from a sample of subject that does not have known immune dysregulation; and classifying the subject as having immune dysregulation if the number of immune cells bound to PIF or the functional fragment thereof is about twenty percent greater than the number of immune cells bound to PIF from a sample of subject that does not have known immune dysregulation.

One embodiment of the disclosure relates to a method of treating a subject having a level of immune dysregulation comprising detecting the presence, absence, or quantity of one or more of: CD4+ cells, CD8+ cells, and CD14+ cells; diagnosing the subject as having a level of immune dysregulation if the number of immune cells is about twenty percent greater; and treating the subject by administering an effective amount of an immunomodulating agent.

In some embodiments, the step of quantifying comprises creating a binding profile of the subject. In some embodiments, creating a binding profile of the subject comprises correlating a level of immune dysregulation with the quantity of one or a combination of the number of CD 14+ cells bound to PIF or the functional fragment thereof, the number of CD4+ cells bound to PIF or the functional fragment thereof, and the number of CD8+ cells bound to PIF or the functional fragment thereof. In some embodiments, the methods further comprise correlating a level of immune dysregulation with the quantity of one or a combination of the number of CD 14+ cells bound to PIF or the functional fragment thereof, the number of CD4+ cells bound to PIF or the functional fragment thereof, and the number of CD8+ cells bound to PIF or the functional fragment thereof. In some embodiments, the step of correlating a level of immune dysregulation with the quantity of one or a combination of: the number of CD14+ cells bound to PIF or the functional fragment thereof, the number of CD4+ cells bound to PIF or the functional fragment thereof, and the number of CD8+ cells bound to PIF comprises detecting and/or quantifying binding association of PIF or a functional fragment to the cells. In some embodiments, the method further comprises correlating the binding association of PIF or a functional fragment thereof to one or a plurality of cell types disclosed herein to the binding association from or related to a subject who is known not to have or be diagnosed with immune dysfunction. In some embodiments, the step of correlating comprising comparing information about the subject with values related to protein or cell association using a database of known or predicted values related to protein or cell association. In some embodiments, any disclosed method comprises the step of characterizing, identifying, or calculating a risk that a subject will acquire or has an immune dysfunction sufficient to cause a disclosed disorder by using any of the disclosed algorithms. In some embodiments, the methods comprises a step of correlating a level of immune dysregulation with the quantity of one or a combination of: the binding of 14-3-3 eta bound to PIF or the functional fragment thereof, the binding of Myosin 9 bound to PIF or the functional fragment thereof, the binding of Thymosin-al bound to PIF or the functional fragment thereof, and the number of CD8+ cells from CD4+, CD8+, or CD14+ cells bound to PIF comprises calculating protein interactions, including direct and indirect associations, using a database of known and predicted protein interactions. In some embodiments, the methods further comprise isolating a sample from a subject prior to the step of detecting or quantifying a number of immune cells that bind to the immobilized PIF. In some embodiments, the step of isolating a sample comprises isolating one or a combination of cell populations comprising CD4+, CD8+, and CD14+ cells from blood of the subject prior to exposing the sample to immobilized PIF.

In some embodiments, the number of immune cells bound to PIF or analog thereof from a sample of a person suspected of having immune dysregulation, RPL or endometriosis is between about fifteen percent greater and about forty percent greater than the number of immune cells bound to PIF from a reference sample. In some embodiments, the number of immune cells bound to PIF or analog thereof from a sample of a person suspected of having immune dysregulation, RPL or endometriosis is between about fifteen percent greater and about forty-five percent greater than the number of immune cells bound to PIF from a reference sample. In some embodiments, the number of immune cells bound to PIF or analog thereof from a sample of a person suspected of having immune dysregulation, RPL or endometriosis is between about fifteen percent greater and about forty-five percent greater than the number of immune cells bound to PIF from a reference sample. In some embodiments, the number of immune cells bound to PIF or analog thereof from a sample of a person suspected of having immune dysregulation, RPL or endometriosis is between about fifteen percent greater and about twenty-five percent greater than the number of immune cells bound to PIF from a reference sample In some embodiments, the number of immune cells bound to PIF or analog thereof from a sample of a person suspected of having immune dysregulation, RPL or endometriosis is between about fifteen percent greater and about thirty percent greater than the number of immune cells bound to PIF from a reference sample. In some embodiments, the number of immune cells bound to PIF or analog thereof from a sample of a person suspected of having immune dysregulation, RPL or endometriosis is between about fifteen percent greater and about thirty-five percent greater than the number of immune cells bound to PIF from a reference sample. In some embodiments, the number of immune cells bound to PIF or analog thereof from a sample of a person suspected of having immune dysregulation, RPL or endometriosis is between about fifteen percent less and about forty percent less than the number of immune cells bound to PIF from a reference sample. In some embodiments, the number of immune cells bound to PIF or analog thereof from a sample of a person suspected of having immune dysregulation, RPL or endometriosis is between about fifteen percent less and about forty-five percent less than the number of immune cells bound to PIF from a reference sample. In some embodiments, the number of immune cells bound to PIF or analog thereof from a sample of a person suspected of having immune dysregulation, RPL or endometriosis is between about fifteen percent less and about twenty-five percent less than the number of immune cells bound to PIF from a reference sample. In some embodiments, the number of immune cells bound to PIF or analog thereof from a sample of a person suspected of having immune dysregulation, RPL or endometriosis is between about fifteen percent less and about thirty percent less than the number of immune cells bound to PIF from a reference sample. In some embodiments, the number of immune cells bound to PIF or analog thereof from a sample of a person suspected of having immune dysregulation, RPL or endometriosis is between about fifteen percent less and about thirty-five percent less than the number of immune cells bound to PIF from a reference sample. In some embodiments, the amount of immobilized PIF or the analog thereof is deposited at a concentration of more than about 200 micromolar, 300 micromolar, 400 micromolar, 500 micromolar, 600 micromolar, 700 micromolar, 800 micromolar, 900 micromolar, or 1000 micromolar. In some embodiments, the solid support is a dish, plate, column, or silica chip.

One embodiment of the disclosure relates to a method of identifying a female subject with a history of RPL due to immune dysregulation comprising administering an effective amount of PIF or a functional fragment thereof; and examining said PIF's binding to circulating immune cells; wherein a change of said PIF or the functional fragment thereof binding to said circulating immune cells compared to a reference indicates that said subject's history of RPL is likely due to immune dysregulation, and normal binding of said PIF or the functional fragment thereof to said circulating immune cells compared to a reference indicates that said subject's history of RPL is likely not due to immune dysregulation.

One embodiment of the disclosure relates to a method of identifying a female subject with RPL due to immune dysregulation comprising administering an effective amount of PIF or an analog thereof; and examining said PIF's binding to circulating immune cells; wherein a change of said PIF or the analog thereof binding to said circulating immune cells compared to a reference indicates that said subject's history of RPL is likely due to immune dysregulation, and normal binding of said PIF or the functional fragment thereof to said circulating immune cells compared to a reference indicates that said subject's history of RPL is likely not due to immune dysregulation.

One embodiment of the disclosure relates to a method of identifying a female subject likely to suffer from RPL due to immune dysregulation comprising administering an effective amount of PIF or a functional fragment thereof; and examining said PIF's binding to circulating immune cells; wherein a change in said PIF or the functional fragment thereof binding to said circulating immune cells compared to a reference indicates that said subject is likely to suffer from RPL due to immune dysregulation, and normal binding of said PIF or the functional fragment thereof to said circulating immune cells compared to a reference indicates that said subject is not likely to suffer from RPL due to immune dysregulation.

One embodiment of the disclosure relates to a method of identifying a subject with immune dysregulation comprising administering an effective amount of PIF or a functional fragment thereof; and examining said PIF's binding to circulating immune cells; wherein a change in said PIF or the functional fragment thereof binding to said circulating immune cells compared to a reference indicates said subject's immune dysregulation, and normal binding of said PIF or the functional fragment thereof to said circulating immune cells compared to a reference indicates a lack of said subject's immune dysregulation.

One embodiment of the disclosure relates to a method of identifying a subject with endometriosis comprising administering an effective amount of PIF or a functional fragment thereof; and examining said PIF's binding to circulating immune cells; wherein a change in said PIF or the functional fragment thereof binding to said circulating immune cells compared to a reference indicates said subject's endometriosis, and normal binding of said PIF or the analog thereof to said circulating immune cells compared to a reference indicates a lack of said subject's endometriosis.

In some embodiments, PIF binding is measured by flow cytometry after isolation of immune cells from the subject. In some embodiments, the circulating immune cells are dendritic cells. In some embodiments, the change is a decrease in PIF binding to CD 14+ and/or dendritic cells. In some embodiments, the change is an increase in PIF binding to CD4+, CD8+, and/or natural killer (NK) cells. In some embodiments, the non-detergent buffer is sulfabetaines.

### BRIEF DESCRIPTION OF THE DRAWINGS

For a complete understanding of the nature and advantages of the present disclosure, a detailed description follows in connection with the accompanying drawings:

FIGS. 1A-D illustrate that a reduction of PIF binding to dendritic cells (DCs) can represent a marker of RPL risk or correlates to elevated risk of acquiring or having RPL. In the experiment that was conducted, 4 RPL subjects showed a >10-fold increase of mDCs, while 7 RPL subjects had values similar to the HP group (0.10±0.08); no difference in the percent of pDCs was observed (0.113±0.09 in the RPL group vs. 0.116±0.03 in the HP group). Gestational age did not modify the value of either pDCs or mDCs in the HP group. PIF binding cells were reduced equally in pDCs and mDCs in the RPL group (pDC PIF+: 41.2±19.2 in the RPL group vs. 58.2±18.3 in the HP group, p=0.0381; mDC PIF+: 46.1±14.2 in the RPL group vs. 57.9±9.1 in the HP group; p=0.029). There was no relationship between the level of mDCs present in the individual RPL subject and the % of mDC PIF+.

FIG. 2 shows that binding to CD14+ cells was amplified compared to controls. No difference was observed when cells were activated. When binding to other lineages in the presence of PHA was examined as compared to the control, the binding to both CD4 and CD8 decreased, while no difference in binding to CD19 was noted.

FIGS. 3-5 illustrate an experiment wherein the effect of PIF on the percent of the subject's lymphocytes expressing a given cytokine was determined, and the results were compared to those of the healthy control. This was carried out using PIF alone and following activation by PHA. The data show a 24-96-hour experiment in a control subject, examining levels of IL10, IL4, and TNFα comparing PIF to a PIFscr control. The number of IL10+ cells significantly increased compared to the control. This increase was followed by a return to baseline 96 hours after exposure to 1 μg/mL PHA. The cytokine ratio was compared to the control; 30 nM PIF led to a decrease in the pro/anti-inflammatory ratio (TNF/IL10/IL4). In addition, when the effect of 0-4 μg/mL PHA on these cytokines was examined, a dose-dependent response was noted, wherein the maximal effect of PIF compared to control was noted at 4 μg/mL.

FIG. 6 illustrates the comparison of the RPL subject to the healthy control subject. The data showed major changes in a number of cytokines. In the presence of PHA, the TNFα/IL10 ratio decreased in both the RPL and control subjects. In contrast, in the presence of PIF, the TNFα/IL10 ratio increased in the RPL subject, but decreased in the control subject. The INFγbasal expression was higher in the RPL subject. PHA further increased the INFγbasal expression in the RPL subject, while in the control subject a fourfold increase was noted. However, in the presence of PIF, INFγbasal expression decreased almost three-fold in the RPL subject. In the RPL subject, the baseline IL4 was high; it was unaffected by PHA but reduced by PIF. In the control subject, the baseline IL4 was low; PHA increased it four-fold, while PIF reduced it by the same amount. The INFg/IL4 ratio behaved similarly

FIGS. 7A and 7B illustrate that PIF acts directly on peripheral blood mononuclear cells (PBMCs). The interaction potential between PIF and rough (Ra LPS) or smooth (055:B5 LPS) LPS was assessed via a robust and sensitive surface plasmon resonance (SPR) method. Subsequently, the two LPS molecules at 5, 25 and 100 μM concentration were passed over the PIF attached sensor. The data demonstrated no observable LPS (ligand) and PIF-sensor interaction at all concentrations tested.

FIG. 8A illustrates SPR-based analysis, which showed that PIF targets neither the receptor itself nor its downstream mediator TLR4-MD2, even when tested at high concentrations. To further confirm this lack of interaction, TLR4-MD2 surfaces were also constructed and exposed to a high concentration (0.5 mM) of PIF, as shown in FIG. 8B.

FIGS. 9A and 9B show that PIF binding to CD3 is dose-dependent. FIG. 9C shows that PIF specifically targets CD4+/CD25+/FoxP3+ cells. FIG. 9D shows the isotype control to document PIF's binding specificity to CD3.

FIG. 10A shows that FITC-PIF binding to CD3+/CD4+ cells is specific, and is not replicated by scrambled PIF (PIFscr), which served as a control. FIG. 10B documents that FITC-PIF binding to CD4+/CD25+/FoxP3+ cells is dose-dependent, and the binding is amplified in high peptide doses, as compared to scrambled PIF, which is known to have minimal binding. The use of an isotype control demonstrated the flow cytometry experiment's validity. Such data indicates that PIF specifically binds regulatory T-cells.

FIG. 11 illustrates the extraction profile of CD14+ cells. The red (upper) line is the total lysate profile, while the blue (lower) line is the filtrate, i.e. proteins that are attached to the PIF column, which are much lower in number. This decrease in number was as expected, indicating specific PIF-protein interaction.

FIGS. 12A and 12B demonstrate that in vivo cultured PIF targets the human immune system. To determine whether PIF targets the immune system in the intact mouse, FITC-PIF was injected intravenously (IV) or intra-peritoneally (IP) followed by sacrifice 5 min and 30 min later, respectively. Global distribution of PIF within the body was analyzed through imaging. Data revealed that within 5 min a major uptake of the labeled PIF was noted within the spleen and bone marrow. A major accumulation of the labeled peptide was observed in the kidney, reflecting a rapid clearance. Following IP injection, the uptake and clearance was slower than following IV administration, as expected. This indicates that the kidney is the major site of PIF clearance. FIGS. 12C and 12D further confirm that PIF directly targets the immune system in vivo. We examined FITC-PIF interaction with circulating CD45+ cells, which are regulators of T- and B-cell antigen receptor signaling in naive mice. Using two-color flow cytometry, we found that FITC-sPIF incubated with isolated circulating mouse white blood cells binds up to 25% of those cells when exposed to 12.5-50 μg/ml FITC-PIF, with no differences found among the tested peptide concentrations, 23-25%, respectively. This indicates that in naive mice, PIF targets are limited, contrary to what is observed when immunity is activated. FIG. 12D shows FITC-PIF binding.

FIG. 13 illustrates PIF binding to 14-3-3theta using bioinformatics analysis, Such data confirm that PIF binds to this class of proteins through direct interaction with the protein at a specific binding site. This binding takes place where 14-3-3 interacts with a co-ligand 2BTP.

FIGS. 14A and 14B illustrate that FITC-PIF binding to CD3+ and CD45+ cells is not affected by the pre-exposure of PBMCs to healthy serum. FIGS. 14C and 14D, in contrast, show that FITC-PIF binding is reduced following exposure to serum of patients with endometriosis. The flow cytometry data also shows a flattened pattern.

FIG. 15 illustrates the results of a cluster analysis carried out to better define the protein target groups and identify pivotal proteins which link the different groups of proteins observed. The leading interactors were vimentin, calmodulin, SET-nuclear oncogene (apoptosis inhibitor) and Myosin 9 (MYH9). This analysis identified four major groups of proteins: PDI/HSPs, vimentin/14-3-3, immune activation, and those involved in the cytoskeleton.

FIG. 16 illustrates an analysis of PIF targets identified in CD14+ cells examined to identify proteins involved in transduction of TLR4 effect. The data showed three major proteins targeted by PIF which are significant for TLR4 action: Myosin 9, Thymosin al involved in immune activation, and 14-3-3 eta.

FIGS. 17A and 17B illustrate the cluster analyses performed in association with the Table 15.

FIGS. 18A and 18B illustrate PIF's effect on NFAT expression in PBMC. The data shown therein illustrate that PIF reduces CD4-activated cells in co-activated PBMC. Data and Western blot analysis are shown.

FIG. 19 illustrates the detection of PIF in a pregnant mare (female horse) at day 12 post-insemination, as compared to that of non-pregnant mares.

FIG. 20 illustrates FITC-labeled PIF binding to mare immune cell populations in both pregnant and non-pregnant mares. The binding to monocytes is significant in both populations.

FIG. 21 illustrates a protocol of PIF administration to mice from the time of conception. PIF's effect on spontaneous pregnancy loss and LPS-induced pregnancy loss is illustrated. PIF's promotion of fetal growth in both normal and LPS-exposed pregnant mice is also illustrated.

### DETAILED DESCRIPTION

In some embodiments, the terms “preimplantation factor” and “PIF” refer to PIF-1₍₁₅₎, a 15 amino acid peptide secreted by a human embryo prior to implantation. In some embodiments, PIF is secreted only by viable embryos. It is secreted by the fetus and the placenta, and can be detected in the maternal circulation; its presence in the maternal circulation significantly correlates with live birth. PIF plays an essential role in promoting implantation by acting on the decidua, modulating local immunity, enhancing embryo-decidual adhesion, and controlling apoptosis. Beyond promoting implantation and trophoblast invasion, PIF also has autotrophic protective effects on the embryo, promoting development and negating the toxicity of serum derived from patients with a history of recurrent pregnancy loss (RPL). In addition, PIF has shown an immunomodulatory effect in a juvenile mouse model of diabetes, wherein it modulates systemic Th1/Th2 cytokines and prevents diabetes development long-term. In an autoimmune encephalitis model, PIF reverses advanced paralysis, downregulates neural proinflammatory Th1-type genes and proteins, and inhibits IL6 and IL17 secretion through direct action on activated splenocytes. A critical element for effective embryo-maternal interaction is the development of immune tolerance without immunosuppression. PIF regulates global immunity, exerting minimal effect while having a robust effect on activated systemic immunity, as demonstrated in preclinical models of autoimmune disorders, transplantation, and reversed brain injury.

In some embodiments, “preimplantation factor” or “PIF” may also refer to synthetic PIF-1, which replicates the native peptide's effect and exerts potent immune modulatory effects on activated peripheral blood mononuclear cell (PBMC) proliferation and cytokine secretion, acting through novel sites on PBMCs and having an effect which is distinct from known immunosuppressive drugs. In some embodiments, “preimplantation factor” or “PIF” refers to an amino acid selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, mimetics thereof, and combinations thereof that are about 75, 80, 81, 82, 83, 84 85, 86, 87, 88, 89, 90, 91, 92, 93, 94, 95, 96, 97, 98, or 99% homologous to any such amino acid.

During pregnancy, the maternal immune system must tolerate fetal alloantigens encoded by paternal genes. The pregnancy site is dominated by an immunosuppressive environment. Several tolerance mechanisms have been described as operating at the feto-maternal interface: the induction of apoptosis in immune cells circulating to decidua by Fas-FasL interaction, the secretion of pregnancy-specific hormones with immunomodulatory effects, the presence of complement proteins, the inhibition of natural killer (NK) cell activity by human leukocyte antigens (HLA-G and HLA-E), the inhibition of T-cell activity, and the induction of regulatory T-cell proliferation by indoleamine 2,3 dioxygenase (IDO).

Dendritic cells (DCs) are antigen-presenting cells (APCs) found in almost all peripheral tissues, as well as in primary and secondary lymphoid organs. Their function is to collect antigenic material in the periphery, and transport it to lymph nodes, where they are scanned by naive T-lymphocytes. Depending on their subset, the type of antigen, and the microenvironment, DCs can activate immunity or induce immune tolerance.

Tolerogenic DCs are involved in immune tolerance. They represent a functional state of DCs, and are defined by their ability to inhibit T-cell activation and to induce and promote regulatory T-cell development and expansion. PIF may have a role in the generation of tolerogenic DCs from peripheral blood monocytes. Naive CD14 monocytes are the primary target of PIF.

DCs at the feto-maternal interface are involved in the maintenance of immune homeostasis during pregnancy. The state of DC activation has emerged as a key player influencing the feto-maternal immunological equilibrium. Moreover, the DC function and phenotype in the mouse decidua are controlled by the effect of paracrine mediators present at the feto-maternal interface. The fetus may also induce the modulation of the phenotype and function of circulating maternal DCs. Peripheral DCs may recirculate to the thymus, contributing to the induction of acquired thymic tolerance.

Peripheral blood DCs in normal human pregnancies have been found in a state of incomplete activation characterized by the upregulation of co-stimulatory molecules and maturation markers without a concomitant upregulation of HLA-DR molecules. The inhibition of HLA-DR upregulation in monocyte-derived DCs is sustained by sera from pregnant women. It is possible that soluble circulation factors may contribute to the modulation of the state of DCs.

The percentage and ratio of peripheral blood myeloid dendritic cells (mDCs) and plasmacytoid dendritic cells (pDCs) are lower in pregnant women than in non-pregnant females. This difference may indicate that a depressed level of immunostimulatory mDCs is involved in the temporal reversal of the immunologic imbalance, or immunotolerance, between mother and fetus.

Regulatory T-cells play an important role in the immune response. They are considered important for maternal recognition of pregnancy, and are viewed as an important element in controlling immune disorders. PIF may target this important immune lineage, further supporting PIF's regulatory action. PIF's action on the immune system is thought to be direct; the CD 14, CD4, and CD8 immune lineages share the same, mostly intracellular, protein targets. PIF directly targets the immune system within a short time after its administration, and effectively interacts with systemic immunity.

The binding of a PIF peptide to a subject's circulating immune cells, whether normal, increased, or decreased, may provide information about the immune health of that subject, potentially acting as an “immune fitness sensor” for the subject.

Before the present compositions and methods are described, it is to be understood that this disclosure is not limited to the particular processes, compositions, or methodologies described, as these may vary. It is also to be understood that the terminology used in the description is for the purpose of describing the particular versions or embodiments only, and is not intended to limit the scope of the present disclosure which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference in their entirety. Nothing herein is to be construed as an admission that the disclosure is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target subject, organ, tissue or cell or to administer a therapeutic to a patient, whereby the therapeutic positively impacts the subject, organ, tissue or cell to which it is targeted. Thus, as used herein, the term “administering”, when used in conjunction with PIF, can include, but is not limited to, providing PIF into or onto the target subject, organ, tissue or cell; providing PIF systemically to a patient by, e.g., intravenous injection whereby the therapeutic reaches the target organ, tissue or cell; providing PIF in the form of the encoding sequence thereof to the target tissue (e.g., by so-called gene-therapy techniques). “Administering” may be accomplished by parenteral, oral or topical administration, or by such methods in combination with other known techniques.

The term “analog” refers to any peptidomimetic, functional fragment, mutant, variant, salt, pharmaceutical acceptable salt, polymorph, or non-naturally occurring peptide that is structurally similar to a naturally occurring full-length protein and shares at least one biochemical or biological activity of the naturally occurring full-length protein upon which the analog is based. In some embodiments, the term “analog” refers to any polypeptide comprising at least one a-amino acid and at least one non-native amino acid residue, wherein the polypeptide shares at least one biochemical or biological activity of the naturally occurring full-length protein upon which the analog is based. For instance in the case of PIF, a PIF analog may be 70% or more homologous to wild-type PIF and may share at least one binding property of wild-type PIF. PIF is known to bind to multiple receptors. Therefore, in some embodiments, the analog refers to a PIF peptidomimetic, functional fragment, mutant, variant, salt, polymorph, or non-naturally occurring peptide that is structurally similar to wild-type PIF but binds only to one of the naturally occurring ligands to which naturally occurring PIF binds.

The term “animal” or “patient” or “subject” as used herein includes, but is not limited to, humans and non-human vertebrates such as wild, domestic and farm animals In some embodiments the term “animal” or “patient” or “subject” refers to humans. In some embodiments, the subjects may be horses. In some embodiments, the subjects may be mice. In some embodiments, the subject may be a mammal which functions as a source of the isolated cell sample. In some embodiments, the subject may be a non-human animal from which a cell sample is isolated or provided. In some embodiments, the subject may be a mammal from which a cell sample is isolated or provided. The term “mammal” encompasses both humans and non-humans and includes but is not limited to humans, non-human primates, canines, felines, murines, caprine, bovines, equines, and porcines.

“Immune-modulating” or “immunomodulating” refers to the ability of a compound of the present disclosure to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including cytokines, chemokines, lymphokines and antibodies, that have autocrine, paracrine, and endocrine effects on immune cells.

An “array”, as that term is used herein, typically refers to an arrangement of entities (e.g., PIF or PIF analogs) in spatially discrete locations with respect to one another, and usually in a format that permits simultaneous exposure of the arranged entities to potential interaction partners (e.g., cells) or other reagents, substrates, etc. In some embodiments, an array comprises entities arranged in spatially discrete locations on a solid support. In some embodiments, spatially discrete locations on an array are termed “spots” (regardless of their shape). In some embodiments, spatially discrete locations on an array are arranged in a regular pattern with respect to one another (e.g., in a grid).

The term “improves” is used to convey that the present disclosure changes either the appearance, form, characteristics and/or the physical attributes of the subject, organ, tissue or cell to which it is being provided, applied or administered. For example, the change in form compared to a reference may be demonstrated by any of the following alone or in combination: a decrease in PIF binding to circulating immune cells, an increase in PIF binding to circulating immune cells, no change in PIF binding to circulating immune cells, a decrease in PIF binding to DCs, an increase in PIF binding to DCs, or no change in PIF binding to DCs.

The term “inhibiting” includes the administration of a compound of the present disclosure to prevent the onset of the symptoms, alleviating the symptoms, or eliminating the disease, condition or disorder,

As used herein, the terms “peptide,” “polypeptide” and “protein” are used interchangeably and refer to two or more amino acids covalently linked by an amide bond or non-amide equivalent. The peptides of the disclosure can be of any length. For example, the peptides can have from about two to about 100 or more residues, such as, 5 to 12, 12 to 15, 15 to 18, 18 to 25, 25 to 50, 50 to 75, 75 to 100, or more in length. Preferably, peptides are from about 2 to about 18 residues. The peptides of the disclosure include 1- and d-isomers, and combinations of 1- and d-isomers. The peptides can include modifications typically associated with post-translational processing of proteins, for example, cyclization (e.g., disulfide or amide bond), phosphorylation, glycosylation, carboxylation, ubiquitination, myristylation, or lipidation.

By “pharmaceutically acceptable,” it is meant that the carrier, diluent or excipient must be compatible with the other ingredients of the formulation or composition and not deleterious to the recipient thereof.

As used herein, the term “therapeutic” means an agent utilized to treat, combat, ameliorate, prevent or improve an unwanted condition or disease of a patient. In part, embodiments of the present disclosure are directed to methods of examining PIF binding to a subject's circulating immune cells as a marker for immune dysregulation, including immune dysregulation that may explain a subject's history of or predisposition for RPL.

A “therapeutically effective amount” or “effective amount” of a composition is a predetermined amount calculated to achieve the desired effect, i.e., to demonstrate normal or abnormal binding with the subject's circulating immune cells. The activity contemplated by the present methods includes both medical therapeutic and/or diagnostic reagent applications. The specific dose of a compound administered according to this disclosure to obtain therapeutic and/or prophylactic effects will, of course, be determined by the particular circumstances surrounding the case, including, for example, the compound administered, the route of administration, and the condition being tested. The compounds are effective over a wide dosage range and, for example, dosages administered will normally fall within the range of from 0.001 to 10 mg/kg, more usually in the range of from 0.1 to 3 mg/kg. However, it will be understood that the effective amount administered will be determined by the physician or scientist in the light of the relevant circumstances including the condition to be tested, the choice of compound to be administered, and the chosen route of administration, and therefore the above dosage ranges are not intended to limit the scope of the disclosure in any way. A therapeutically effective amount of compound of embodiments of this disclosure is typically an amount such that when it is administered in a physiologically tolerable excipient composition, it is sufficient to achieve an effective systemic concentration or local concentration in the tissue. For the purposes of diagnostic reagents, an effective amount is the amount of a compound (such as PIF or an analog thereof that, when immobilized or in solution ex vivo) is sufficient to bind to a sample or component of a sample. In some embodiments, the component of a sample may be a cell or plurality of cells.

Generally, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine: cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy or punch biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc. In some embodiments, the sample is any fluid, cell, tissue, or collection or combination thereof obtained from a subject. A sample may be obtained for the purposes of studying, diagnosing, treating, or any other purpose. Any of the disclosed methods herein may comprise a step of obtaining or isolating a sample prior to the step of exposing the sample to one or a plurality of PIF peptides or analogs thereof. Samples include, but are not limited to, those of blood, blood components, plasma, cells, tissue, hair, skin, urine, or feces. Samples may be obtained by methods such as venipuncture, biopsy, fluid collection, buccal swab, finger-stick, or any other means. In some embodiments, the sample is from a pregnant female and compared to a similar or same type of sample taken from a non-pregnant female. In some embodiments, the sample is taken from a human or other mammal (such as a cow or horse). In some embodiments, the sample comprises a one or a plurality of placental cells, endometrial cells, splenic cells, blood cells, lymph cells or immune cells.

The terms “treat,” “treated,” or “treating” as used herein refer to both therapeutic treatment and prophylactic or preventative measures, wherein the object is to prevent or slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As used herein, the terms “sufficient” and “sufficient to cause” generally describe a phenomenon, condition, treatment, or intervention adequate to effect a known outcome. The term “sufficient to cause endometriosis” means the level of immune dysfunction that correlates with endometriosis. The term “sufficient to cause RPL” means the level of immune dysfunction that correlates with recurrent pregnancy loss (RPL). The term “sufficient to cause immune dysfunction” means the level of decreased immunity sufficient to cause immune dysfunction.

As used herein, the term “binding event” refers to association of, covalently or non-covalently, between or among at least two different molecules. In some embodiment, binding refers to passive electrostatic non-covalent binding. In some embodiments, a binding event is a measure of the tightness with which a particular ligand binds to (e.g., associates non-covalently with) and/or the rate or frequency with which it dissociates from, one or more partners. As is known in the art, any of a variety of technologies can be utilized to determine a binding event. In many embodiments, a binding event represents a measure of affinity. In some embodiments a binding event is an affinity measured between a cell and PIF or an analog thereof. In some embodiments, a binding event of cells to PIF or an analog thereof is expressed relative to binding affinities of cells to other peptides. In some embodiments, a relative binding event of cells to PIF or an analog thereof is expressed as a fold change relative to an average of all binding events of cells to peptides assayed. In some embodiments, a binding event is a relative binding affinity. In some embodiments, the binding affinity is 0. In some embodiments, a relative binding affinity is from about 0 to about 1. In some embodiments, a relative binding affinity is about 1, 2, 3, 4, 5, 6, 7, 8, 9, 10 or more fold difference as compared to a control or series of controls. In some embodiments, a relative binding affinity is from about 0 and to about −1. In some embodiments, a relative binding affinity is about −1, −2, −3, −4, −5, −6, −7, −8, −9, −10 or more fold difference as compared to a control or series of controls.

As used herein, the term “binding profile” refers to a collection of data representing one or a plurality of values that correlate to the association of two or more molecules. In some embodiments, a binding profile is associated with a sample from a subject. In some embodiments, the term “binding profile” refers to a set of data comprising one or a plurality of characteristic ways in which an amino acid sequence (such as PIF or a functional fragment thereof) binds, adheres, adsorbs, or interacts to a biomolecule and/or cell, including an immune cell or a protein expressed by an immune cell.

Generally speaking, the term “tissue” refers to any aggregation of similarly specialized cells which are united in the performance of a particular function,

As used herein, “immune cells” are those cells which are involved in an immune response. In some embodiments, the immune cells comprises one or a combination of cell populations selected from: peripheral blood mononuclear cells (PBMCs), granulocytes, basophils, eosinophils, neutrophils, mast cells, monocytes, macrophages, antigen-presenting cells (APCs), dendritic cells (DCs), B-cells, T-cells, natural killer (NK) cells, cells that express one or plurality of TLRs, TCRs, or BCRs. The immune response may be adaptive or innate, and the involved cells may include, but are not limited to, granulocytes, basophils, eosinophils, neutrophils, mast cells, monocytes, macrophages, antigen-presenting cells (APCs), dendritic cells (DCs), B-cells, T-cells, natural killer (NK) cells, antibodies, lymphocytes, cytokines, toll-like receptors (TLRs), B-cell receptors (BCRs), T-cell receptors (TCRs), regulatory T-cells, and any other cells that may be involved in an immune response.

As used herein, “solid support” refers to the stationary phase of a separation method, and is a non-aqueous matrix onto which an amino acid sequence is capable of being immobilized. Such supports include agarose, sepharose, glass, silica, polystyrene, collodion charcoal, bead, sand, and any other suitable material. Any suitable method can be used to affix or to absorb the amino acid sequence to the solid support and retain at least a portion of its ability to bind to a ligand or molecule. A solid support may be in the form of a dish, plate, column, silica chip, or any other suitable form optionally comprising any matrix material that is sufficient to cross link peptides to the surface.

As used herein, the term “functional fragment” means any portion of an amino acid sequence that is of a sufficient length to retain at least partial biological function that is similar to or substantially similar to the wild-type polypeptide upon which the fragment is based. In some embodiments, a functional fragment of a polypeptide associated with the extracellular matrix is a polypeptide that comprises 75, 80, 85, 90, 91, 92, 93, 94, 95, 96, 97, 98, or 99% sequence identity of any polypeptide disclosed in Table 4 and has sufficient length to retain at least partial binding to one or a plurality of ligands that bind to the polypeptide in Table 4. In some embodiments, the fragment is a fragment of any polypeptide disclosed in Table 4 and has a length of no more than about 20, about 19, about 18, about 17, about 16, about 15, about 14, about 13, about 12, about 11, about 10, about 9, about 8, about 7, about 6, about 5, about 4, about 3, or about 2 contiguous amino acids. In some embodiments, the fragment is a fragment of any polypeptide disclosed in Table 4 and has a length of no more than about 20 amino acids. In some embodiments, the fragment is a fragment of any polypeptide disclosed in Table 4 and has a length of no more than about 19 amino acids. In some embodiments, the fragment is a fragment of any polypeptide disclosed in Table 4 and has a length of no more than about 18 amino acids. In some embodiments, the fragment is a fragment of any polypeptide disclosed in Table 4 and has a length of no more than about 17 amino acids. In some embodiments, the fragment is a fragment of any polypeptide disclosed in Table 4 and has a length of no more than about 16 amino acids. In some embodiments, the fragment is a fragment of any polypeptide disclosed in Table 4 and has a length of no more than about 15 amino acids. In some embodiments, the fragment is a fragment of any polypeptide disclosed in Table 4 and has a length of no more than about 14 amino acids. In some embodiments, the fragment is a fragment of any polypeptide disclosed in Table 4 and has a length of no more than about 13 amino acids. In some embodiments, the fragment is a fragment of any polypeptide disclosed in Table 4 and has a length of no more than about 12 amino acids. In some embodiments, the fragment is a fragment of any polypeptide disclosed in Table 4 and has a length of no more than about 11 amino acids. In some embodiments, the fragment is a fragment of any polypeptide disclosed in Table 4 and has a length of no more than about 10 amino acids. In some embodiments, the fragment is a fragment of any polypeptide disclosed in Table 4 and has a length of no more than about 9 amino acids. In some embodiments, the fragment is a fragment of any polypeptide disclosed in Table 4 and has a length of no more than about 8 amino acids. In some embodiments, the fragment is a fragment of any polypeptide disclosed in Table 4 and has a length of no more than about 7 amino acids. In some embodiments, the fragment is a fragment of any polypeptide disclosed in Table 4 and has a length of no more than about 6 amino acids. In some embodiments, the fragment is a fragment of any polypeptide disclosed in Table 4 and has a length of no more than about 5 amino acids. In some embodiments, the fragment is a fragment of any polypeptide disclosed in Table 4 and has a length of no more than about 4 amino acids. In some embodiments, the fragment is a fragment of any polypeptide disclosed in Table 4 and has a length of no more than about 3 amino acids. In some embodiments, the fragment is a fragment of any polypeptide disclosed in Table 4 and has a length of no more than about 2 amino acids.

As used herein, “examining” means the act of observing, quantifying and/or detecting the presence or absence of a particular physical feature of, between or among one or a plurality of elements. In the case of the disclosed methods, in some embodiments, the act of examining refers to monitoring, observing, and/or measuring the degree to which PIF or a functional fragment thereof binds, associates or otherwise interacts with a molecule, amino acid sequence, and/or cell.

As used herein, “classifying” means the act of assigning or characterizing or associating a group of people, subjects, and/or entities with a certain condition(s), characteristic(s), and/or physical feature.

As used herein, “exposing” means the act of laying an element open to something. In some embodiments, exposing refers to placing the element in an environment and under conditions sufficient to enable contact between the element and another substance, reagent, condition, or stimulus. In some embodiments, the term exposing comprises contacting PIF or a functional fragment thereof to a substance, reagent, or condition such that the contact produces an effect. In some embodiments, exposing comprises administering an effective amount of PIF to a subject.

As used herein, “comparing” means the act of estimating, measuring, or assessing the similarity or dissimilarity between two elements. In some embodiments of the disclosure, the step of comparing comprises collecting and/or analyzing and/or normalizing data against control data as applied in an experiment, group of experiments, or algorithm used in such experiments, such that quantities are measured and/or values corresponding to those quantities are assigned to a feature, condition, mode, control or variable of the experiment(s). In some embodiments, comparing comprises observing the similarity or dissimilarity between or among two or more data points and/or values.

As used herein, “immune dysregulation” means a disease or disorder or condition characterized by an immunological imbalance in a subject. In some embodiments, immune dysregulation refers to an immunological imbalance in a subject caused by an acquired, environmental factor (such as a pathogen) and/or a genetic factor. In some embodiments, immune dysregulation refers to abnormal immune cell function as compared to a control. In some embodiments, the abnormal immune cell function may manifest by an improper clonal expansion of T cells capable of generating an antigen-specific immune response. In some embodiments, immune dysregulation comprises an improper innate immune system reaction capable of making the subject more susceptible to acquiring or experiencing a condition, such as recurrent pregnancy loss. Diseases that may be caused by immune dysregulation may include, for example, Hashimoto's thyroiditis, pernicious anemia, Addison's disease, type I (insulin-dependent) diabetes, rheumatoid arthritis, systemic lupus erythematosus, dermatomyositis, Sjogren's syndrome, lupus erythematosus, multiple sclerosis, myasthenia gravis, Reitcer's syndrome, Grave's disease, alopecia greata, anklosing spondylitis, antiphospholipid syndrome, auto-immune hemolytic anemia, auto-immune hepatitis, autoimmune inner ear disease, autoimmune lymphoproliferative-syndrome (ALPS), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyncuropathy, cicatrical pemphigoid, cold agglutinin disease, CREST syndrome, Crohn's disease, Dego's disease, discoid lupus, essential mixed cryoglobulinemia, fibromyalgia-fibromyositis, Guillain-Barre syndrome, idiopathic pulmonary fibrosis, idiopathic thrombocytopenia purpura (ITP), IgA nephropathy, juvenile arthritis, Meniere's disease, mixed connective tissue disease, pemphigus vulgaris, polyarteritis nodosa, polychondritis, polyglancular syndromes, polymyalgia rheumatica, polymyositis, primary agammaglobulinemia, primary biliary cirrhosis, psoriasis, Raynaud's phenomenon, rheumatic fever, sarcoidosis, scleroderma, stiff-man syndrome, Takayasu arteritis, temporal arteritis/giant cell arteritis, ulcerative colitis, uveitis, vasculitis, vitiligo, and Wegener's granulomatosis.

This application describes compounds. Without being bound by any particular theory, the compounds described herein act as agonists of PIF-mediated signal transduction via the receptor or receptors of PIF. Thus, these compounds modulate signaling pathways that provide significant therapeutic benefit in the treatment of, but not limited to, RPL, endometriosis, and immune dysregulation. The compounds of the present disclosure may exist in unsolvated forms as well as solvated forms, including hydrated forms. The compounds of the present disclosure also are capable of forming both pharmaceutically acceptable salts, including but not limited to acid addition and/or base addition salts. Furthermore, compounds of the present disclosure may exist in various solid states including an amorphous form (non-crystalline form), and in the form of clathrates, prodrugs, polymorphs, bio-hydrolyzable esters, racemic mixtures, non-racemic mixtures, or as purified stereoisomers including, but not limited to, optically pure enantiomers and diastereomers. In general, all of these forms can be used as an alternative form to the free base or free acid forms of the compounds, as described above and are intended to be encompassed within the scope of the present disclosure.

A “polymorph” refers to solid crystalline forms of a compound. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Different physical properties of polymorphs can affect their processing. In some embodiments, the pharmaceutical composition comprises at least one polymorph of any of the compositions disclosed herein.

As noted above, the compounds of the present disclosure can be administered, inter alia, as pharmaceutically acceptable salts, esters, amides or prodrugs. The term “salts” refers to inorganic and organic salts of compounds of the present disclosure. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitiate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977). The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Salts include those formed from hydrochloric, hydrobromic, sulphuric, phosphoric, citric, tartaric, lactic, pyruvic, acetic, succinic, fumaric, maleic, methanesulphonic and benzenesulphonic acids.

In some embodiments, salts of the compositions comprising either a PIF or PIF analog or PIF mutant may be formed by reacting the free base, or a salt, enantiomer or racemate thereof, with one or more equivalents of the appropriate acid. In some embodiments, pharmaceutical acceptable salts of the present disclosure refer to analogs having at least one basic group or at least one basic radical. In some embodiments, pharmaceutical acceptable salts of the present disclosure comprise a free amino group, a free guanidino group, a pyrazinyl radical, or a pyridyl radical that forms acid addition salts. In some embodiments, the pharmaceutical acceptable salts of the present disclosure refer to analogs that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, malcic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane-or 2-hydroxyethane-sulfonic acid, or aromatic sulfonic acids, for example benzene-, p-toluene- or naphthalene-2-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present disclosure may be found in their anhydrous form or as in hydrated crystalline form (i.e., complexed or crystallized with one or more molecules of water). In some embodiments, salts of PIF may be immobilized to a solid support or in solution resuspended in a pharmaceutically acceptable carrier and used in any method disclosed herein.

Examples of pharmaceutically acceptable esters of the compounds of the present disclosure include C₁-C₈ alkyl esters. Acceptable esters also include C₅-C₇ cycloalkyl esters, as well as arylalkyl esters such as benzyl. C₁-C₄ alkyl esters are commonly used. Esters of compounds of the present disclosure may be prepared according to methods that are well known in the art. Examples of pharmaceutically acceptable amides of the compounds of the present disclosure include amides derived from ammonia, primary C₁-C₈ alkyl amines, and secondary C₁-C₈ dialkyl amines. In the case of secondary amines, the amine may also be in the form of a 5 or 6 membered heterocycloalkyl group containing at least one nitrogen atom. Amides derived from ammonia, C₁-C₃ primary alkyl amines and C₁-C₂ dialkyl secondary amines are commonly used. Amides of the compounds of the present disclosure may be prepared according to methods well known to those skilled in the art.

As used herein, “conservative” amino acid substitutions may be defined as set out in Tables 1-3 below. The PIF compounds of the disclosure include those wherein conservative substitutions (from either nucleic acid or amino acid sequences) have been introduced by modification of polynucleotides encoding polypeptides of the disclosure. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has similar binding affinities. Exemplary conservative substitutions are set out in Table 1.

TABLE 1
Conservative Substitutions 1

| Side Chain Characteristics | Amino Acid |
|---|---|
| Aliphatic | Amino Acid |
| Non-polar | GAPILVF |
| Polar-uncharged | CSTMNQ |
| Polar-charged | DEKR |
| Aromatic | HFWY |
| Other | NQDE |

Alternately, conservative amino acids can be grouped as described in Lehninger, (Biochemistry, Second Edition; Worth Publishers, Inc. NY, N.Y. (1975), pp. 71-77) as set forth in

TABLE 2
Conservative Substitutions II

| Side Chain Characteristic | Amino Acid |
|---|---|
| Non-polar (hydrophobic) | |
| Aliphatic: | ALIVP |
| Aromatic: | FWY |
| Sulfur-containing: | M |
| Borderline: | GY |
| Uncharged-polar | |
| Hydroxyl: | STY |
| Amides: | NQ |
| Sulfhydryl: | C |
| Borderline: | GY |
| Positively Charged (Basic): | KRH |
| Negatively Charged (Acidic): | DE |

Alternately, exemplary conservative substitutions are set out in Table 3.

TABLE 3
Conservative Substitutions III

| Original Residue | Exemplary Substitution |
|---|---|
| Ala (A) | Val Leu He Met |
| Arg(R) | Lys His |
| Asn (N) | Gin |
| Asp (D) | Glu |
| Cys (C) | Ser Thr |
| Gin (Q) | Asn |
| Glu (E) | Asp |
| Gly(G) | Ala Val Leu Pro |
| His (H) | Lys Arg |
| He (I) | Leu Val Met Ala Phe |
| Leu (L) | He Val Met Ala Phe |
| Lys (K) | Arg His |
| Met (M) | Leu He Val Ala |
| Phe (F) | Trp Tyr He |
| Pro (P) | Gly Ala Val Leu He |

As used herein, the terms “peptide,” “polypeptide” and “protein” are used interchangeably and refer to two or more amino acids covalently linked by an amide bond or non-amide equivalent. The peptides of the disclosure can be of any length. For example, the peptides can have from about two to about 100 or more residues, such as, 5 to 12, 12 to 15, 15 to 18, 18 to 25,25 to 50,50 to 75,75 to 100, or more in length. Preferably, peptides are from about 2 to about 18 residues in length. The peptides of the disclosure also include 1- and d-isomers, and combinations of 1- and d-isomers. The peptides can include modifications typically associated with posttranslational processing of proteins, for example, cyclization (e.g., disulfide or amide bond), phosphorylation, glycosylation, carboxylation, ubiquitination, myristylation, or lipidation. In some embodiments, the compositions or pharmaceutical compositions of the disclosure relate to analogs of any PIF sequence set forth in Table 4 that share no less than about 70%, about 75%, about 79%, about 80%, about 85%, about 86%, about 87%, about 90%, about 93%, about 94% about 95%, about 96%, about 97%, about 98%, about 99% homology with any one or combination of PIF sequences set forth in Table 4. In some embodiments, PIF or PIF peptide may refer to an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, mimetics thereof, or a functional fragment thereof that is about 70%, 75%, 80%, 85%, 90%, 91%, 92%, 93%, 94%, 95%, 96%, 97%, 98%, or 99% homologous to any such amino acid sequence. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 20. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 21. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 22. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 23. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 24. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98% or 99% homologous to SEQ ID. No: 25. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 26. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 27. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 28. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 29. In some embodiments, the PIF mutant comprises a sequence selected from: XVZIKPGSANKPSD, XVZIKPGSANKPS XVZIKPGSANKP XVZIKPGSANK XVZIKPGSAN, XVZIKPGSA, XVZIKPGS, XVZIKPG, XVZIKP, XVZIK, XVZI, XVZ wherein X is a non-natural amino acid or a naturally occurring amino acid. In some embodiments, the PIF mutant comprises a sequence selected from: XVZIKPGSANKPSD, XVZIKPGSANKPS XVZIKPGSANKP XVZIKPGSANK XVZIKPGSAN, XVZIKPGSA, XVZIKPGS, XVZIKPG, XVZIKP, XVZIK, XVZI, XVZ wherein X is a non-natural amino acid or a naturally occurring amino acid except that X is not methionine if Z is arginine, and Z is not arginine if X is methionine. In some embodiments, the PIF analog or mutant is synthetic or synthetically made.

Peptides disclosed herein further include compounds having amino acid structural and functional analogs, for example, peptidomimetics having synthetic or non-natural amino acids (such as a norleucine) or amino acid analogues or non-natural side chains, so long as the mimetic shares one or more functions or activities of compounds of the disclosure. The compounds of the disclosure therefore include “mimetic” and “peptidomimetic” forms. As used herein, a “non-natural side chain” is a modified or synthetic chain of atoms joined by covalent bond to the a-carbon atom, (3-carbon atom, or y-carbon atom which does not make up the backbone of the polypeptide chain of amino acids. The peptide analogs may comprise one or a combination of non-natural amino-acids chosen from: norvaline, tert-butylglycine, phenylglycine, He, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, N-methyl-lysine, homocysteine, and Tyr; Xaa2 is absent, or an amino acid selected from the group consisting of Ala, D-Ala, N-methyl-alanine, Glu, N-methyl-glutamate, D-Glu, Gly, sarcosine, norleucine, Lys, D-Lys, Asn, D-Asn, D-Glu, Arg, D-Arg, Phe, D-Phe, N-methyl-phenylalanine, Gin, D-Gln, Asp, D-Asp, Ser, D-Ser, N-methyl-serine, Thr, D-Thr, N-methyl-threonine, D-Pro D-Leu, N-methyl-leucine, D-Ile, N-methyl-isoleucine, D-Val, N-methyl-valine, tert-butylglycine, D-tert-butylglycine, N-methyl-tert-butylglycine, Trp, D-Trp, N-methyl-tryptophan, D-Tyr, N-methyl-tyrosine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (5)-2-amino-3-(1H-tetrazol-5-yl)propanoic acid, Glu, Gly, N-methyl-glutamate, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, octylglycine, tranexamic acid, aminovaleric acid, and 2-(2-aminoethoxy)acetic acid. The natural side chain, or R group, of an alanine is a methyl group. In some embodiments, the non-natural side chain of the composition is a methyl group in which one or more of the hydrogen atoms is replaced by a deuterium atom. Non-natural side chains are disclosed in the art in the following publications: WO/2013/172954, WO2013123267, WO/2014/071241, WO/2014/138429, WO/2013/050615, WO/2013/050616, WO/2012/166559, US Application No. 20150094457, Ma. Z., and Hartman. M. C. (2012). In Vitro Selection of Unnatural Cyclic Peptide Libraries via mRNA Display. In J. A. Douthwaite & R. I I. Jackson (Eds.). Ribosome Display and Related Technologies: Methods and Protocols (pp. 367-390). Springer N.Y., all of which are incorporated by reference in their entireties.

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics, as further described below. In some embodiments, the compositions, pharmaceutical compositions and kits comprise a peptide or peptidomimeic or analog shareing share no less than about 70%, about 75%, about 79%, about 80%, about 85%, about 86%, about 87%, about 90%, about 93%, about 94% about 95%, about 96%, about 97%, about 98%, about 99% homology with any one or combination of PIF sequences set forth in Table 4; and wherein one or a plurality of amino acid residues is a non-natural amino acid residue or an amino acid residue with a non-natural sidechain. In some embodiments, peptide or peptide mimetics are provided, wherein a loop is formed between two cysteine residues. In some embodiments, the peptidomimetic may have many similarities to natural peptides, such as: amino acid side chains that are not found among the known 20 proteinogenic amino acids, non-peptide-based linkers used to effect cyclization between the ends or internal portions of the molecule, substitutions of the amide bond hydrogen moiety by methyl groups (N-methylation) or other alkyl groups, replacement of a peptide bond with a chemical group or bond that is resistant to chemical or enzymatic treatments, N- and C-terminal modifications, and conjugation with a non-peptidic extension (such as polyethylene glycol, lipids, carbohydrates, nucleosides, nucleotides, nucleoside bases, various small molecules, or phosphate or sulfate groups). As used herein, the term “cyclic peptide mimetic” or “cyclic polypeptide mimetic” refers to a peptide mimetic that has as part of its structure one or more cyclic features such as a loop, bridging moiety, and/or an internal linkage. As used herein, the term “bridging moiety” refers to a chemical moiety that chemically links one or a combination of atoms on an amino acid to any other atoms outside of the amino acid residue. For instance, in the case of an amino acid tertiary structure, a bridging moiety may be a chemical moiety that chemically links one amino acid side chain with another sequential or non-sequential amino acid side chain.

In some embodiments, peptide or peptide mimetics are provided, wherein the loop comprises a bridging moiety selected from the group consisting of:

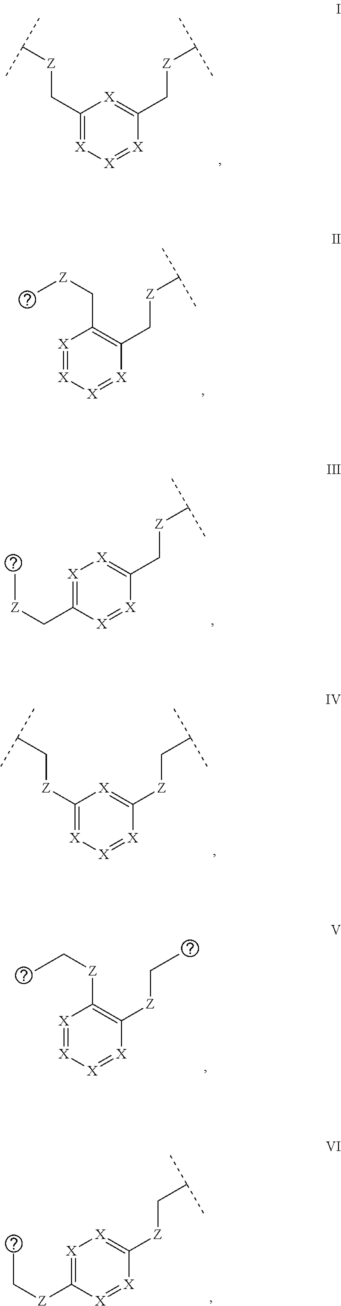

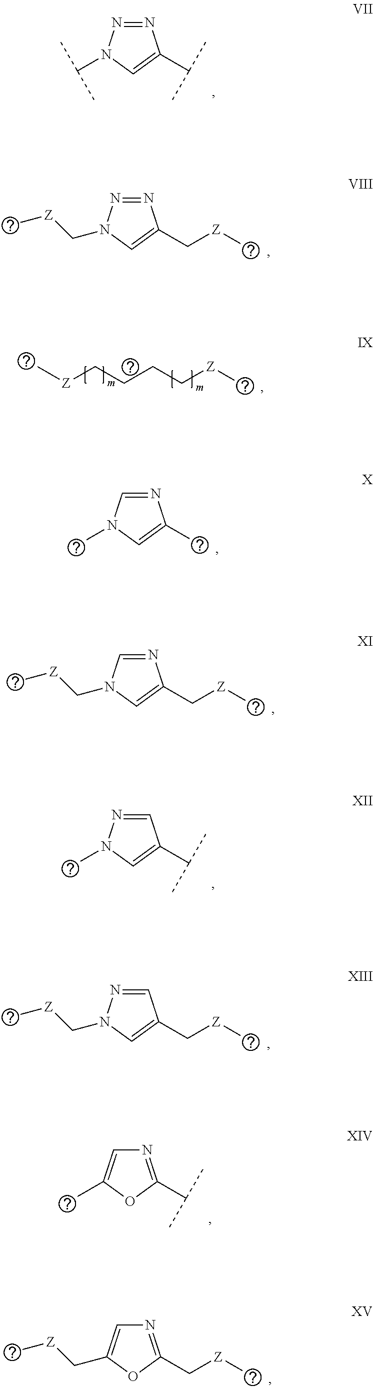

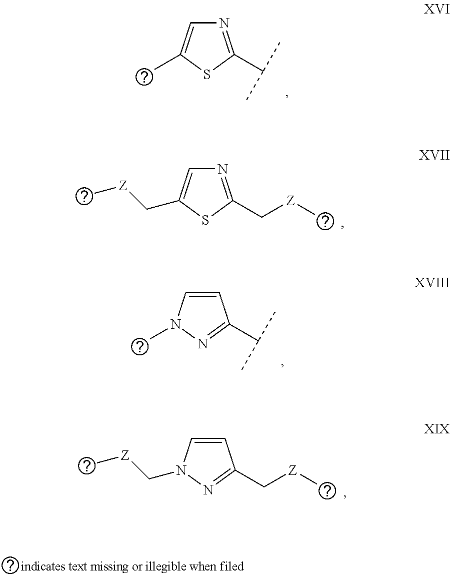

wherein each X is independently N or CH, such that no ring contains more than 2 N; each Z is independently a bond, NR, 0, S, CH2, C(0)NR, NRC(0), S(0)vNR, NRS(0)v; each m is independently selected from 0, 1, 2, and 3; each vis independently selected from 1 and 2; each R is independently selected from Hand C₁-C₆; and each bridging moiety is connected to the peptide by independently selected C₀-C₆ spacers.

In some embodiments, the PIF peptides of the disclosure are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7 membered alkyl, amide, amide lower alkyl, amide di (lower alkyl), lower alkoxy, hydroxy, carboxy and the lower ester derivatives thereof, and with 4-, 5-, 6-, to 7 membered heterocyclics. For example, proline analogs can be made in which the ring size of the proline residue is changed from 5 members to 4, 6, or 7 members. Cyclic groups can be saturated or unsaturated, and if unsaturated, can be aromatic or nonaromatic. Heterocyclic groups can contain one or more nitrogen, oxygen, and/or sulphur heteroatoms. Examples of such groups include the furazanyl,furyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

In a further embodiment a compound of the formula R₁-R₂-R₃-R₄-R₅-R.₆-R₇-R₈-R₉-R₁₀-R₁₁-R₁₂-R₁₃-R₁₄-R₁₅, wherein R₁ is Met or a mimetic of Met, R₂ is Val or a mimetic of Val, R₃ is Arg or a mimetic of Arg, or any amino acid, R₄ is He or a mimetic of He, R₃ is Lys or a mimetic of Lys, R₆ is Pro or a mimetic of Pro, R₇ is Gly or a mimetic of Gly, R₈ is Ser or a mimetic of Ser, R₉ is Ala or a mimetic of Ala, R₁₀ is Asn or a mimetic of Asn, R₁₁ is Lys or a mimetic of Lys, R₁₂ is Pro or a mimetic of Pro, R₁₃ is Ser or a mimetic of Ser, R₁₄ is Asp or a mimetic of Asp and R₁₅ is Asp or a mimetic of Asp is provided. In a further embodiment, a compound comprising the formula R₁-R₂-R₃-R₄-R₅-R₆-R₇-R₈-R₉-R₁₀, wherein R₁ is Ser or a mimetic of Ser, R₂ is Gin or a mimetic of Gin, R₃ is Ala or a mimetic of Ala, R4 is Val or a mimetic of Val, R₅ is Gin or a mimetic of Gin, R₆ is Glu or a mimetic of Glu, R₇ is His or a mimetic of His, R₈ is Ala or a mimetic of Ala, R₉ is Ser or a mimetic of Ser, and R₁₀ is Thr or a mimetic of Thr; a compound comprising the formula R₁-R₂-R₃-R₄-R₅-R₆-R₇-R₈-R₉-R₁₀-R₁₁-R₁₂-R₁3-R₁₄-R₁₅-R₁₆-R₁₇-R₁₈, wherein R₁ is Ser or a mimetic of Ser, R₂ is Gly or a mimetic of Gly, R₃ is lie or a mimetic of He, R₄ is Val or a mimetic of Val, R₅ is He or a mimetic of He, R₆ is Tyr or a mimetic of Tyr, R₇ is Gin or a mimetic of Gin, R₈ is Tyr or a mimetic of Tyr, R₉ is Met or a mimetic of Met, R₁₀ is Asp or a mimetic of Asp, R₁₁ is Asp or a mimetic of Asp, R₁₂ is Arg or a mimetic of Arg, R₁₃ is Tyr or a mimetic of Tyr, R₁₄ is Val or a mimetic of Val, R₁₅ is Gly or a mimetic of Gly, R₁₆ is Ser or a mimetic of Ser, R₁₇ is Asp or a mimetic of Asp and R₁₈ is Leu or a mimetic of Leu; and a compound comprising the formula R1-R₂-R₃-R₄-R₅-R₃-R₇-R₈-R₉, wherein R₁ is Val or a mimetic of Val, R₂ is He or a mimetic of He, R₃ is He or a mimetic of He, R₄ is lie or a mimetic of He, R₅ is Ala or a mimetic of Ala, R₆ is Gin or a mimetic of Gin, R₇ is Tyr or a mimetic of Tyr, R₈ is Met or a mimetic of Met, and R₉ is Asp or a mimetic of Asp is provided. In some embodiments, R₃ is not Arg or a mimetic of Arg.

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chern. 24,243-252,1989). Certain peptidomimetic compounds are based upon the amino acid sequence of the peptides of the disclosure. Often, peptidomimetic compounds are synthetic compounds having a three dimensional structure (i.e. a “peptide motif) based upon the three-dimensional structure of a selected peptide. The peptide motif provides the peptidomimetic compound with the desired biological activity, i.e., binding to PIF receptors, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life.

Peptidomimetic design strategies are readily available in the art (see, e.g., Ripka & Rich, Curr. Op. Chern. Bioi. 2,441-452,1998; Hruby et al., Curr. Op.Chem. Bioi. 1,114-119,1997; Hruby & Baise, Curr.Med. Chern. 9,945-970,2000). One class of peptidomimetics is a backbone that is partially or completely non-peptide, but mimics the peptide backbone atom-for atom and comprises side groups that likewise mimic the functionality of the side groups of the native amino acid residues. Several types of chemical bonds, e.g., ester, thioester, thioamide, retroamide, reduced carbonyl, dimethylene and ketomethylene bonds, are known in the art to be generally useful substitutes for peptide bonds in the construction of pro tease-resistant peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as “topographical” mimetics of the original peptide (Ripka & Rich, 1998, supra).

The first natural PIF compound identified, termed nPIF (SEQ ID NO: 1), is a 15 amino acid peptide. A synthetic version of this peptide, sPIF (SEQ ID NO:13), showed activity that was similar to the native peptide, nPIF (SEQ ID NO: I). This peptide is homologous to a small region of the Circumsporozoite protein, a malaria parasite. The second PIF peptide (SEQ ID NO:7), includes 13 amino acids and shares homology with a short portion of a large protein named thyroid and retinoic acid transcription co-repressor, which is identified as a receptor-interacting factor, (SMRT); the synthetic version is sPIF-2 (SEQ ID NO: 14). The third distinct peptide, nPIF-3 (SEQ ID NO: 10), consists of 18 amino acids and matches a small portion of reverse transcriptase; the synthetic version of this peptide sPIF-3 is (SEQ ID NO: 15). nPIF-4 (SEQ ID NO: 12) shares homology with a small portion of reverse transcriptase.

A list of PIF peptides, both natural and synthetic, are provided below in Table 4. Antibodies to various PIF peptides and scrambled PIF peptides are also provided.

TABLE 4
PIF Peptides

| (SEQ ID NO) | Peptide | Amino Acid Sequence |
|---|---|---|
| SEQ ID NO: 1 | nPIF-1₁₅ | MVRIKPGSANKPSDD |
| isolated native, matches region of | | |
| Circumsporozoite protein (Malaria) | | |
| SEQ ID NO: 2 | nPIF-1₍₁₅₋ₐₗₜₑᵣ₎ | MVRIKYGSYNNKPSD |
| isolated native, matches region of | | |
| Circumsporozoite protein (Malaria) | | |
| SEQ ID NO: 3 | nPIF-1₍₁₃₎ | MVRIKPGSANKPS |
| isolated native, matches region of | | |
| Circumsporozoite protein (Malaria) | | |
| SEQ ID NO: 4 | nPIF-1₍₉₎ | MVRIKPGSA |
| isolated native, matches region of | | |
| Circumsporozoite protein (Malaria) | | |
| SEQ ID NO: 5 | scrPIF-1₍₁₅₎ | GRVDPSNKSMPKDIA |
| synthetic, scrambled amino acid sequence | | |
| from region of Circumsporozoite protein | | |
| Malaria | | |
| SEQ ID NO: 6 | nPIF-2₍₁₀₎ | SQAVQEHAST |
| isolated native, matches region of human | | |
| retinoid and thyroid hormone receptor- | | |
| SMRT | | |
| SEQ ID NO: 7 | nPIF-2₍₁₃₎ | SQAVQEHASTNMG |
| isolated native, matches region of human | | |
| retinoid and thyroid hormone receptor | | |
| (SMRT) | | |
| SEQ ID NO: 8 | nPIF-2₍₁₃₎ | EVAQHSQASTMNG |
| synthetic, scrambled amino acid sequence | | |
| from region of human retinoid and thyroid | | |

This disclosure relates, among other things, to PIF or an analog thereof used as diagnostic reagent in solid phase or liquid solution to detect the number immune cells in a sample or to stimulate cytokine expression from immune cells in a sample. In another embodiment, a pharmaceutical composition comprising a PIF peptide or analog is provided. In some embodiments, the pharmaceutical composition comprises a therapeutically effective amount of a PIF peptide or a pharmaceutically acceptable salt thereof. In some embodiments, the pharmaceutical compositions is free of a peptide comprising any one or more of the sequence identifiers of Table 4. In some embodiments, the pharmaceutical compositions is free of a peptide comprising or consisting of SEQ ID NO: 1.

For therapeutic treatment of the specified indications, an active agent may be administered as such, or can be compounded and formulated into pharmaceutical compositions in unit dosage form for parenteral, transdermal, rectal, nasal, local intravenous administration, or, preferably, oral administration. Such pharmaceutical compositions are prepared in a manner well known in the art and comprise at least one or a combination of active agents from Table 5 associated with a pharmaceutically carrier. The term “active compound”, as used throughout this specification, refers to at least one compound selected from compounds of the formulas or pharmaceutically acceptable salts thereof.

In such a composition, the active compound is known as the “active ingredient.” In making the compositions, the active ingredient will usually be mixed with a carrier, or diluted by a carrier, or enclosed within a carrier that may be in the form of a capsule, sachet, paper or other container. When the carrier serves as a diluent, it may be a solid, semisolid, or liquid material that acts as a vehicle, excipient of medium for the active ingredient. Thus, the composition can be in the form of tablets, pills, powders, lozenges, sachets, cachets, elixirs, emulsion, solutions, syrups, suspensions, soft and hard gelatin capsules, sterile injectable solutions, and sterile packaged powders.

The terms “pharmaceutical preparation” and “pharmaceutical composition” include preparations suitable for administration to mammals, e.g., humans. When the compounds of the present disclosure are administered as pharmaceuticals to mammals, e.g., humans, they can be given per se or as a pharmaceutical composition containing, for example, from about 0.1 to about 99.5% of active ingredient in combination with a pharmaceutically acceptable carrier.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. In some embodiments, the pharmaceutical compositions comprising a PIF peptide, mimetic or pharmaceutically acceptable salt thereof and at least one pharmaceutically acceptable carrier.

The phrase “pharmaceutically acceptable carrier” is art-recognized and includes a pharmaceutically acceptable material, composition or vehicle, suitable for administering compounds of the present disclosure to mammals The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other nontoxic compatible substances employed in pharmaceutical formulations. Suitable pharmaceutical carriers are described in “Remington's Pharmaceutical Sciences” by E. W. Martin, which is incorporated herein by reference in its entirety. In some embodiments, the pharmaceutically acceptable carrier is sterile and pyrogen-free water. In some embodiments, the pharmaceutically acceptable carrier is Ringer's Lactate, sometimes known as lactated Ringer's solution.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Formulations of the present disclosure include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, tale, magnesium stearate, water, and mineral oil. The formulations can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

Local delivery by an implant describes the surgical placement of a matrix that contains the pharmaceutical agent into the affected site. The implanted matrix releases the pharmaceutical agent by diffusion, chemical reaction, or solvent activators.

For example, in some aspects, the disclosure is directed to a pharmaceutical composition comprising an active compound of Table 5, and a pharmaceutically acceptable carrier or diluent, or an effective amount of pharmaceutical composition comprising an active compound of Table 5.

The compounds of the present disclosure can be formulated for parenteral administration by injection, e.g., by bolus injection or continuous infusion. The compounds can be administered by continuous infusion subcutaneously over a predetermined period of time. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, alter adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragecanth, methyl cellulose, hydroxypropylmethyl-celllose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present disclosure can also be formulated in rectal compositions such as suppositories or retention enemas, e.g., containing conventional suppository bases such as cocoa butter or other glycerides.

In addition to the formulations described previously, the compounds of the present disclosure can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present disclosure, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

For parenteral administration, an analog can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of analog in 0.9% sodium chloride solution.

The present disclosure relates to routes of administration include intramuscular, sublingual, intravenous, intraperitoneal, intrathecal, intravaginal, intraurethral, intradermal, intrabuccal, via inhalation, via nebulizer and via subcutaneous injection. Alternatively, the pharmaceutical composition may be introduced by various means into cells that are removed from the individual. Such means include, for example, microprojectile bombardment and liposome or other nanoparticle device.

Solid dosage forms for oral administration include capsules, tablets, pills, powders and granules. In solid dosage forms, the analogs are generally admixed with at least one inert pharmaceutically acceptable carrier such as sucrose, lactose, starch, or other generally regarded as safe (GRAS) additives. Such dosage forms can also comprise, as is normal practice, an additional substance other than an inert diluent, e.g., lubricating agent such as magnesium state. With capsules, tablets, and pills, the dosage forms may also comprise a buffering agent. Tablets and pills can additionally be prepared with enteric coatings, or in a controlled release form, using techniques know in the art.

Liquid dosage forms for oral administration include pharmaceutically acceptable emulsions, solutions, suspensions and syrups, with the elixirs containing an inert diluent commonly used in the art, such as water. These compositions can also include one or more adjuvants, such as wetting agent, an emulsifying agent, a suspending agent, a sweetening agent, a flavoring agent or a perfuming agent.

One of skill in the art will recognize that the appropriate dosage of the compositions and pharmaceutical compositions may vary depending on the individual being treated and the purpose. For example, the age, body weight, and medical history of the individual patient may affect the therapeutic efficacy of the therapy. Further, a lower dosage of the composition may be needed to produce a transient cessation of symptoms, while a larger dose may be needed to produce a complete cessation of symptoms associated with the disease, disorder, or indication. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response. Dosages may also depend on the strength of the particular analog chosen for the pharmaceutical composition.

The dose of the composition or pharmaceutical compositions may vary. The dose of the composition may be once per day. In some embodiments, multiple doses may be administered to the subject per day. In some embodiments, the total dosage is administered in at least two application periods. In some embodiments, the period can be an hour, a day, a month, a year, a week, or a two-week period. In an additional embodiment of the invention, the total dosage is administered in two or more separate application periods, or separate doses over the course of an hour, a day, a month, a year, a week, or a two-week period.

In some embodiments, the one or plurality of active agents is one or a combination of compounds chosen from: an immunomodulating agent, a hormone agent, an anti-inflammatory compound, alpha-adrenergic agonist, analgesic compound, and an anesthetic compound. Non-limiting examples of such compounds are shown in Table 5 below.

TABLE 5
Examples of immunomodulating agents include:
Azficel-T
Etanercept
Glatiramcr
Lenalidomide
Mifamurtide
Pimecrolimus
Thymalfasin
Tinocordin
6Mercaptopurine
6MP
Actemra
Alferon N
anakinra
Arcalyst
Avonex
AVOSTARTGRIP
Berinert
Betascron
BG-12
C1 esterase inhibitor recombinant
C1 inhibitor human
Cinryze
Copaxone
dimethyl fumarate
ecallantide
Extavia
fingolimod
Firazyr
Gilenya
glatiramer
icatibant
immunoglobulins
Infergen
interferon alfa n3
interferon alfacon 1
interferon beta 1a
interferon beta 1b
Kalbitor
Kineret
mercaptopurine
peginterferon beta-1a
Plegridy
Purinethol
Purixan
Rebif
Rebif Rebidose
rilonacept
Ruconest
siltuximab
Sylvant
Tecfidera
tocilizumab
Examples of hormone agents include:
Estradiol
Synthetic conjugated estrogens
Estradiol valerate
Estradiol acetate
Estradiol estrogen
Estropipate
Conjugated estrogens
Progesterone
Micronized progesterone
Medroxyprogesterone
Medroxyprogesterone acetate
Norethindrone Acetate
Drospirenone
Levonorgestrel
Ethinyl Estradiol
Norgestimate
Bazedoxifene
GnRH agonists
Danazol
Testosterone
Examples of anti-inflammatory compounds include:
Aspirin
Celecoxib
diclofenac
diflunisal
etodolac
heparin
ibuprofen
indomethacin
ketoprofen
ketorolac nabumetone
naproxen
oxaprozin
piroxicam
prednisone
salsalate
sulindac
tolmetin
Examples of alpha-adrenergenic agonists include:
Methoxamine
Methylnorepinephrinc
Midodrine
Oxymetazoline
Metaraminol
Phenylephrine
Clonidinc (mixed alpha2-adrenergic and imidazoline-II receptor agonist)
Guanfacine, (preference for alpha2A-subtype of adrenoceptor)
Guanabenz (most selective agonist for alpha2-adrenergic as opposed
to imidazoline-II)
Guanoxabenz (metabolite of guanabenz)
Guanethidinc (peripheral alpha2-receptor agonist)
Xylazinc
Tizanidine
Medetomidine
Methyldopa
Fadolmidine
Dexmedetomidine
Examples of analgesic compounds include:
codeine
hydrocodone (Zohydro ER)
oxycodone (OxyContin, Roxicodone)
methadone
hydromorphone (Dilaudid, Exalgo)
morphine (Avinza, Kadian, MSIR, MS Contin), and
fentanyl (Actiq, Duragesic)
Examples of anesthetic compounds include:
Desflurane
Isoflurane
Nitrous oxide
Sevoflurane
Xenon

The compounds of the present disclosure can also be administered in combination with other active ingredients, such as, for example, adjuvants, or other compatible drugs or compounds where such combination is seen to be desirable or advantageous in achieving the desired effects of the methods described herein.

### System and Arrays

In many embodiments, an array comprises a solid support to whose surface(s) PIF and/or analogs thereof and/or other peptides or molecules are affixed in spatially discrete locations. Such an array can be prepared using PIF and/or analogs thereof from any source (e.g., recombinantly produced, biochemically isolated, synthetically made, commercially purchased, etc). Moreover, identity and relative amounts of individual peptide components may be determined or adjusted in accordance with requirements of a particular project or interests of a particular researcher.

For example, in many embodiments, it will be desirable to design, prepare and/or utilize an array that includes as many different PIF and/or analogs thereof as is feasible. Alternatively or additionally, in some embodiments, it may be desirable to design, prepare, and/or utilize an array that includes only peptides components known to be associated with (or not associated with) a particular cell or cell type or disorder, such as pregnancy, endometriosis, or RPL. To give a few particular examples, in some embodiments, an array is utilized that contains at least 1, 2, 3, 4, 5, 6, 7, 8, 9, 10, 11, 12, 13, 14, 15, 16, 17, 18, 19, 20 or more different “spots” (physically discrete locations) containing one or a plurality of different peptide components. In some embodiments, an array is utilized that contains from about 1 to about 100,000 spots, from about 100 to about 10,000, or from about 1,000 to about 5,000 spots.

In some embodiments, spots on an array comprise spatial organization. In some embodiments, spots on an array are arranged in a grid. In some embodiments, the array is arranged in a repetitive grid such that a plurality of grids are used to run multiple experiments with the same experimental variability simultaneously such that statistical significance can be determined.

In some embodiments, a variety of PIF or PIF analogs or combinations thereof are represented in spots of an array with each spot corresponding to both a known location on the array and a known composition of components. In certain embodiments, at least one component is spotted upon the array. In certain embodiments, the components are spotted individually. In some embodiments, mixtures of several peptide or analog components are contained within a single spot. In some embodiments, an array for use in accordance with the present invention includes both spots of single components and spots of combinations of components. In some embodiments, components are spotted multiple times in the same array, so that the array includes replicate spots. In some embodiments, an array for use in accordance with the present invention contains spots that lack a particular PIF peptide or analog thereof, and therefore may, for example, be utilized as negative controls in addition to spots containing PIF peptide or analogs thereof. In certain embodiments, rhodamine dextran is included in a negative control spot.

An array for use in accordance with the present invention may be prepared on any suitable substrate material. In many embodiments, the material will support viability and/or growth of cells, e.g., mammalian cells. In some embodiments, an array utilizes a substrate material selected from the group consisting of polyamides, polyesters, polystyrene, polypropylene, polyacrylates, polyvinyl compounds (e.g. polyvinylchloride), polycarbonate, polytetrafluoroethylene (PTFE), nitrocellulose, cotton, polyglycolic acid (PGA), cellulose, dextran, gelatin, glass, fluoropolymers, fluorinated ethylene propylene, polyvinylidene, polydimethylsiloxane, polystyrene, silicon substrates (such as fused silica, polysilicon, or single silicon crystals), and the like, or combinations thereof. Alternatively or additionally, metals (gold, silver, titanium films) can be used. In a some embodiments, acrylic slides coated with polyacrylamide are used. In some embodiments, an array utilizes one or a plurality of substrate materials that support a binding event between a peptide component of a spot (such as PIF or a PIF analog) and a cell or a protein expressed by a cell. In a some embodiments, acrylic slides coated with polyacrylamide are used. In some embodiments, an array utilizes one or a plurality of substrate materials that support a binding event between a peptide component of a spot (such as PIF or a PIF analog) and a cell or a protein expressed by a cell in a sample.

In some embodiments, the present invention provides arrays for use in culturing cells. In some embodiments, the arrays for use in culturing cells are provided with medium. In some embodiments, the arrays for use in culturing cells are provided with a sufficient volume of medium to support cell culture for 1, 2, 3, 4, 5 or more days.

In some embodiments, the present invention provides arrays for use as diagnostic assays. In some embodiments the arrays are provided as part of a diagnostic kit or detection kit. In some embodiments the arrays are provided as part of a detection kit. In certain embodiments, kits for use in accordance with the present invention may include one or more reference samples; instructions (e.g., for processing samples, for performing tests, for interpreting results, etc.); media; and/or other reagents necessary for performing tests.

In some embodiments, the system comprises at least one array comprising a solid support comprising at least one PIF peptide or analog thereof to the solid support, wherein the array comprises at least two or more polypeptides each comprising a polypeptide sequence associated with immune dysregulation, or an analog thereof chosen from the polypeptides of any of the tables provided herein. In some embodiments, the system comprises at least one array comprising a solid support wherein the solid support comprises: one or a plurality of PIF peptide and/or analogs thereof immobilized to a surface and at least two or more polypeptides each comprising a polypeptide sequence associated with immune dysregulation, endometriosis, recurrent pregnancy loss, or pregnancy or an analog thereof chosen from any of the peptides disclosed herein; wherein the solid support comprises a material chosen from: polysterene (TCPS), glass, quarts, quartz glass, poly(ethylene terephthalate) (PET), polyethylene, polyvinyl difluoride (PVDF), polydimefhylsiloxane (PDMS), polytetrafluoroethylene (PTFE), polymethylmethacrylate (PMMA), polycarbonate, polyolefin, ethylene vinyl acetate, polypropylene, polysulfone, polytetrafluoroethylene, silicones, poly(meth)acrylic acid, polyamides, polyvinyl chloride, polyvinylphenol, and copolymers mixtures thereof.

In some embodiments, the system comprises at least one array comprising a solid support, prepared by the steps comprising: (i) preparing a first and second solution, each first and second solution comprising a known concentration of a polypeptide comprising a polypeptide sequence associated with the a polypeptide sequence associated with immune dysregulation, endometriosis, RPL, or pregnancy or an analog thereof; (ii) contacting the first and second solution with the solid support for a sufficient time period absorb polypeptide comprising a polypeptide sequence or analog thereof associated with immune dysregulation, endometriosis, RPL, or pregnancy to the solid support; wherein the polypeptide sequence associated with a polypeptide sequence associated with immune dysregulation, endometriosis, recurrent pregnancy loss, or pregnancy or an analog thereof is chosen from the polypeptides of Table 1 or Table 4; and wherein the steps of preparing a solution and contacting the solution with the solid support is repeated at least about 1,10, 20, 30, 40, 50, 60, 70, 80, 90, 100, 110, 120, 130, 140, 150, 160, 170, 180, 190, 200, 300, 400, 500, 600 or 700 times corresponding to the number of spots or discrete locations present on the at least one array. In some embodiments, the one or more repeated steps of contacting the first and second solution with the solid support is performed by an automated device such that each polypeptide comprising a polypeptide sequence or analog thereof associated with immune dysregulation, endometriosis, RPL, or pregnancy is absorbed at discrete addressable locations on the at least one array.

According to some embodiments, the array comprises a chip or silica surface coated with a metal such as silver configured for use within a device that measures surface plasmon resonance or (SPR). In some embodiments the chip is a BIAcore chip (furnished by GE life Sciences), such as a CM5 chip. The sensor chip is fixed to a polystyrene support frame in a protective sheath. Each cassette, consisting of a sensor chip and sheath assembly, is individually packed under a nitrogen atmosphere in a hermetically sealed pouch.

The BIAcore chip can be used according to the manufacturer's instructions (found at https://www. gelifesciences.com/gehcls_images/GELS/Related%20Content/Files/1443019450 961/litdoc22031023_20150923164404.pdf, which is incorporated by reference in its entirety) but, briefly one of ordinary skill would know that the CM5 chip, as a non-limitative example, comprises cyclomethyldextran on its surface onto which one or a plurality of polypeptides or analogs disclosed herein may be immobilized through known chemistry. Briefly, the protocol comprises one or more of the following steps:

(a) Allow the sealed sensor chip pouch to equilibrate at room temperature for 15 to 30 minutes in order to prevent condensation on the chip surface; (b) prepare the Biacore instrument with known running buffer. The buffer should be filtered (0.22 urn), and degassed for systems that do not have an integrated buffer degasser; (c) open the sensor chip pouch. Make sure that the sensor chip support remains fully inserted into the sheath at all times, (d) dock the sensor chip in the instrument as described in the Instrument Manual Handbook; (e) sensor chips that are not docked in the instrument should be stored in closed containers.
Immobilizing the polypeptide or analog thereof:
   The ligand or capturing molecule is covalently bound to the sensor chip surface via carboxyl groups on the dextran. Functional groups on the molecule that can be used for coupling include —NH2, —SH, —CHO, —OH and —COOH. The surface is prepared by For most immobilization approaches, the carboxymethyldextran surface is activated with a mixture of 1-ethyl-3-(3-dimethylaminopropyl)carbodiimide (EDC) and N-hydroxysuccinimide (NHS). Reagent solutions should be freshly prepared and mixed shortly before use. The efficiency of immobilization will be reduced if the solutions are not fresh.

According to some embodiments, the array comprises a formulation may be supplied as part of a kit. In some embodiments, the kit comprises comprising a PIF peptide and/or a PIF analog or pharmaceutically acceptable salt thereof, the PIF peptide and/or a PIF analog or pharmaceutically acceptable salt thereof comprises a non-natural amino acid or is at least 70% homologous to SEQ ID NO:20. In another embodiment, the kit comprises a pharmaceutically acceptable salt of an analog with a rehydration mixture. In another embodiment, the pharmaceutically acceptable salt of an analog are in one container while the rehydration mixture is in a second container. The rehydration mixture may be supplied in dry form, to which water or other liquid solvent may be added to form a suspension or solution prior to administration. Rehydration mixtures are mixtures designed to solubilize a lyophilized, insoluble salt of the invention prior to administration of the composition to a subject takes at least one dose of a purgative. In another embodiment, the kit comprises a pharmaceutically acceptable salt in orally available pill form.

In some embodiments, the kit comprises at least one array comprising a solid support comprising at least one PIF peptide or analog thereof to the solid support; wherein the array comprises at least two or more polypeptides each comprising a polypeptide sequence associated with immune dysregulation, or an analog thereof chosen from the polypeptides of Table 4, as described above.

The kit may contain two or more containers, packs, or dispensers together with instructions for preparation and immobilization. In some embodiments, the kit comprises at least one container comprising the pharmaceutical composition or compositions described herein and a second container comprising a means for delivery of the compositions such as a syringe. In some embodiments, the kit comprises a composition comprising an analog in solution or lyophilized or dried and accompanied by a rehydration mixture. In some embodiments, the analog and rehydration mixture may be in one or more additional containers.

The compositions included in the kit may be supplied in containers of any sort such that the shelf-life of the different components are preserved, and are not adsorbed or altered by the materials of the container. For example, suitable containers include simple bottles that may be fabricated from glass, organic polymers, such as polycarbonate, polystyrene, polypropylene, polyethylene, ceramic, metal or any other material typically employed to hold reagents or food; envelopes, that may consist of foil-lined interiors, such as aluminum or an alloy. Other containers include test tubes, vials, flasks, and syringes. The containers may have two compartments that are separated by a readily removable membrane that upon removal permits the components of the compositions to mix. Removable membranes may be glass, plastic, rubber, or other inert material.

Kits may also be supplied with instructional materials. Instructions may be printed on paper or other substrates, and/or may be supplied as an electronic-readable medium, such as a floppy disc, CD-ROM, DVD-ROM, zip disc, videotape, audio tape, or other readable memory storage device. Detailed instructions may not be physically associated with the kit; instead, a user may be directed to an internet web site specified by the manufacturer or distributor of the kit, or supplied as electronic mail

In another embodiment, a packaged kit is provided that contains the pharmaceutical formulation to be administered, i.e., a pharmaceutical formulation comprising PIF peptide and/or a PIF analog or pharmaceutically acceptable salt thereof, a container (e.g., a vial, a bottle, a pouch, an envelope, a can, a tube, an atomizer, an aerosol can, etc.), optionally a solid support, optionally sealed, for housing the formulation during storage and prior to use, and instructions for carrying out drug administration in a manner effective to treat any one or more of the indications disclosed herein. The instructions will typically be written instructions on a package insert, a label, and/or on other components of the kit.

Depending on the type of formulation and the intended mode of administration, the kit may also include a device for administering the formulation (e.g., a transdermal delivery device). The administration device may be a dropper, a swab, a stick, or the nozzle or outlet of an atomizer or aerosol can. The formulation may be any suitable formulation as described herein. For example, the formulation may be an oral dosage form containing a unit dosage of the active agent, or a gel or ointment contained within a tube. The kit may contain multiple formulations of different dosages of the same agent. The kit may also contain multiple formulations of different active agents.

The present kits will also typically include means for packaging the individual kit components, i.e., the peptide forms (immobilized or not immobilized), an administration device (if included), a solid support for immobilization of the peptides disclosed herein or a solid support comprising the immobilized peptides disclosed herein and the written instructions for use. Such packaging means may take the form of a cardboard or paper box, a plastic or foil pouch, etc.

### Methods

Embodiments of the disclosure are directed to methods of examining PIF binding to a subject's circulating immune cells as a marker for immune dysregulation. Some embodiments are directed to a method of identifying a female subject with a history of RPL due to immune dysregulation comprising administering an effective amount of PIF, and examining its binding to circulating immune cells. Within that method, a deviation from normal values of PIF binding to circulating immune cells compared to a reference indicates that the subject's history of RPL is likely due to immune dysregulation, whereas normal binding of PIF to circulating immune cells compared to a reference indicates that the subject's history of RPL is likely not due to immune dysregulation. In some embodiments, the subject's circulating immune cells are DCs. In certain embodiments, the DCs are pDCs, mDCs, or combinations thereof.

Other embodiments are directed to a method of identifying a female subject likely to suffer from RPL due to immune dysregulation, comprising administering an effective amount of PIF, and examining its binding to circulating immune cells. Within that method, a reduction of PIF binding to circulating immune cells compared to a reference indicates that the subject is likely to suffer from RPL due to immune dysregulation, and normal binding of PIF to circulating immune cells compared to a reference indicates that the subject is not likely to suffer from RPL due to immune dysregulation. In some embodiments, the subject's circulating immune cells are DCs. In certain embodiments, the DCs are pDCs, mDCs, or combinations thereof.

Other embodiments are directed to a method of identifying a subject with immune dysregulation, comprising administering an effective amount of PIF, and examining its binding to circulating immune cells. Other embodiments are directed to a method of identifying a subject with immune dysregulation comprising administering an effective amount of PIF or an analog thereof, and analyzing binding of the PIF to circulating immune cells. Within that method, a reduction of PIF binding to circulating immune cells compared to a reference indicates the subject's immune dysregulation, and normal binding of said PIF to said circulating immune cells compared to a reference indicates the subject's lack of immune dysregulation. In some embodiments, the subject's circulating immune cells are DCs. In certain embodiments, the DCs are pDCs, mDCs, or combinations thereof.

Other embodiments are directed to a method of identifying a subject with endometriosis, comprising administering an effective amount of PIF, and examining its binding to circulating immune cells. Within that method, a reduction of PIF binding to circulating immune cells compared to a reference indicates the subject's endometriosis, and normal binding of said PIF to said circulating immune cells compared to a reference indicates the subject's lack of endometriosis. In some embodiments, the subject's circulating immune cells are DCs. In certain embodiments, the DCs are pDCs, mDCs, or combinations thereof.

In some embodiments, a method of identifying a subject with immune dysregulation may comprise exposing an effective amount of PIF or an analog thereof to a sample from the subject comprising one or a plurality of immune cells, and examining a binding event between the one or among a plurality of immune cells of the subject and PIF or an analog thereof; wherein a significant change of binding of PIF to the one or plurality of immune cells as compared to a reference indicates that the subject has immune dysregulation.

In some embodiments, a binding event may be examined, determined, measured, or characterized by an assay. In some embodiments, the assay may be, for example, an enzyme-linked immunosorbent assay (ELISA), flow cytometry, or affinity chromatography. In some embodiments, PIF binding may be determined using a sensor such as, for example, a biosensor.

Generally, ELISA protocols begin with a capture antibody, specific for a protein of interest, coated onto the wells of microplates. Samples, including a standard containing protein of interest, control specimens, and unknowns, are pipetted into these wells. During the first incubation, the protein antigen binds to the capture antibody. After washing, a detection antibody is added to the wells, and this antibody binds to the immobilized protein captured during the first incubation. After removal of excess detection antibody, an HRP conjugate (secondary antibody or streptavidin) is added and binds to the detection antibody. After a third incubation and washing to remove the excess HRP conjugate, a substrate solution is added and is converted by the enzyme to a detectable form (color signal). The intensity of this colored product is directly proportional to the concentration of antigen present in the original specimen. An ELISA is used to quantify antigens. ELISAs are adaptable to high-throughput screening because results are rapid, consistent and relatively easy to analyze. Results can be obtained with the sandwich format, utilizing highly purified, pre-matched capture and detection antibodies. The resulting signal provides data which is very sensitive and highly specific. Ready-to-use ELISA kits are commercially available for hundreds of commonly investigated proteins and other biological molecules.

Generally, flow cytometry is a process that allows for the individual measurements of cell fluorescence and light scattering. Such measurements are performed at rates of thousands of cells per second, and the resulting information can be used to individually sort or separate subpopulations of cells. Briefly, cells are loaded onto the collection stage of the flow cytometer. The sample is drawn up into the fluidic system and pumped to the flow chamber, or flow cell. The cells are combined with a stream of sheath fluid which quickly moves them, one at a time, past one or more light sources (for example, lasers). The beam of light from the laser excites the cells as they pass through the flow chamber. Light scattering and/or fluorescence are captured, filtered spectrally, and converted to electrical signals (voltage) through photodetectors. An external computer system then digitizes the voltage data. The digital information is analyzed to quantitate the characteristics of the cells. Flow cytometry may be particularly useful for the high-speed analysis of one or more samples. In some instances, flow cytometry may involve washing cells twice in sterile PBS and lysing any unwanted cells with 0.16 M ammonium chloride solution. Immune cells may be incubated with 1, 5 or 10 ug/ml FITC-PIF or FITC-PIFscr for 1 hour at room temperature, then washed three times to remove un-bound peptide and fixed for flow cytometry. Cell types may be separated based upon their scatter characteristics. Publication no. WO/2009/045443, which is hereby incorporated by reference in its entirety, provides additional information about methods of obtaining flow cytometry data.

Generally, affinity chromatography is a powerful chromatographic method for purifying a specific molecule or a group of molecules from complex mixtures. It is based on highly specific biological interactions between two molecules, such as interactions between enzyme and substrate, receptor and ligand, or antibody and antigen. These interactions, which are typically reversible, are used for purification by placing one of the interacting molecules, referred to as affinity ligand, onto a solid matrix to create a stationary phase while the target molecule is in the mobile phase. The molecule of interest will typically have a well-known and defined property, which can be exploited during the affinity purification process. The process itself can be thought of as trapping the target molecule on a solid or stationary phase or medium. The other molecules in the mobile phase will not become trapped, as they do not possess this property. The stationary phase can then be removed from the mixture, washed, and the target molecule released from the entrapment in a process known as elution. In some instances, affinity chromatography may involve producing a purified protein of interest using an affinity chromatography (AC) matrix to which the protein of interest is bound, by loading a mixture comprising the protein of interest onto the AC matrix; washing the AC matrix with a wash solution comprising arginine, or an arginine derivative, at a pH greater than 8.0; and eluting the protein of interest from the AC matrix, wherein the wash is performed without the presence of a nonbuffering salt. Publication no. WO/2012/164046, which is hereby incorporated by reference in its entirety, provides additional information about methods of completing affinity chromatography.

In some embodiments, PIF may be associated with a solid support and one or a plurality of PIF peptides or analogs thereof, wherein the one or a plurality of PIF peptides or analogs are attached to the solid support at an addressable location of an array. In some embodiments, the solid support is a slide optionally coated with a polymer. In some embodiments, the solid support is coated with a polymer. In some embodiments, the polymer is polyacrylamide. In some embodiments, the solid support is a material chosen from: polysterene (TCPS), glass, quarts, quartz glass, poly(ethylene terephthalate) (PET), polyethylene, polyvinyl difluoride (PVDF), polydimethylsiloxane (PDMS), polytetrafluoroethylene (PTFE), polymethylmethacrylate (PMMA), polycarbonate, polyolefin, ethylene vinyl acetate, polypropylene, polysulfone, polytetrafluoroethylene, silicones, poly(meth)acrylic acid, polyamides, polyvinyl chloride, polyvinylphenol, and copolymers and mixtures thereof. In some embodiments, the at least one adhesion set comprises two different polypeptides attached to a solid support.

In some embodiments, PIF binding may be compared to a standard, or reference, binding profile. In some embodiments, the reference binding profile serves as a comparison for testing PIF binding to PBMC subtypes. In healthy subjects, FITC-PIF binds ˜100% of CD14+ cells and <10% to T, B, and NK cells when exposed to low 300-500 nM in normal patients. A >20% decrease in binding to CD 14+ cells and a >20% increase in binding to T, B, and NK cells in non-pregnant subjects following the same exposure to FITC-PIF constitutes a risk for RPL or immune dysregulation. PIF binding to PBMCs significantly increases during pregnancy, and following exposure to mitogens or immune activators. The inability to bind 100% of CD 14+ cells in naive cells, or the inability to increase binding following activation, reflects immune dysfunction as seen in disorders including but not limited to RPL and endometriosis. A decrease in PIF binding to CD 14+ cells compared to normal may indicate that a subject's innate immunity is affected. A >20% increase in PIF binding to cells selected from T, B, NK cells and combinations thereof (PBMCs) compared to normal may indicate that a subject's adaptive immunity is affected. Accordingly, in some embodiments, the blood samples are collected from patients, the PBMCs are separated using Ficoll-Hypaque, and the binding profile of the separated PBMCs is examined. FITC-PIF (500 nM) is exposed to the PBMCs for 30 min in culture media (RPMI serum-free) at RT. Subsequently, the PBMCs are washed to remove excess FITC-PIF, and the labeled PBMCs are placed in a flow cytometer to analyze the interaction with various immune phenotypes by using specific anti-CD3, CD4, CD8, CD 19, and CD56 antibodies in 203 colors. Specific binding is determined in gated quadrants. The reference binding profile is wherein PIF binds to about 100% of CD14+ cells (i.e., monocytes and/or macrophages) and binds to less than about 10% of CD4, CD8 and/or B cells. Therefore, in some embodiments, immune dysregulation is identified when PIF binds to only ˜80% of CD14+ cells (i.e., monocytes and/or macrophages) and binds to >20% of CD4, CD8 and/or T, B, and NK cells. In alternative embodiments, FITC-PIF is added at a higher concentration of about 25 uM. At that higher concentration, different binding results are expected in subjects with various forms of immune dysregulation. In particular, at the higher concentration, the binding of PIF to CD4, CD8, and/or T, B, and NK cells is expected to increase in normal subjects; thus, a lack of increase in binding, or failure of the binding to increase, indicates the subject's immune dysfunction. In other alternative embodiments, PIF binding is examined in the presence of PHA, wherein binding to CD4+, CD8+, and CD19+ cells is expected to decrease approximately 30-fold. Therefore, in the presence of PHA, the failure of PHA binding to increase as expected indicates the subject's immune dysfunction. Herein, the terms “reference,” “control,” “standard,” “average,” and the like refer generally to the normal binding characteristics described above.

In some embodiments, methods of the disclosure comprise measuring, analyzing or comparing a significant change. In some embodiments, a “significant change” refers to a statistically significant result. Generally, statistical significance (or a statistically significant result) is attained when a p-value is less than the significance level. The p-value is the probability of obtaining at least as extreme results given that the null hypothesis is true, whereas the significance or alpha (a) level is the probability of rejecting the null hypothesis given that it is true. A significance level chosen before data collection may be, for example, 0.05 (5%). In some embodiments, a Student's t-test may be used to assess significance. Generally, a t-test is any statistical hypothesis test in which the test statistic follows a Student's t-distribution if the null hypothesis is supported. It can be used to determine if two sets of data are significantly different from each other, and is most commonly applied when the test statistic would follow a normal distribution if the value of a scaling term in the test statistic were known. When the scaling term is unknown and is replaced by an estimate based on the data, the test statistic (under certain conditions) follows a Student's t distribution. In some embodiments, other statistical tests may be used to determine significance. In some embodiments, In some embodiments, the PIF peptide may be used to test its binding to different immune phenotypes. In some embodiments, such PIF peptide binding may be compared in pregnant and non-pregnant subjects. In some embodiments, a difference between such PIF binding compared to a reference may be expressed as a mean +/−standard error of the mean (SEM) or standard deviation (SD). In some embodiments, a difference between such PIF binding compared to a reference may be expressed as 2 standard deviations. In some embodiments, the PIF peptide may be used to measure PIF's effect on immune cell function, wherein subjects with a history of RPL are compared to a reference. In come embodiments, the immune cell function may be determined by examining changes in cytokine secretion. In some embodiments, the significant change may be +/−about 20%.

In some embodiments, the PIF peptide may be used to measure whether it is affected by sera from one or more subjects with a history of endometriosis. In some embodiments, the significant change may be +/−about 20%.

In some embodiments, the PIF peptide is selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or analogs thereof, and combinations thereof. In certain embodiments, the PIF peptide is selected from SEQ ID NO: 1; SEQ ID NO: 2; SEQ ID NO: 3; SEQ ID NO: 4, and combinations thereof. In the some embodiments, the PIF peptide may be selected from compounds having amino acid structural and functional analogs, for example, peptidomimetics having synthetic or non-natural amino acids or amino acid analogues, so long as the mimetic has one or more functions or activities of compounds of the disclosure.

In some embodiments, the PIF peptide may be used to test its binding to CD45+ cells in non-pregnant mice. In some embodiments, the PIF peptide may be used to determine PIF targets in vivo by assessing FITC-PIF targets. In some embodiments, the targets may be, for example, spleen or bone marrow.

In some embodiments, any of the methods disclosed herein comprise a step of isolating or taking a sample from a subject. In some embodiments, any of the methods disclosed herein comprise exposing a sample to high performance liquid chromatography (HPLC) prior to examining or analyzing or measuring a binding event or binding affinity between PIF or an analog thereof and one or more cells. In some embodiments, any of the methods disclosed herein comprise exposing a sample to PIF or an analog thereof to one or a plurality of cells, either isolated or as a component in a sample, before isolating the one or plurality of cells and creating a binding profile based upon the protein expression of the one or plurality of cells. In some embodiments, the methods comprise immune cells such as isolated bone marrow cells, splenic cells, PBMCs, CD45+ cells, CD14+ cells, CD4+ cells, CD8+ cells, dendritic cells, CD25+ cells, FoxP3+ cells, CD4+/CD25+/FoxP3+ cells. And, in some embodiments, the protein expression measured comprises measuring or analyzing the amount of cytokines expressed by the one or plurality of isolated cells.

In some embodiments, any of the methods disclosed herein comprise a step of analyzing the amount of protein bound to the one or plurality of cells by quantifying the amount of dye or fluorescence from a dye or other detection moiety covalently or non-covalently bound to the protein. A binding event may be visualized, detected or quantified using any technique known in the art to bind to a polypeptide, such as PIF or an analog thereof. In some embodiments, the immobilized protein such as PIF or an analog thereof may comprise a detection moiety that enables intercalating, covalent or non-covalent binding, or adsorption of a dye or other label that facilitates visualization or quantification of an amount of polypeptide used in any method. Examples of labels of polypeptides useful for any of the methods herein are as follows: a singlet oxygen radical generator such as resorufin, malachite green, fluorescein, FITC or diaminobenzidine; an analyte-binding group, such as a metal chelator, non-limiting examples of which include: EDTA, EGTA, a pyridinium, an imidazole and a thiol; a heavy atom carrier, such as iodine; an affinity tag such as a histidine tag, a GST tag, a FLAG tag and an HA tag; photoactivatable cross-linkers such as benzophenones and aziridines; a photoswitch label such as azobenzene; and a photolabile protecting group such as a nitrobenzyl group, a dimethoxy nitrobenzyl group or NVOC, or large macromolecules such as antibodies specific to a polypeptide disclosed herein comprising a tag or label (those used for immunohistochemistry experiments disclosed herein are one non-limiting example). In some embodiments, any of the methods disclosed herein comprise a step of analyzing the amount of protein bound to the one or plurality of cells by quantifying the amount of dye or fluorescence from a dye or other detection moiety covalently or non-covalently bound to the protein by stimulating the excitation of the label or detection moiety with an electromagnetic wave. For example, in the case of photolabile detection moieties, the chemical moiety bound to PIF or other polypeptide may be exposed to light which cleaves the chemical moiety from a protein in a concentration-dependent fashion. The amount of reaction product in a sample can be correlated with the amount of signal obtained corresponding to the reaction product.

The disclosure further relates to a method of diagnosing immune dysregulation in a subject comprising: (a) contacting a cell sample to an array or system disclosed herein; (b) quantifying one or more binding events; (c) determining one or more binding signatures of the cell sample based upon the binding events; and (d) comparing the binding signature of the cell sample to a binding signature of a control cell sample. The disclosure also provides a method of isolating a cell comprising: contacting a cell sample to an array or system disclosed herein. In some embodiments, the method of isolating a cell comprises contacting a cell sample to an array or system disclosed herein for a sufficient time period and under sufficient conditions for a cell to adhere to the array or the system more tightly than other components of the cell sample. In some embodiments, the method of isolating a cell further comprises rinsing the array or system with a buffer that that washes other non-binding components of the cell sample from the cell.

In some embodiments, any of the methods disclosed herein comprise a step of analyzing the amount of protein bound to the one or plurality of cells by quantifying the amount of dye or fluorescence from a dye or other detection moiety covalently or non-covalently bound to the protein in vivo after administration of one or a plurality of PIF peptides or analogs thereof into the subject. In some embodiments, any of the methods disclosed herein comprise exposing a sample to PIF or an analog thereof to one or a plurality of cells in vivo, before isolating the one or plurality of cells and creating a binding profile based upon the protein expression of the one or plurality of cells. In some embodiment, the analysis may be performed by digital microscopy, in some embodiments, the analysis comprises taken a section of biopsy and exposing the section to digital or light microscopy.

In some embodiments, the PIF peptide may be used to determine its binding to immune cells such as, for example, CD 14+, CD8+, or CD4+ cells, by conducting affinity chromatography followed by mass spectrometry analysis to identify proteins and compare binding among them by ranking concentration. In some embodiments, the PIF peptide may be used to determine its binding to immune cells such as, for example, CD14+, CD8+, or CD4+ cells, by conducting affinity chromatography followed by high performance liquid chromatography (HPLC), mass spectrometry analysis to identify proteins and compare binding among them by ranking concentration. In some embodiments, the results may be compared to abnormal PBMCs to determine whether the ranking of concentration amounts or quantification of protein expression changes, or whether there are different proteins or pathways involved.

In some embodiments, the PIF in serum from pregnant and non-pregnant horses may be compared. In some embodiments, the PIF in serum from pregnant and non-pregnant horses may be compared. In some embodiments, immobilized PIF binding to isolated cell may be expressed as mean +/−SEM. In some embodiments, cytokine levels in serum and placenta in healthy, PIF-treated, LPS-treated, and PIF+LPS-treated mice may be compared. In some embodiments, the results may be expressed as mean +/−SEM. In some embodiments, immune dysfunction may be diagnosed if there are significant changes in the values. For example, in some embodiments, a significant change may comprise a shift of more than about twice the SEM or SD of a mean result.

In some embodiments, cytokine levels in serum and placenta in healthy, PIF-treated, LPS-treated, and PIF+LPS-treated mice may be compared. In some embodiments, the results may be expressed as mean +/−SEM,

In some embodiments, immune dysfunction may be diagnosed if there are significant changes in the values. In any of the foregoing embodiments, a significant change may comprise a shift of more than about twice the SEM or SD of a mean result.

Any publications disclosed in this application (whether journal article or patent application or other publication) is incorporated herein in their entireties. This disclosure and embodiments illustrating the method and materials used may be further understood by reference to the following non-limiting examples.

### EXAMPLE 1

PIF plays an essential role during pregnancy, acting not only on local immunity but also systemically, as demonstrated by the immunomodulatory effects of sPIF on PBMCs. Naive CD 14+ monocytes are PIF's primary target.

The objective of our study was to investigate whether sPIF plays a role in generating tolerogenic DCs from peripheral blood (PB) monocytes. These findings would indicate the possible involvement of this peptide in generating systemic maternal immune tolerance.

A DC can be defined as tolerogenic by having a specific antigenic profile, and more importantly by its immunomodulatory functions (the ability to inhibit T-cell activation and to induce and promote regulatory T-cell development and expansion).

CD 14+ monocytes purified by immunomagnetic selection from healthy donor PB was cultured under serum-free conditions with different cytokine combinations in order to promote “classical” DC differentiation or putative tollerogenic differentiation.

Phenotypic characterization and functional tests were also performed on DCs isolated from the PB of women in their first and second trimesters of pregnancy.

The finding that PIF could be involved in the generation of tolerogenic DCs could further explain the immune changes that occur during pregnancy and autoimmune diseases.

PIF Binding to pDCs and mDCs as a Marker for Pregnancy Loss

PIF production throughout a viable pregnancy is necessary for the embryo to survive and condition the uterine environment. PIF also conditions the maternal immune system; synthetic PIF (sPIF) transposes the functions of natural PIF. Endometrial cells and cells of the monocyte/macrophage lineage are PIF's main targets. Through direct action, PIF acts as a rescue factor to prevent the demise of embryos cultured in the presence of serum from subjects with recurrent pregnancy loss (RPL). Moreover, PIF has been shown to reduce natural killer (NK) cell cytotoxicity in RPL subjects. Because the immune system either directly or indirectly plays a dominant role in RPL, and because dendritic cells (DCs) regulate immune responses, we compared the number and binding of exogenous PIF to circulating Th2-promoting plasmacytoid DCs (pDCs) and Th1 pro-inflammatory myeloid DCs (mDCs) in 13 RPL subjects and 11 healthy pregnant (HP) subjects.

Materials and reagents used: polypropilene tubes (Greiner) Lysing solution IX (BD Pharm Lyse), phosphate-buffered saline (PBS), Dulbecco A (Oxoid), PIF-1 FITC (lot AAF-192//387-66), anti-FITC (BD), anti-hCD123 PE (BD), anti-hCD1lc APC (BD), anti-hHLA-DR PerCP (BD), and BD FACSCanto.

Methods: 1OO uL of whole blood were incubated with 2mL of lysing solution for 10 min at room temperature (RT). Samples were washed with 2mL of PBS and centrifuged at 1200 rpm (break 1). Pellets were gently re-suspended in 1OO uL PBS and incubated with anti-CD123, -lineage cocktail, CD1 lc and HLA-DR antibodies for 15 minutes at RT in the dark. Samples were washed with 2 mL of PBS and centrifuged at 1200 rpm. Pellets were gently re-suspended in 1 mL PBS and incubated with 0.118 uM for 1 h at RT. Samples were washed with 2 mL of PBS and centrifuged at 1200 rpm. Samples were re-suspended in 500 uL PBS, and immediately run on a BD FACSCanto.

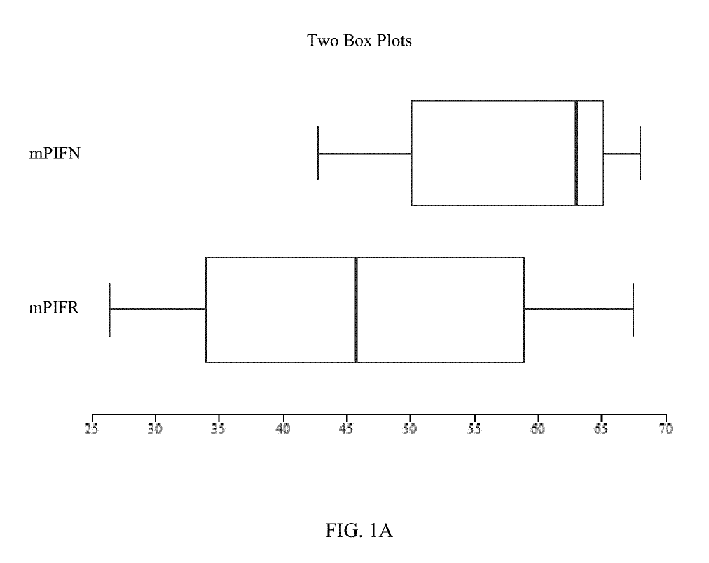

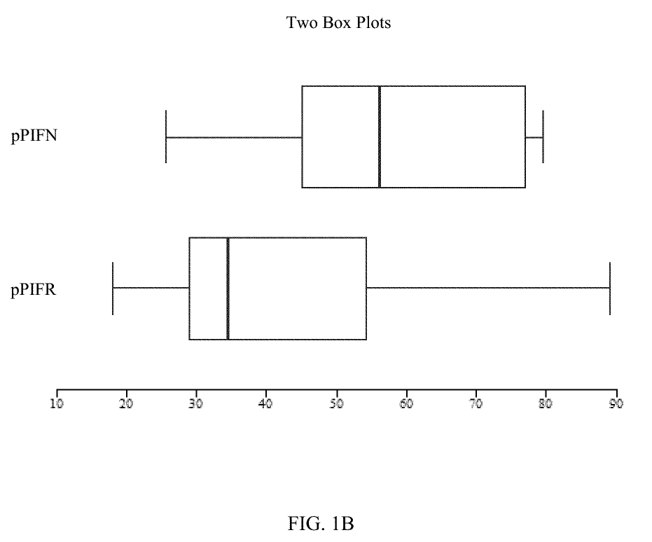

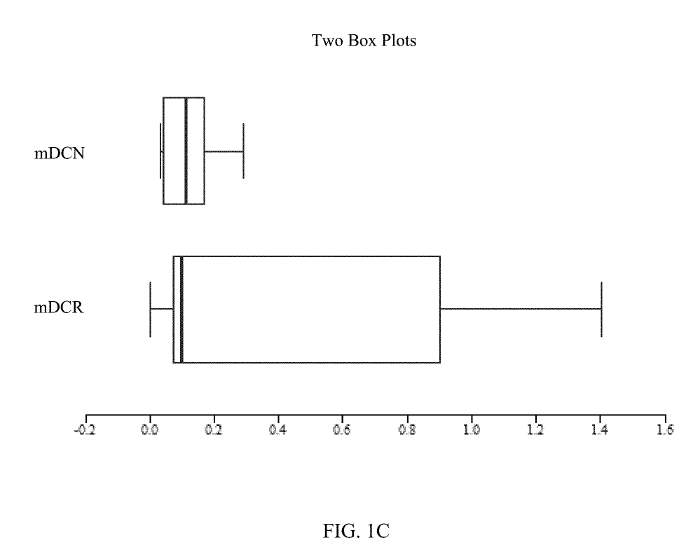

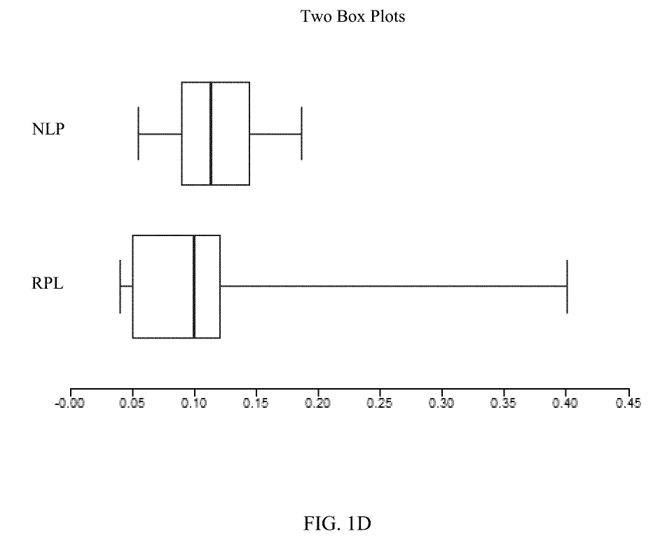

Results: 4 RPL subjects showed a >10-fold increase of mDCs, while 7 RPL subjects had values similar to the HP group (0.10±0.08); no difference in the percent of pDCs was observed (0.113+.0.09 in the RPL group vs. 0.116+0.03 in the HP group). Gestational age did not modify the value of either pDCs or mDCs in the HP group. PIF binding cells were reduced equally in pDCs and mDCs in the RPL group (pDC PIF+: 41.2±19.2 in the RPL group vs. 58.2±18.3 in the HP group, p=0.0381; mDC PIF+: 46.1+14.2 in the RPL group vs. 57.9±9.1 in the HP group; p=0.029). There was no relationship between the level of mDCs present in the individual RPL subject and the % of mDC PIF+ (FIGS. 1A-D). These data suggest that a reduction of PIF binding to DCs can represent a marker of RPL risk.

### EXAMPLE 2

Identification of Altered Immunity Prior to Pregnancy in a Case of RPL

A subject with a history of 18 miscarriages was studied to determine whether PIF could identify an immune defect in this subject as compared to a healthy non-pregnant subject. The binding of PIF to both naive and activated immune cells was examined Generally, prior to pregnancy, PIF binds primarily to CD 14+ cells; during pregnancy, however, the binding increases from 60-70% to 80-90% at high fluorescein isothiocyanate (FITC)-PIF exposure. Therefore, elevated binding prior to pregnancy indicates the pathologic activation of the immune cells. Such binding also affects peripheral blood mononuclear cells (PBMCs). This effect is exerted on naive cells where the effect on cytokine secretion is modeled while there are effects on several genes' expressions. In contrast, after activation by anti-CD3, CD3/CD28, LPS, and/or PHA, the immune response is greatly amplified. Therefore, the inappropriate response following the exposure of PBMCs to PIF reflects excessive immune activation prior to pregnancy, and may provide an index of potential pregnancy pathology or possible recurrent pregnancy loss (RPL).

A subject with a history of 18 miscarriages was studied to determine FITC-PIF binding. In addition, the effect of PIF on the percent of this subject's lymphocytes expressing a given cytokine was determined, and the results were compared to those of a healthy control. Furthermore, the same experiment was conducted by activating the PBMCs with a potent mitogen phytohemagglutinin (PHA) 1 μg/mL.

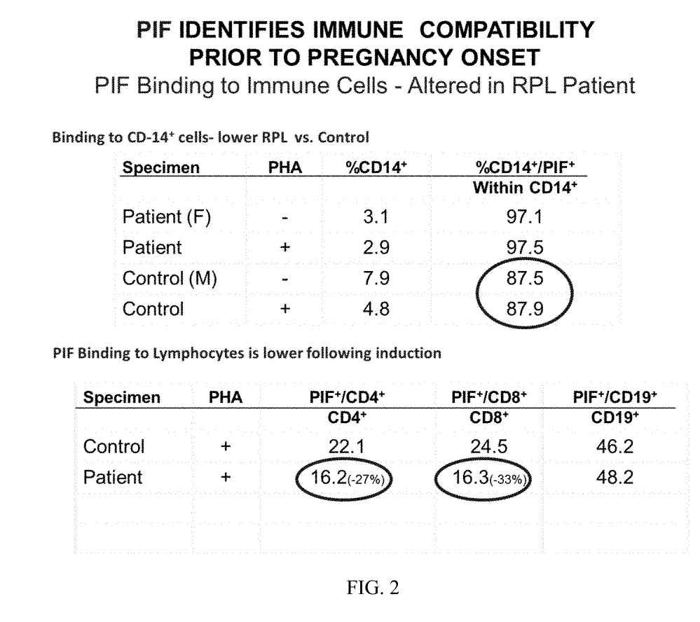

Normal donor PBMCs were washed and cultured (2.4×106 per well in a cluster of 24 wells) in AIM-V Medium with lug/mL PHA and 30 nM PIF or scrambled PIF (PIFscr). Medium was exchanged for fresh medium with PIF (without PHA) daily after day 2, until day 4 when the experiment was completed. Monensin and Berfeldin, 2 μM and 1 μg/mL, respectively, were added 6 hours before harvesting. Cells were mixed with surface marker-specific antibodies (CD4+), then processed for fixation and permeabilization per the manufacturer's protocol (Beckman-Coulter), and stained with cytokine-specific antibodies (anti-IFNγ or anti-IL10). Cells were analyzed on a Coulter Epics XL Flow Cytometer, using three-color analysis. Scatter-gating included both small and large (blast) lymphocytes, and all cytokine-positive cells were counted. Cells were exposed to 1.5 μg/mL FIC for 30 minutes, followed by washing off the non-attached ligand. Subsequently, the binding to PBMCs was determined by using two-color flow cytometry. Data showed that binding to CD 14+ cells was amplified compared to controls (FIG. 2). No difference was observed when cells were activated. When binding to other lineages in the presence of PHA was examined as compared to the control, the binding to both CD4 and CD8 decreased, while no difference in binding to CD19 was noted (FIG. 2).

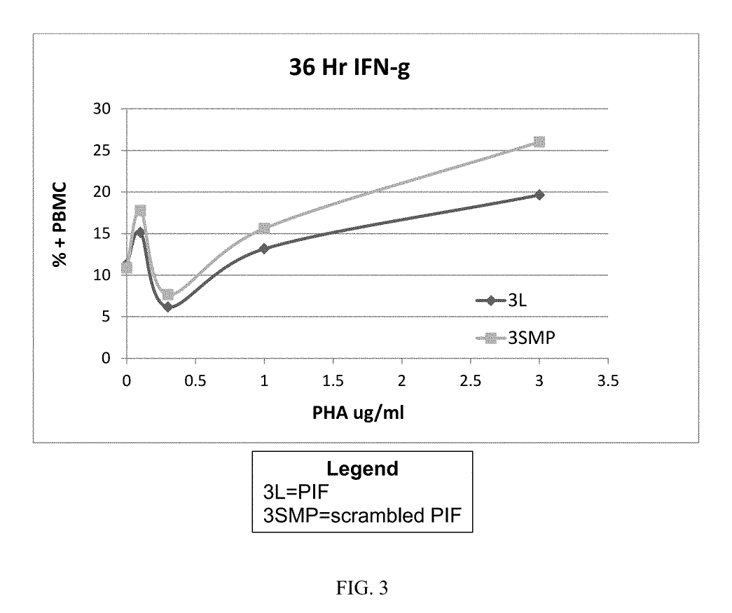

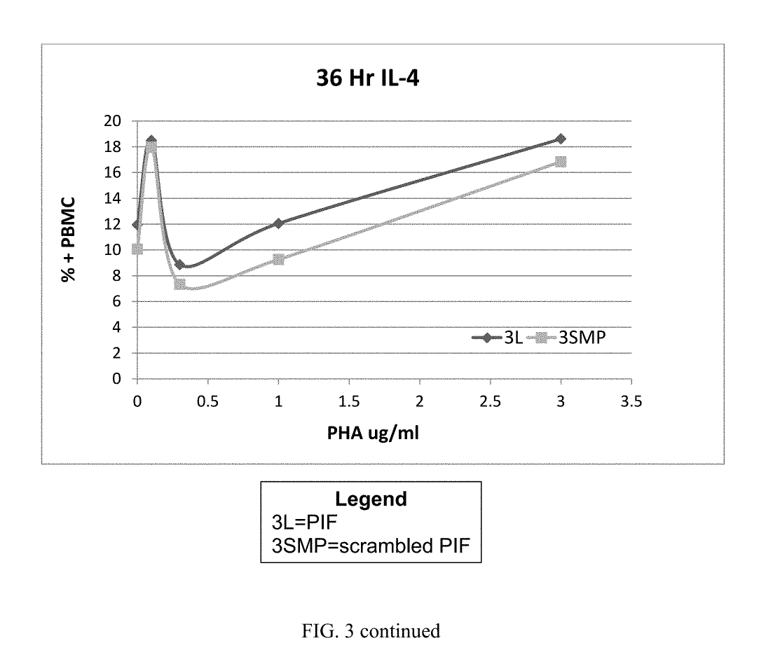

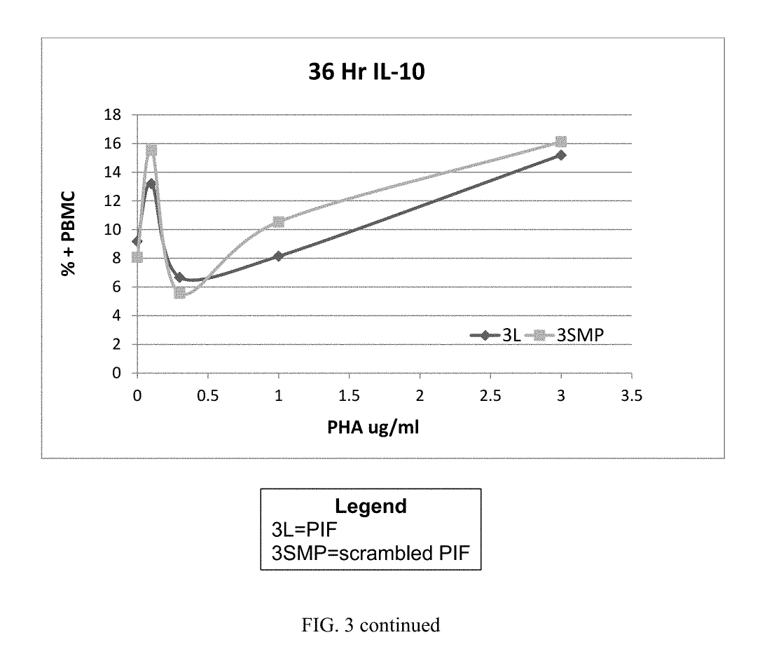

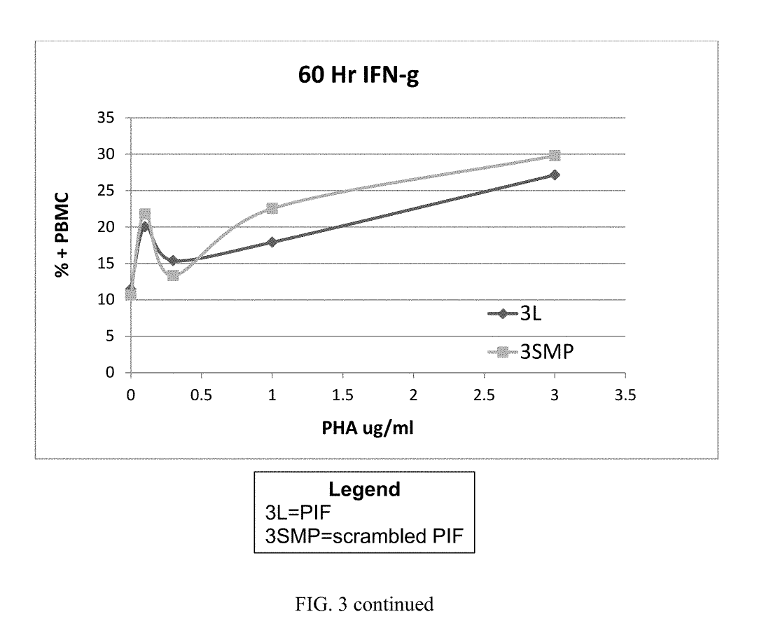

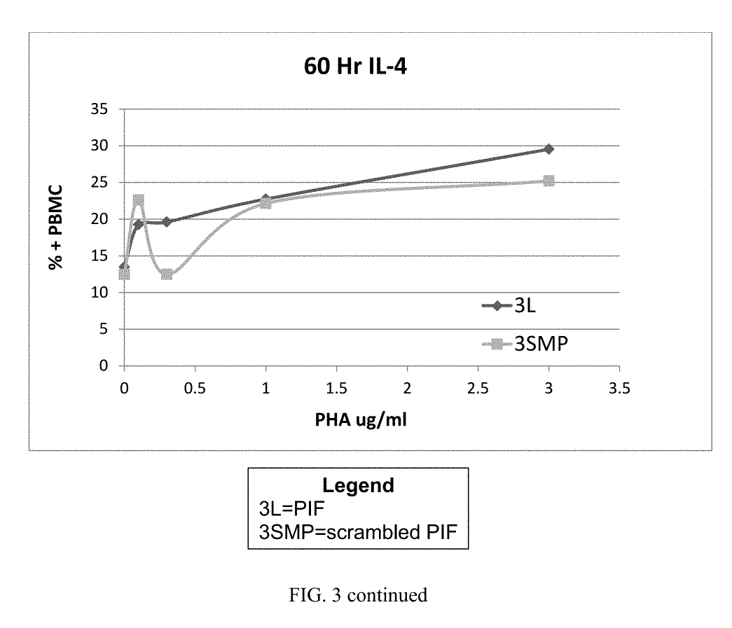

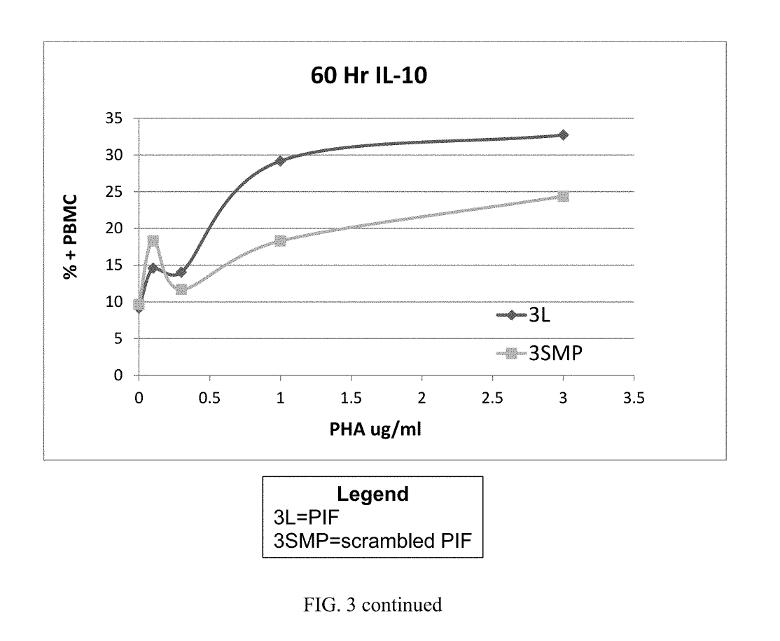

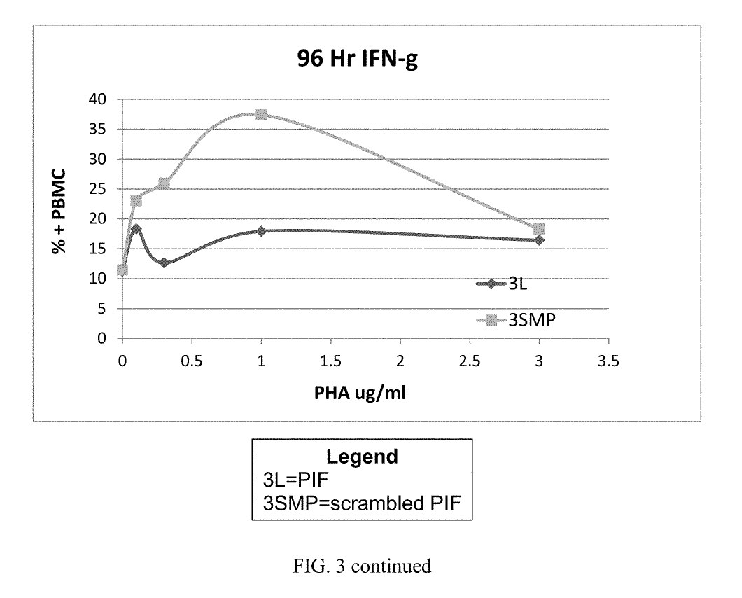

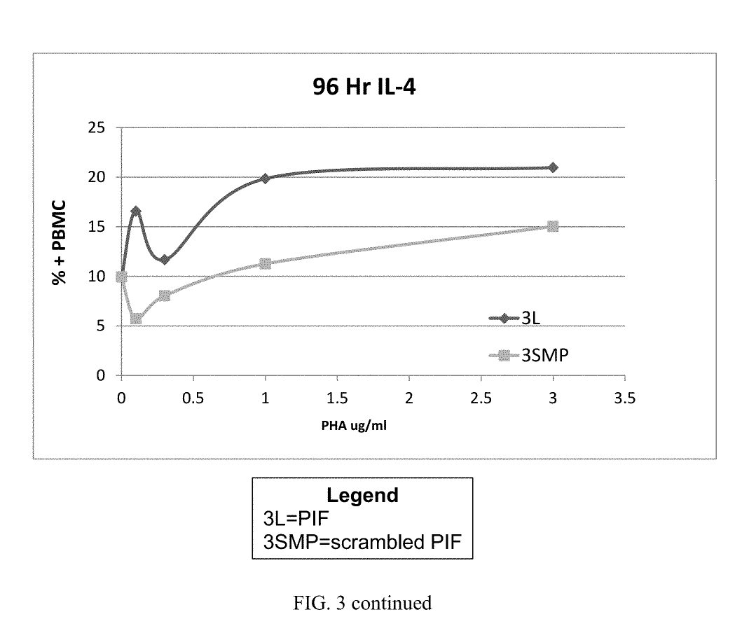

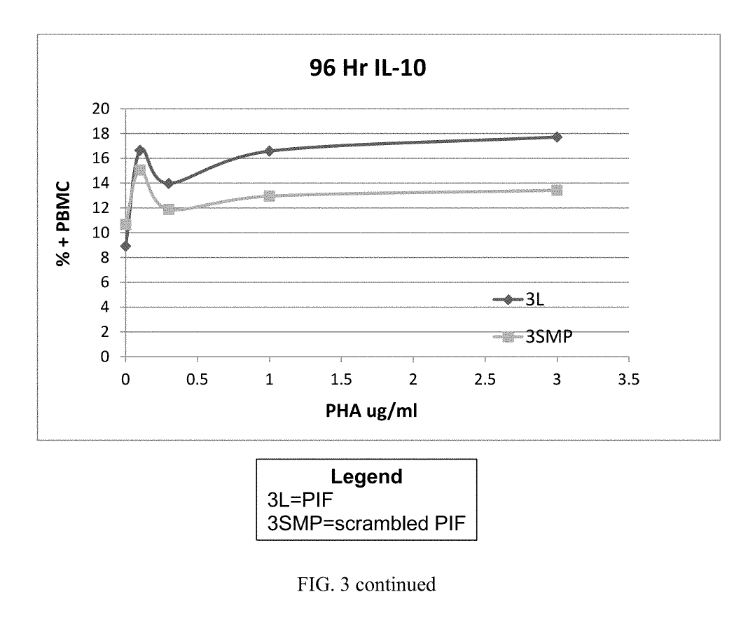

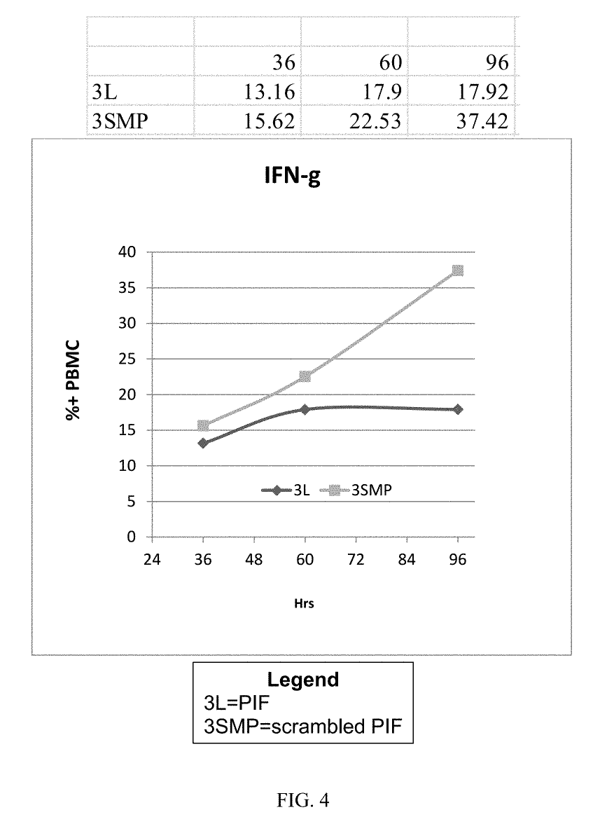

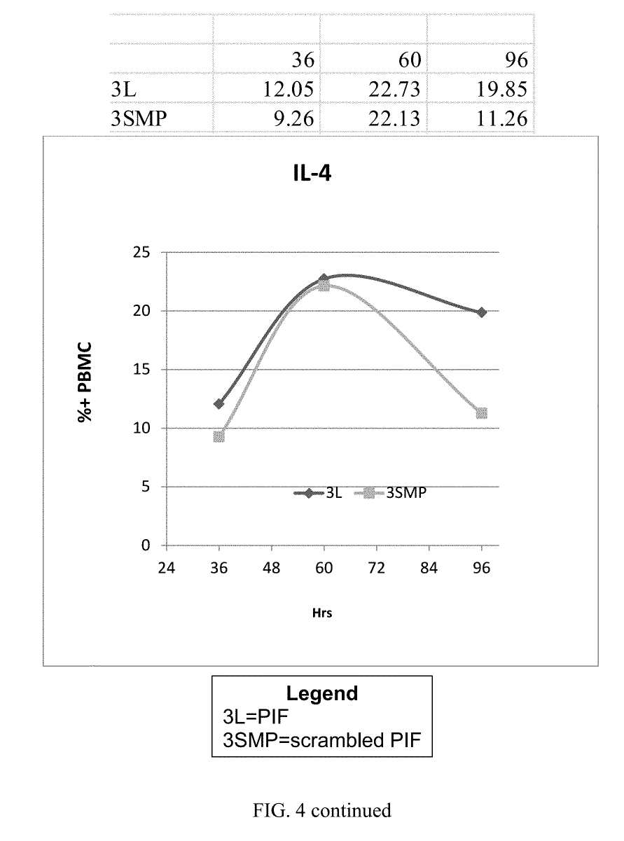

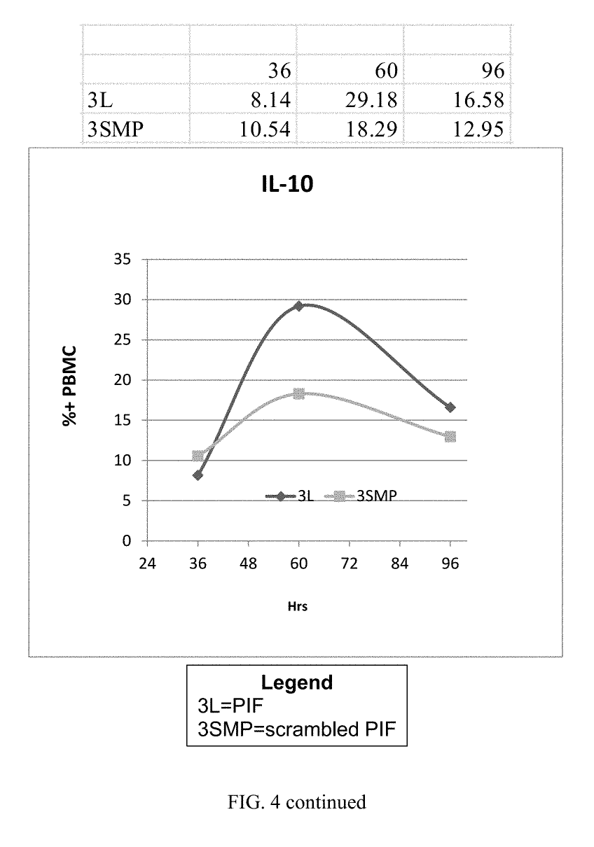

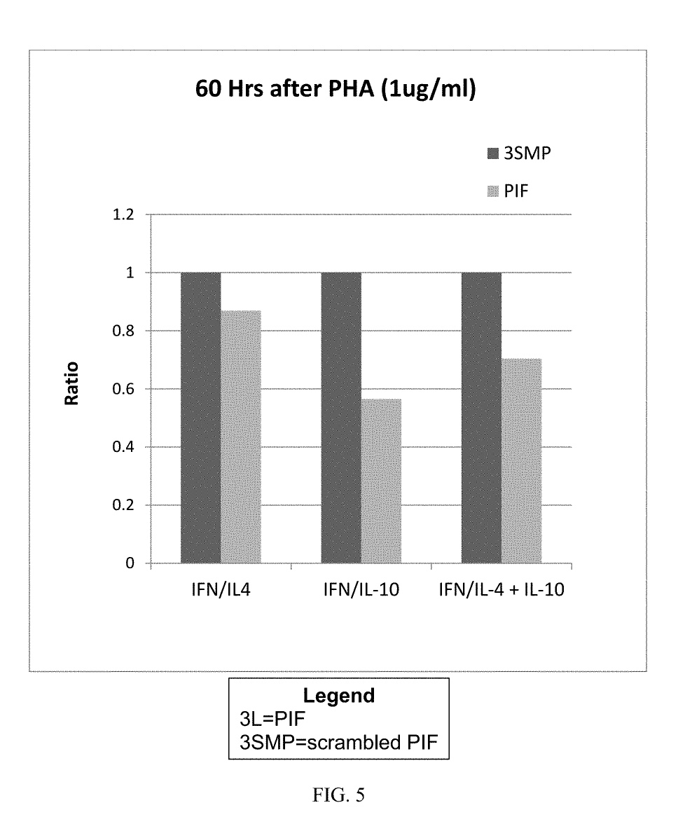

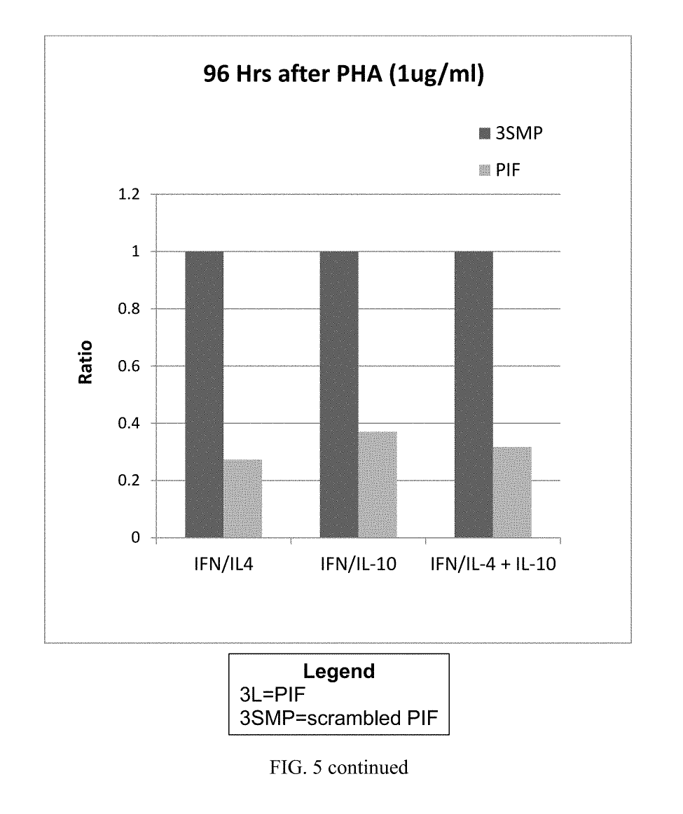

In the second experiment, the effect of PIF on the percent of the subject's lymphocytes expressing a given cytokine was determined, and the results were compared to those of the healthy control. This was carried out using PIF alone and following activation by PHA. Data shows a 24-96-hour experiment in a control subject, examining IL10, IL4, and TNFα comparing PIF to a PIFscr control. The number of IL10+ cells significantly increased compared to the control. This increase was followed by a return to baseline 96 hours after exposure to 1 μg/mL PHA. The cytokine ratio was compared to the control; 30 nM PIF led to a decrease in the pro/anti-inflammatory ratio (TNF/IL10/IL4). In addition, when the effect of 0-4 μg/mL PHA on these cytokines was examined, a dose-dependent response was noted, wherein the maximal effect of PIF compared to control was noted at 4 Lμg/mL (FIGS. 3-5, Tables 6-8).

TABLE 6
LOW-DOSE PHA ACTIVATION AND EFFECT OF
PIF ON CYTOKINE PROFILES BY PBMC

| | | IFN-γ | | | | |
|---|---|---|---|---|---|---|
| | | 0 | 0.1 | 0.3 | 1 | 3 |
| 36 Hr | 3 L | 11.31 | 15.13 | 6.16 | 13.16 | 19.64 |
| | 3 SMP | 10.85 | 17.75 | 7.66 | 15.62 | 26.02 |
| 60 Hr | 3 L | 11.45 | 20.01 | 15.38 | 17.9 | 27.14 |
| | 3 SMP | 10.66 | 21.78 | 13.33 | 22.53 | 29.78 |
| 96 Hr | 3 L | 11.21 | 18.3 | 12.63 | 17.92 | 16.42 |
| | 3 SMP | 11.46 | 23.04 | 25.95 | 37.42 | 18.3 |

TABLE 7
LOW-DOSE PHA ACTIVATION AND EFFECT OF
PIF ON CYTOKINE PROFILES BY PBMC

| | | IL-4 | | | | |
|---|---|---|---|---|---|---|
| | | 0 | 0.1 | 0.3 | 1 | 3 |
| 36 Hr | 3 L | 11.95 | 18.5 | 8.86 | 12.05 | 18.61 |
| | 3 SMP | 10.07 | 17.97 | 7.33 | 9.26 | 16.84 |
| 60 Hr | 3 L | 13.48 | 19.25 | 19.6 | 22.73 | 29.52 |
| | 3 SMP | 12.45 | 22.62 | 12.47 | 22.13 | 25.2 |
| 96 Hr | 3 L | 9.85 | 16.56 | 11.67 | 19.85 | 20.96 |
| | 3 SMP | 9.94 | 5.72 | 8.04 | 11.26 | 15.03 |

TABLE 8
LOW-DOSE PHA ACTIVATION AND EFFECT OF PIF
ON CYTOKINE PROFILES BY PBMC

| | | IL-10 | | | | |
|---|---|---|---|---|---|---|
| | | 0 | 0.1 | 0.3 | 1 | 3 |
| 36 Hr | 3 L | 9.18 | 13.19 | 6.67 | 8.14 | 15.19 |
| | 3 SMP | 8.07 | 15.53 | 5.57 | 10.54 | 16.12 |
| 60 Hr | 3 L | 9.13 | 14.57 | 14.03 | 29.18 | 32.71 |
| | 3 SMP | 9.65 | 18.27 | 11.69 | 18.29 | 24.38 |
| 96 Hr | 3 L | 8.92 | 16.64 | 13.97 | 16.58 | 17.72 |
| | 3 SMP | 10.66 | 15.06 | 11.88 | 12.95 | 13.42 |

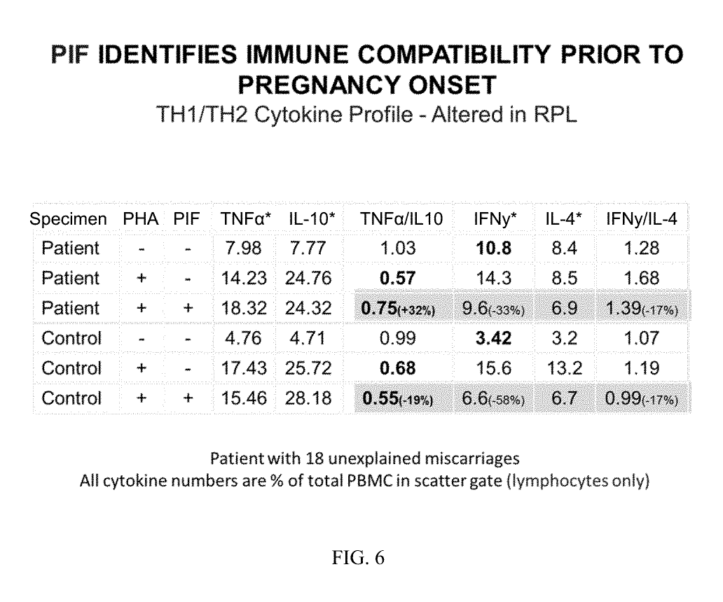

Subsequently, the RPL subject was compared to the healthy control subject. Tie data showed major changes in a number of cytokines. In the presence of PHA, the TNFα/IL10 ratio decreased in both the RPL and control subjects. In contrast, in the presence if PIF, the TNFα/IL10 ratio increased in the RPL subject, but decreased in the control subject. The INFγbasal expression was higher in the RPL subject. PHA further increased the NFNγbasal expression in the RPL subject, while in the control subject a four-fold increase was noted. However, in the presence of PIF, INFγbasal expression decreased almost three-bid in the RPL subject. In the RPL subject, the baseline IL4 was high; it was unaffected by PHA but reduced by PIF. In the control subject, the baseline IL4 was low; PHA increased it four-fold, while PIF reduced it by the same amount. The INFg/IL4 ratio behaved similarly (FIG. 6). In both basal and PHA-induced changes in cytokines, a difference in response, such as increased Th1/Th2, indicates immune dysregulation.

### EXAMPLE 3

PIF Targets 14-3-3, Heat Shock and Di-Isomerase Proteins to Regulate Immune Response: Evidence for Immune Targeting In Vivo

PIF Peptide Synthesis

Synthetic PIF (MVRIKPGSANKPSDD; 15 aa) and scrambled PIF (PIF scr; GRVDPSNKSMPKDIA) were synthesized by solid-phase peptide synthesis (Peptide Synthesizer, Applied Biosystems) employing Fmoc (9-fluorenylmethoxycarbonyl) chemistry at Bio-Synthesis, Inc. Final purification was carried out by reversed-phase HPLC and identity was verified by matrix-assisted laser desorption/ionization time-of-flight mass spectrometry and amino acid analysis at >95% purity.

In Vitro Surface Plasmon Resonance (SPR) Spectroscopy Studies

All SPR experiments were carried out using a BIAcore X unit (GE Healthcare). Experiments were performed at 37° C. at a constant flow rate of 10 μL/min using HBS-EP (10 mM HEPES and 150 raM NaCl supplemented with 3 mM EDTA and 0.005% (v/v). Surfactant P20 was adjusted to pH 7.4 as a running buffer. First, optimal immobilization conditions for PIF15, the RP (used as a negative control) and TLR4-MD2 were determined via pH scouting. Covalent immobilization of these peptides to the carboxylated dextran matrix of a CMS chip (GE Healthcare) was carried out using standard amine coupling using 10 mM sodium acetate, adjusted to pH 5.0 for PIF 15 and TLR4-MD2 or pH 4.0 for RP, as an immobilization buffer. For all experiments utilizing a PIF 15 sensor surface, the RP was immobilized to the first reference flow cell (FC1) and PIF 15 was immobilized to the ‘downstream’ flow cell (FC2). Sensorgrams are presented as the reference subtracted signal (i.e. FC2−FC1). CD14 and TLR4-MD2, suspended in HBS-PE at a concentration of 1 μM, were each passed over the PIF 15 sensor surface to assess whether the effect on PBMCS occurs via engagement of PIF with CD 14 or TLR4-MD2. Phytohemagglutinin (PHA) from p.vulgaris (Sigma-Aldrich), rough (Ra) LPS from E. coli EH100 (Sigma-Aldrich) and smooth LPS from e.coli 055:B5 (Sigma-Aldrich) were passed over PIF 15 sensor surfaces to examine whether the regulatory effects of PIF toward stimulant (PHA and LPS) activity on PBMCs was the result of a direct interaction between PIF and the stimulant or from PIF having a cognate cellular effect. Stimulants were suspended in HBS-PE (at 2.5, 5 or 10 μM for PHA or 5, 25 or 100 μM for LPS). A PIF-specific monoclonal antibody (clone PIF-1/GENH1.12.7 (Genway Technologies) was suspended in HBS-PE (at 1000, 500 or 250 nM) and loaded over the PIF sensor surface as a positive control. A TLR4 sensor surface was also generated (TLR4 was immobilized in FC2) to further assess the possibility of an interaction between PIF and TLR4. PIF was suspended in HBS-NE at a concentration of 0.5mM and loaded over the TLR4 sensor surface.

PBMC Binding Studies

Non-pregnant subjects who underwent infertility treatment signed standard informed consent. Studies were approved CARI Institute, Chicago, Ill.). Blood was drawn as part of the work-up process with the use of excess specimen without identifiers (n=12). Additional samples were obtained. PBMCs were isolated from peripheral blood (Ficoll Hypaque density gradient method). PBMCs were incubated with FITC-PIF, FITC-PIFscr, and size-matched irrelevant peptide at (0-100 uM) concentrations along with an antibody cocktail (anti-CD3, CD4,CD8, CD25, FoxP3; BD Pharmingen). Isotype antibodies were used as negative controls. Two- and three-color staining was performed. Fluorescence measurements (20,000-50,000 gated events/sample) by Coulter Epics XL Flow Cytometer were analyzed with System II software (BeckmanCoulter).

PBMCs

A whole blood unit was obtained from three different non-pregnant healthy donors after obtaining consent. Following separation by using Ficoll-hypaque, isolated PBMCs were passed through each unit separately using CD14, CD4 or CD8 affinity columns. Subsequently, the cells were washed with PBS and frozen in a serum-free media and were shipped at −80° C. to Eprogen for further processing.

CD14, CD8 and CD4 Cell Extraction

A PIF-resin affinity column was specifically designed for this study, to replace the commonly used multistep method. The data showed only the PIF column as compared with the control (an agar-only column was able to extract specific proteins). Briefly, to PIF 15, a carbon spacer (C6) at N-terminus followed by a Cysteine at the end and then the thiol group of the cysteine was conjugated to agarose resin (Biosynthesis, TX). The protocol for extractions of cells was as follows: 50 μL/mL of PIF resin was centrifuged for 1 min (6,000×g) and washed twice with 150 μL of a non-detergent lysing buffer (NDLB) (Eprogen) in a compact reaction tube (CRT) (Becton-Dickenson) by centrifugation. A vial containing 8-10M cells was lysed with 1.5 mL of NDLB by two freeze-thaw cycles to −80° C. and the resulting lysate centrifuged at ˜6000×g. 450 μL of the Lysate supernatant were added to the CRT containing washed resin, and it was incubated for 1 hr at 4° C. with intermittent vortexing to ensure good PIF resin-protein contact. The tubes were centrifuged for 1 min (6000−g) and then washed twice with 100 mL NDLB. Filtrates were combined and diluted to 400 μL total volume for ProteoSep® RP HPLC runs. The Lysate-treated resin was extracted twice with 150 μL of 0.1 M Glycine-HCl solution by vortexing for 10 min and then centrifuged for 1 min. The resulting filtrates containing the PIF extracted proteins were combined and frozen at −80° C. prior to MS analysis.

Proteomic MS Analysis: Trypsin Digestion

In-solution trypsin digestion of the protein extracts was conducted using the Filter-Assisted Sample Preparation digestion kit (FASP) according to the manufacturer's procedure (Protein Discovery, Expedeon). Briefly, 40 μL of protein lysate extract from above was reduced with 4 μmol DTT at room temperature for 1 h. The sample was mixed with 200 μL of urea sample solution in the spin filter and centrifuged at 14,000×g for 15 min Sample flow-through was discarded after washing with another 200 μL of urea sample solution. Proteins on the spin filter were alkylated with iodoacetamide in 90 μL urea sample solution for 20 min in the dark. The proteins in the filter were washed twice with 100 μl urea sample solution and centrifuged at 14,000×g for 10 min. Then, 100(0.1 of 50 mM ammonium bicarbonate (NH4HC03) were added to the spin filter and centrifuged at 14,000×g for 10 min and repeated two more times. Trypsin digestion was conducted at 37° C. overnight using a trypsin:protein ratio of 1:100. After incubation, the spin filter was washed twice with 40 μl of 50 mM NH4HC03 and centrifuged at 14,000×g for 10 min, collecting the filtrate into a clean tube. Peptides were extracted by adding 50 μl of 0.5M sodium chloride solution and centrifuging at 14,000×g for 10 min. The collected filtrate containing the tryptic peptides was acidified with 5 μl formic acid and desalted via CI8 solid phase extraction (SPE) (Supelco Discovery SPE, Sigma Aldrich). The filtrate was dried under vacuum and tryptic peptides were resuspended in 20 μL 0.1% formic acid for subsequent LC-MS/MS analysis.

Proteomic MS Analysis: LC-MS/MS Analysis

The samples were analyzed by reversed phase nanoflow liquid chromatrography and tandem mass spectrometry (LC-MS/MS) using an Easy nLC-II system (Thermo) coupled to a Thermo LTQ Velos dual pressure linear ion trap system (Thermo). Standard equine cytochrome C digest was injected as a quality control. Two microliters of sample were loaded via the autosampler onto a trap column (EASY-Column 2 cm, ID 100 μm, 5 μm, C18-A) then directed to an analytical column (EASY-Column, 10 cm, ID 75 um, 3 um, C18-A2) at a flow rate of 250 nL/min. The mobile phase consisted of solvent A (99.9% water with 0.1% formic acid) and solvent B (99.9% acetonitrile with 0.1% formic acid). Separation was achieved using a run time of 100 min. The first linear gradient was from 2% to 40% B over 90 min, and the second linear gradient was from 40% to 80% B over 5 min and held for 5 min before returning to the initial mobile phase composition (2% B). Tandem mass spectra (MS/MS) were acquired on the top 10 most abundant ions at a given chromatographic time point by data-dependent scanning.

Proteomic MS Analysis: Peptide/Protein Identification

All tandem mass spectra were extracted by Xcalibur version 2.7.0 and analyzed by Sequest (Proteome Discoverer, Thermo) and X! Tandem. Sequest (v. 1.3.0.339) and X! Tandem were set up to search a trypsin-indexed reversed concatenated IPI mouse protein database (v3.86, 119068 entries) with a fragment ion mass tolerance of 0.8 Da and a parent ion tolerance of 2.0 Da. Carbamidomethylation of cysteine was specified in Sequest and X! Tandem as a fixed modification and oxidation of methionine was as variable modification. Scaffold 3 (ProteomeSoftware, Portland, Oreg.) was used to compile Sequest search results and validate MS/MS based peptide and protein identifications. Peptide identifications were accepted if they could be established at greater than 95.0% probability as specified by the Peptide Prophet algorithm. Protein identifications were accepted if they could be established at greater than 99.9% probability and contained at least 2 identified peptides. Protein probabilities were assigned by the Protein Prophet algorithm. Label-free relative abundance quantitation was done by a spectral counting approach.

PIF-Binding Studies

C57BL/6 mice were injected intravenously or intraperitoneally with 100 μL of 500 nM FITC-PIF. After 5 or 30 min, respectively, mice were sacrificed, immersed in a hexane dry-ice bath, embedded in frozen media, and 40 um whole-body sections were made. Sections were dehydrated and scanned using a Typhoon™ 9140 bioanalyzer (GE Healthcare) set at an excitation wavelength to image FITC-PIF fluorescence (295 nm). White blood cells or splenocytes were collected from C57BL/6 mice exposed to FITC-PIF for 1 hr on ice. Cells were washed and re-suspended in 1 mL of FACS buffer (Becton-Dickinson) and the percentage of cells binding FITC-PIF was measured using two-color flow-cytometer with gating to PE/CD45+ labeled cells (Becton-Dickinson).

Flow Cytometry Studies

White blood cells or splenocytes were collected from C57BL/6 mice exposed to FITC-PIF at different concentrations for 1 hr on ice. Cells were washed and resuspended in 1 mL of FACS buffer (Becton-Dickinson) and percentage of cells binding FITC-PIF was measured. Identification of the cell type associated with PIF's binding to circulating murine immune cells was tested. Immune cells were collected following sacrifice. The collected cells were incubated with solutions of FITC-PIF, 12.5-50 μg/mL, along with anti-CD45 (BD Pharmingen). Isotype controls served as negative controls. Two-color staining was done using conventional techniques. Fluorescence measurements (20,000-50,000 gated events per sample) were performed in a Coulter® Epics® XL™ Flow Cytometer using System II software for data acquisition and analysis (Beckman Coulter, Inc.).

Statistical Analysis

Data were analyzed by one-way analysis of variance (ANOVA) with Dunnett error protection and a confidence interval of 95% was calculated using Analyse-it® for Microsoft Excel (Analyse-it Software, Ltd.) for data analysis. Values of P0.05 were considered statistically significant. Pathway analysis was performed using the Ingenuity Systems software, ranking by greatest number of genes in a given pathway Protein probabilities were analyzed using Protein Prophet algorithm software. Protein targets clustering and interaction was determined using String 9.1 version software.

PIF Acts Directly on PBMCs

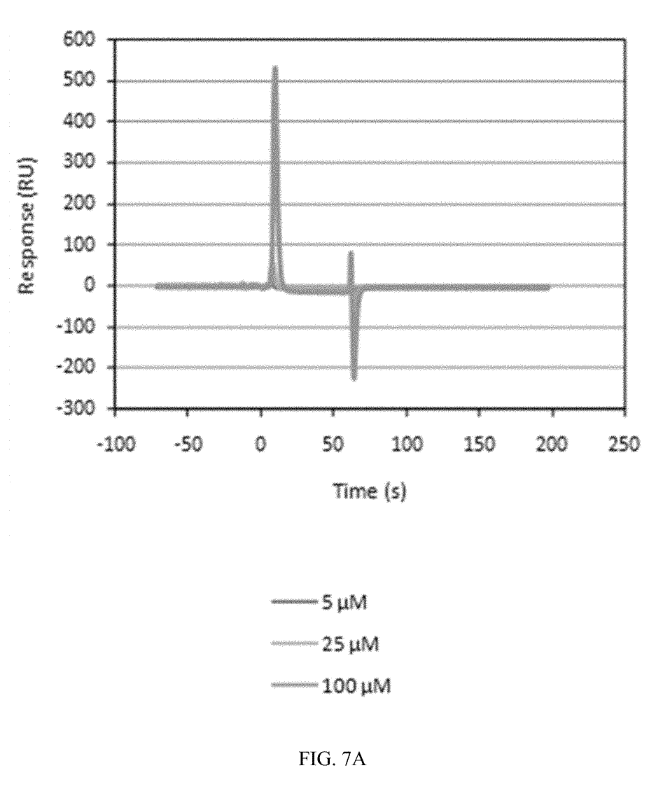

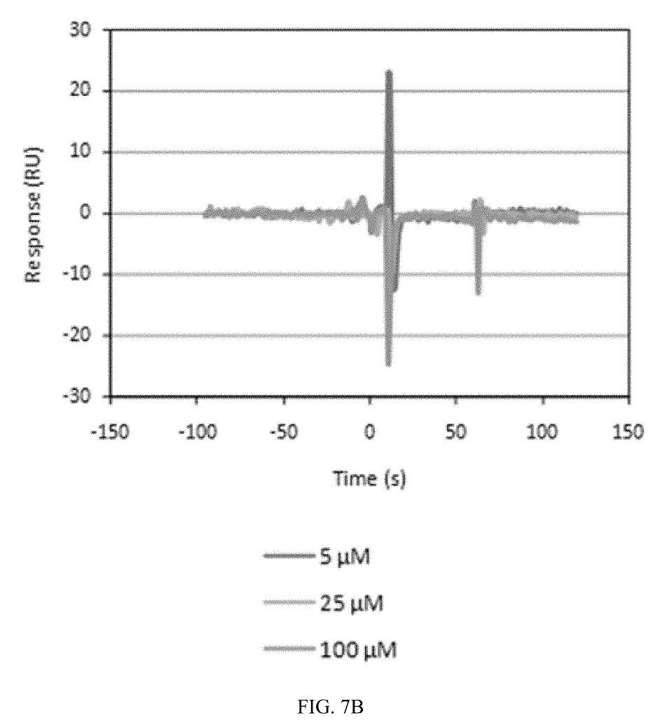

PIF prevents LPS (lipopolysaccharide, a bacterial antigen)-induced nitric oxide (NO) production by macrophages). Therefore, it was important to determine whether PIF acts directly on immune cells, or whether the inhibitory action is a inhibitory effect due to direct peptide-LPS interaction. The interaction potential between PIF and rough (Ra LPS) or smooth (O55:B5 LPS) LPS was assessed via a robust and sensitive surface plasmon resonance (SPR) method. This method utilizes a laser beam which deviates if ligand-sensor interaction takes place. The use of anti-PIF monoclonal antibody confirmed that PIF attachment to the sensor surface is active. Subsequently, the two LPS molecules at 5, 25 and 1OO uM concentration were passed over the PIF attached sensor (FIGS. 7A and 7B). The data demonstrated no observable LPS (ligand) and PIF-sensor interaction at all concentrations tested. LPS mainly acts by targeting the CD 14 receptor on macrophages to activate the immune synapse. However, LPS also can act independently of the CD 14 receptor. In addition, PIF primarily targets unstimulated CD 14+ cells.

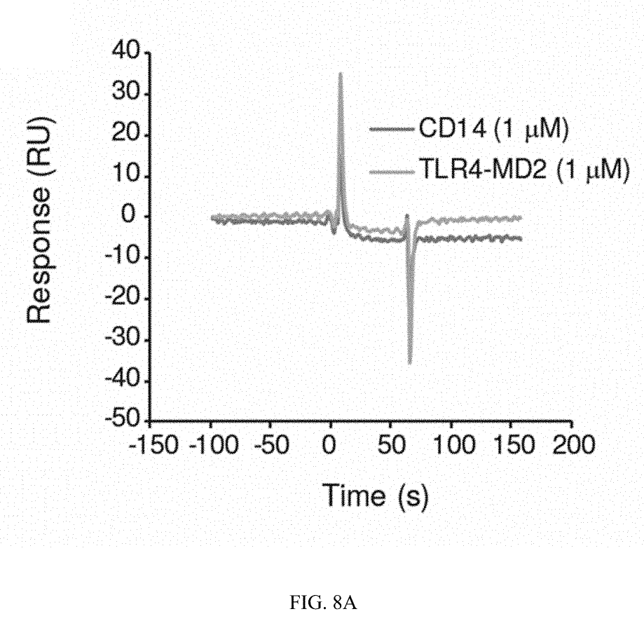

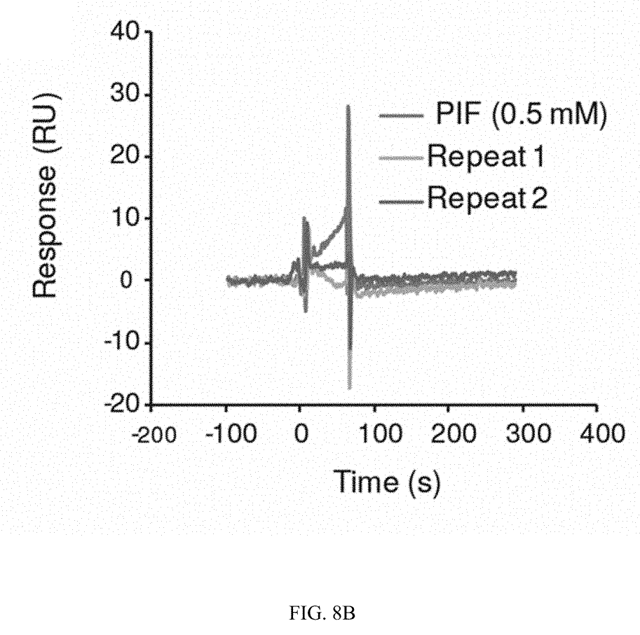

Therefore, we have examined whether PIF interacts directly with immune cells via the CD 14 receptor or whether it alternatively targets the TLR4-MD2 site downstream. Inhibition of the TLR-4 pathway blocks PIF's effect on immune cells. However, this information does not totally exclude whether the ligand itself is targeted by PIF. The SPR-based analysis has showed that PIF targets neither the receptor itself nor its downstream mediator TLR4-MD2, even when tested at high concentrations (FIG. 8A). To further confirm this lack of interaction, TLR4-MD2 surfaces were also constructed and exposed to a high concentration (0.5 mM) of PIF (FIG. 8B). Even at such a high concentration, no appreciable binding of PIF to the immobilized TLR4-MD2 could be observed. Thus, PIF acts through cognate cellular process, involving specific targets, rather than through secondary interaction via interaction with activating agents.

PIF Tarets CD4+/CD25+/Fox P3+Cells

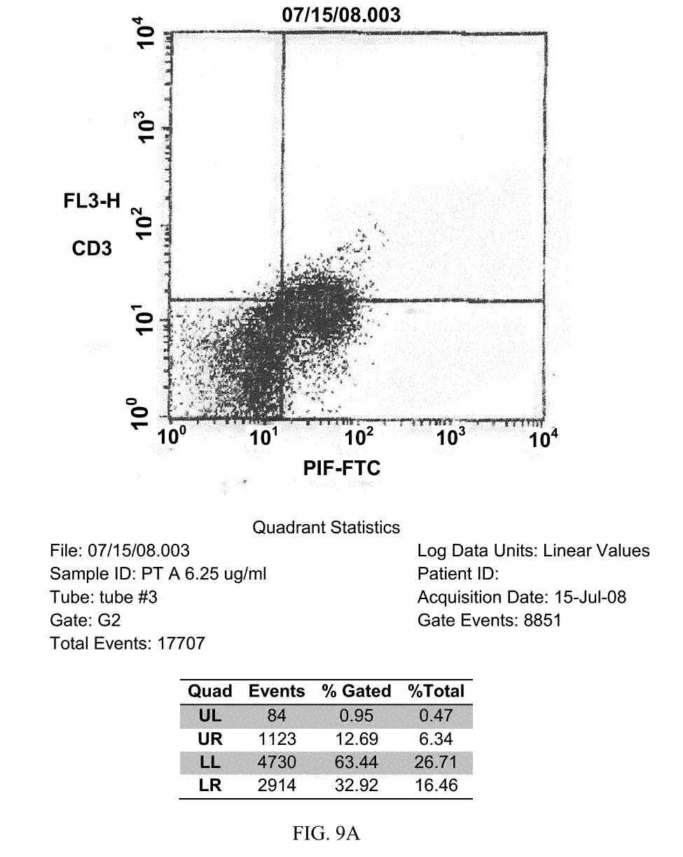

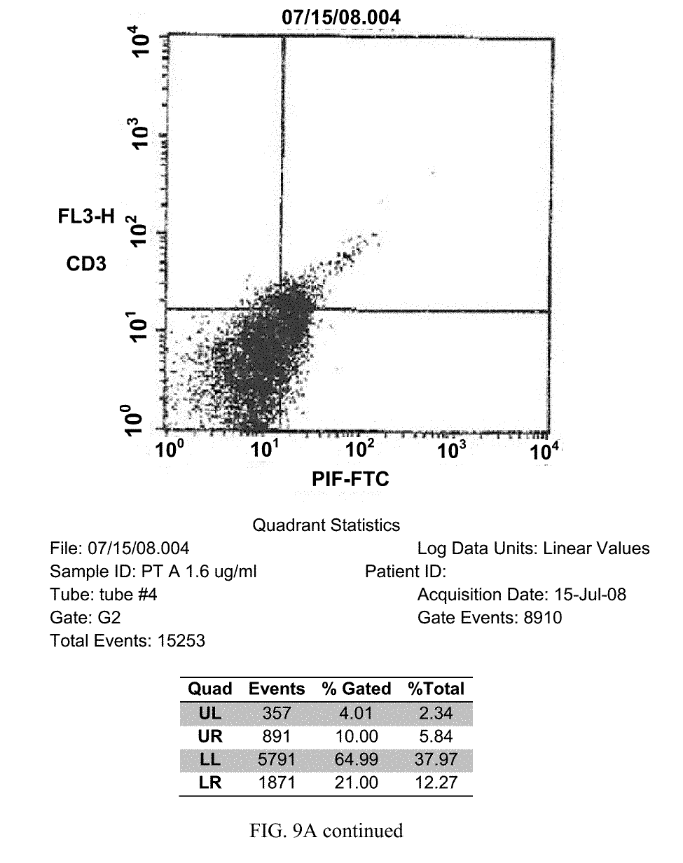

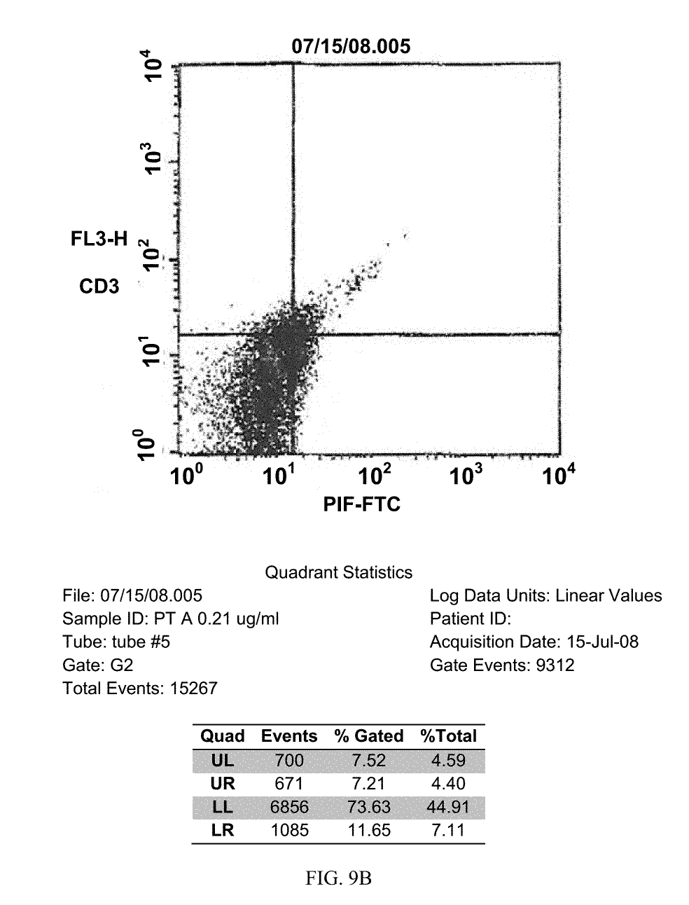

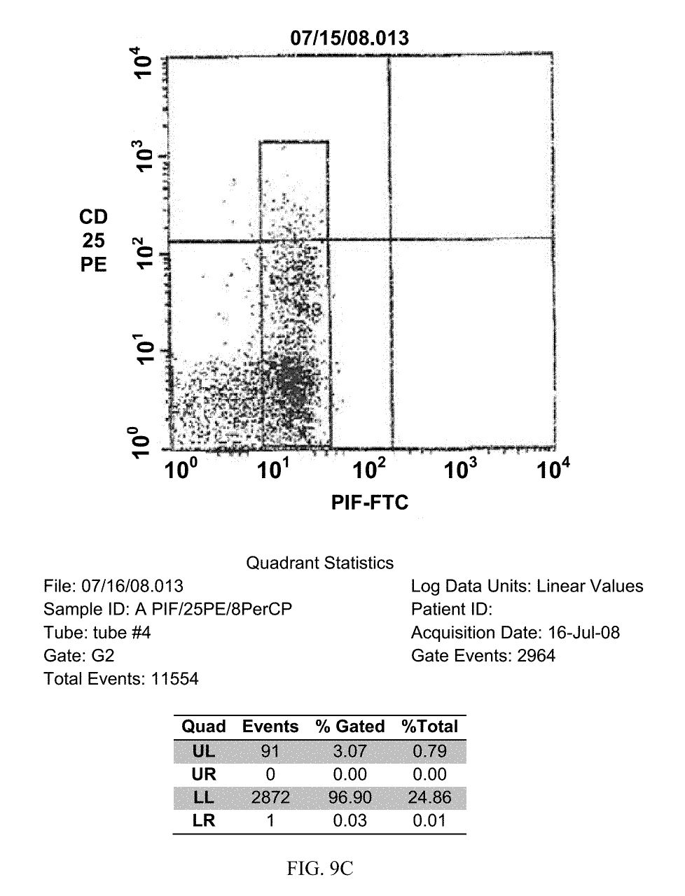

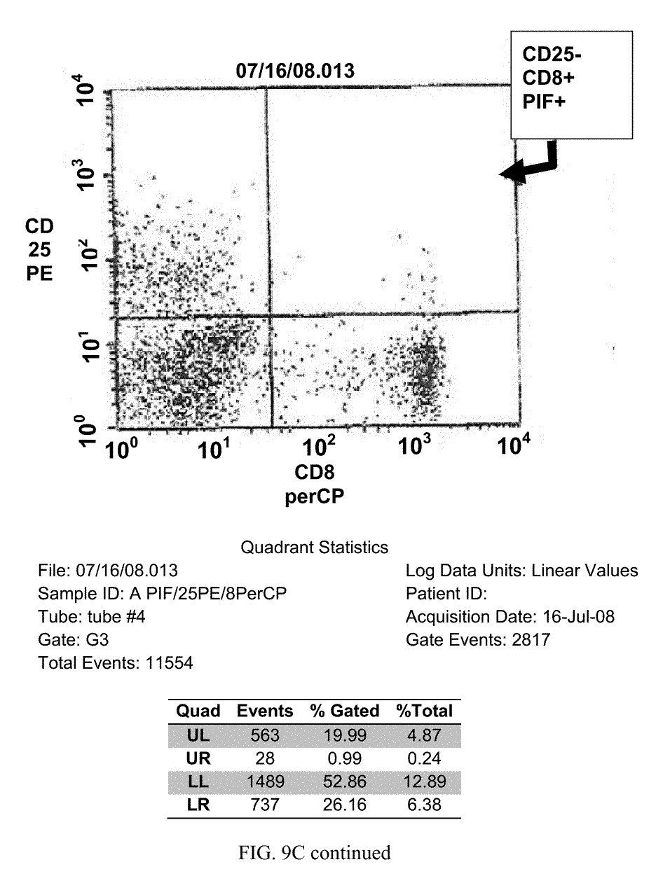

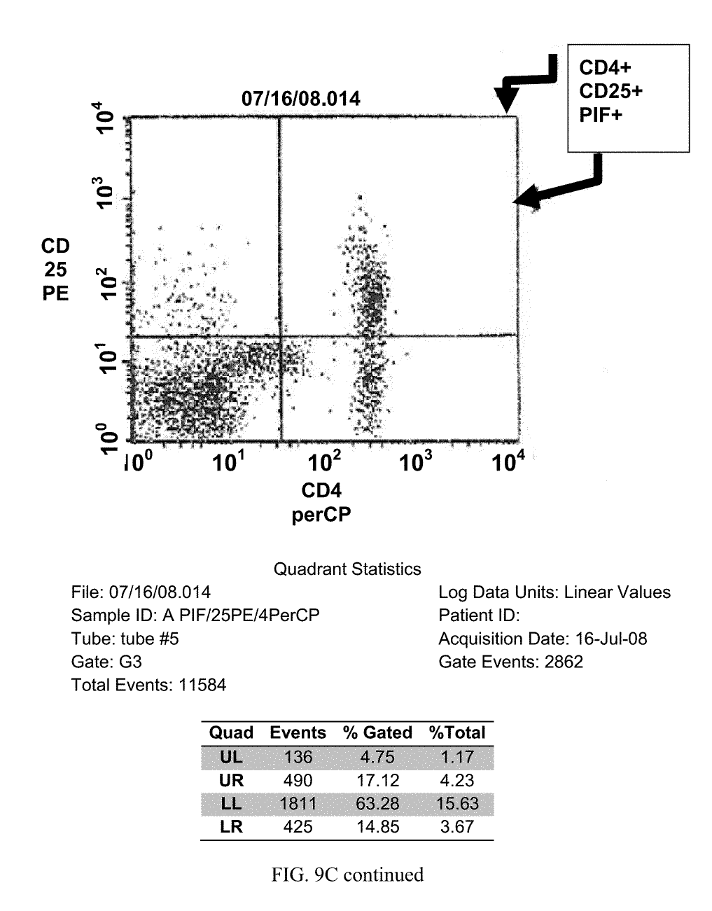

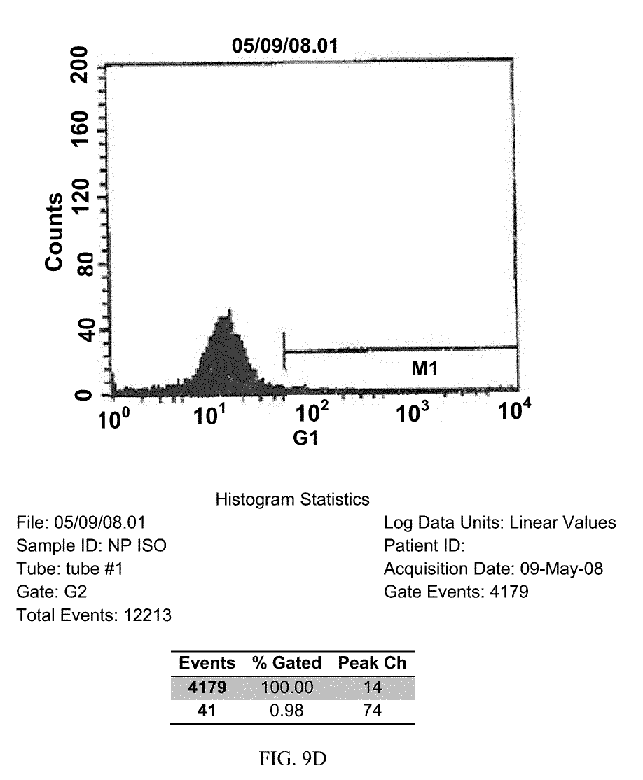

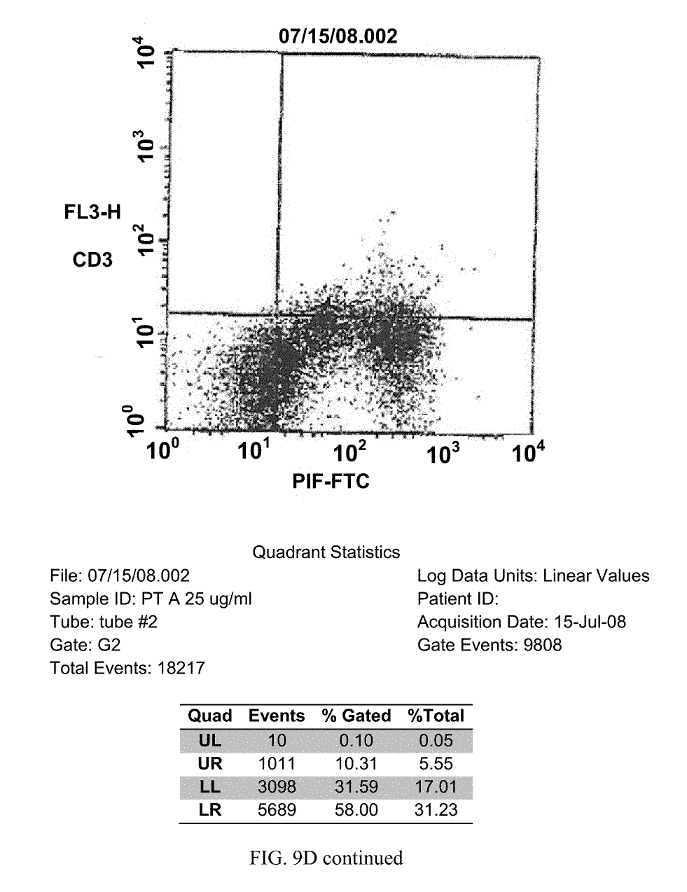

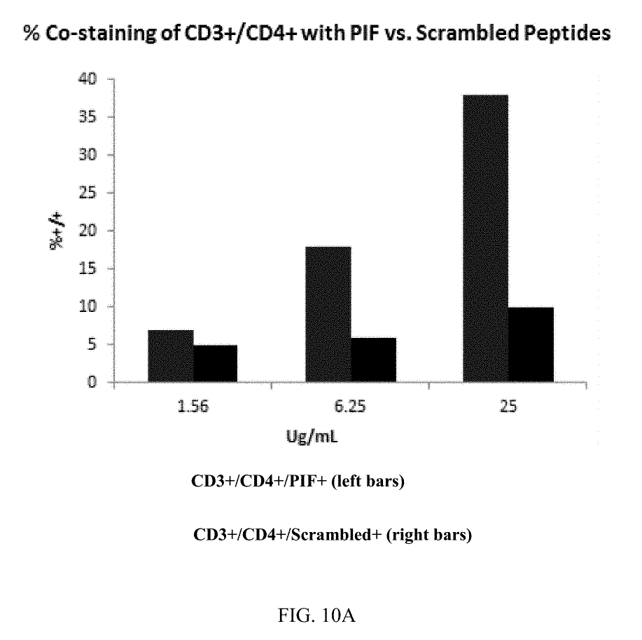

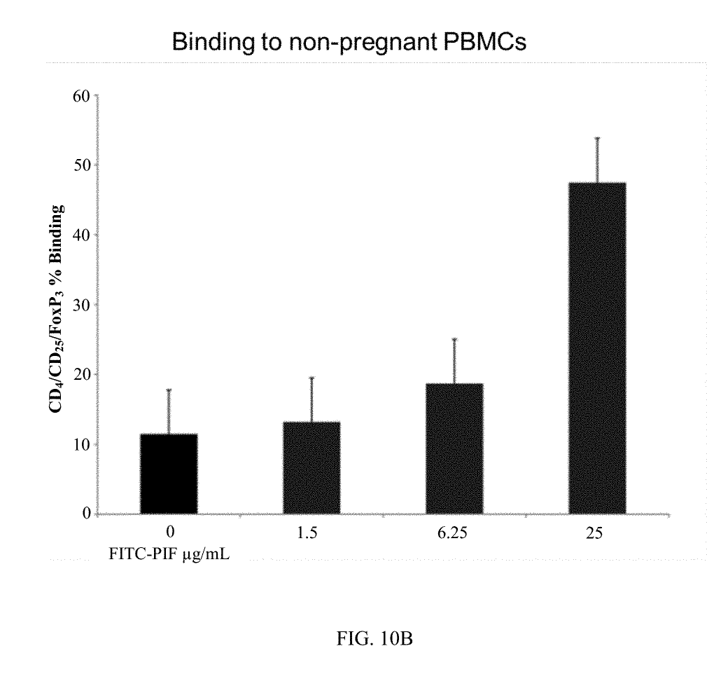

PIF binds <10% of T cells, an effect which is greatly magnified in the presence of a mitogen. Since regulatory T-cells play a major role in tolerance, we have further examined whether PIF interacts with this important cell subtype in naive cells (FIG. 9). Using two-color flow cytometry, we examined FITC-PIF binding to naive CD3+ cells, showing dose-dependent binding (FIGS. 9A and 9B). Further binding to CD4+/CD25+ cells was determined, showing that PIF targets these Treg cells (FIG. 9C). In contrast, PIF failed to bind to gated CD8+/CD25+ cells, reflecting the specificity of its interaction (data not shown). FIG. 9D shows that the isotype control showed no significant binding, indicating PIF's specificity. Subsequently, we examined whether PIF targeted cells are CD4+/CD25+/FoxP3+. FIGS. 10A and 10B show that FITC-PIF binding to these cells is dose-dependent and the binding is amplified in high peptide doses, as compared to scrambled PIF, which is known to have minimal binding. Such data indicates that PIF specifically binds regulatory T-cells.

PIF Targets Proteins in Unstimulated Human CD14, CD4, and CD8 Cells

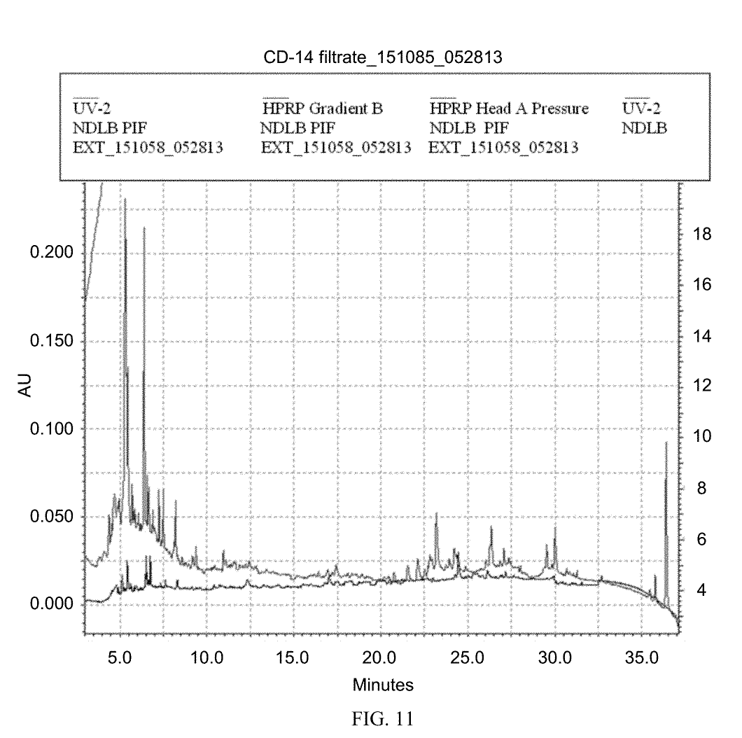

A novel method for identification of PIF proteins targets in the embryo has been developed and validated. The method is based on PIF affinity chromatography followed by mass spectrometry. PIF targets unstimulated CD 14+ cells; however, it does not bind to the receptor or its downstream mediator. Therefore, we aimed to identify specific PIF targets in immune cells in both innate (CD14+) cells and those belonging to the adaptive arm of immunity (CD4 and CD8 cells). Using one whole unit of blood from a healthy donor, PBMCs were first separated and subsequently passed through an anti-CD 14+ antibody column reaching >95% purity. The collected cells were then extracted and passed through an anti-PIF based affinity chromatography. FIG. 11 shows the chromatography profile of CD14+ cells following extraction. A very large number of proteins were identified in the extract. In contrast, following extraction in the filtrate the number of proteins was low, indicating an intimate PIF-protein interaction. Clearly, only a small portion of the total proteins present are retained on the PIF affinity resin, while most of the proteins in the lysate are eluted. Subsequent re-extraction of the remaining un-retained proteins after the first extraction showed that no additional proteins were extracted from the lysate, indicating that the PIF affinity column extraction was complete and specific to the proteins identified by MS. It is important to note that the use of non-detergent lysing buffers is critical. When lysing buffers were used containing detergents no successful protein extraction was observed.

Similarly, CD4+ and CD8+ PBMCs were isolated from a whole unit of blood followed by purification using an anti-CD4+ and anti-CD8+ antibody columns, respectively. The collected cells were each lysed and extracted in the same manner as the CD14+ cells, by using semi-quantitative mass spectrometry. The collected cells were each extracted and the proteins collected were identified. When the main cellular location of the PIF protein targets was examined ,the majority were found at a cytoplasmic location; others were present in the nucleus, and rarely, in the membrane. Such observation indicates that the novel chromatography method is a robust means for identifying PIF binding partners. The proteins isolated from these PIF affinity column extractions were then analyzed using LC/MS/MS. Tables 9-11 below show that >70 protein targets in CD14 cells were identified by PIF affinity extraction and LCMSMS analysis, several of which were iso-proteins belonging to the same class. Tables 12 and 13 detail the proteins identified for the CD4 and CD8 cell lysates clearly showing that PIF extracts to be very similar for these lymphocytes. Furthermore, when comparing the CD 14 proteins identified to those for the CD4 and CD8 cells (Table 14), a clear picture emerges showing PIF specifically targets well defined classes of proteins in these immune cell lineages and the proteins extracted for all three lineages are nearly identical. This specificity in targeting should help in deciphering the nature of PIF regulation of the immune response.

TABLE 9

| | |
|---|---|
| CD14 | |
| 14-3-3 protein eta | |
| 14-3-3 protein gamma | |
| 14-3-3 protein theta | |
| 14-3-3 protein zeta/delta | x |
| 60S ribosomal protein L22 | |
| HSPA5 (70) | |
| 78 kDa glucose-regulated protein | |
| Acidic leucine-rich nuclear phosphoprotein 32 family | x |
| member A | |
| Actin, cytoplasmic 2 | |
| Actinin alpha 1 isoform 3 | |
| Activated RNA polymerase 11 transcriptional | |
| coactivator pl5 | |
| Annexin Al | |
| Apolipoprotein B receptor | |
| Bridging integrator 2 | |
| Calmodulin (Fragment) | x |
| Calreticulin | x |
| CD14 | |
| Centrosomal protein of 120 kDa (Fragment) | |
| Coronin-IA | |
| Elongation factor 1-beta (Fragment) | |
| Endoplasmin | |
| Filamin-A | |
| Gelsolin | |
| Glucosidase 2 subunit beta | xx |
| Glyceraldehyde-3-phosphate dehydrogenase | |
| Heat shock protein HSP 90-beta O | xx- |
| Hematopoietic lineage cell-specific protein | |
| Hepatoma-derived growth factor | x |
| Histone H4 | |
| Histone-binding protein RBBP7 | |
| Hornerin | |
| Isoform 1 of Vinculin | |
| Isoform 2 of Adenylyl cyclase-associated protein 1 | |
| Isoform 2 of Heat shock protein HSP 90-alpha | xx |
| Isoform 2 of Leucine-rich repeat flightless- | |
| interacting protein 1 | |
| Isoform 2 of Nucleophosmin | |
| Isoform 2 of Protein SET | x |
| Isoform 2 of Ras suppressor protein 1 | |
| Isoform H14 of Myeloperoxidase | |
| Isoform Short of 14-3-3 protein beta/alpha | |
| Isoform SV of 14-3-3 protein epsilon | |
| TGFBP1 | |
| Latent-transforming growth factor beta-binding | |
| protein 1 | |
| Lymphocyte-specific protein 1 | |
| Matrin-3 | |
| Myeloid cell nuclear differentiation antigen | |
| Myosin-9 | |
| Nuclear autoantigenic sperm protein | |
| CD14 | |
| Nuclear ubiquitous casein and cyclin-dependent | |
| kinase substrate 1 | |
| Nuclease-sensitive element-binding protein 1 | |
| (Fragment) | |
| Nucleolin | x |
| Plastin-2 | |
| Platelet factor 4 variant | |
| Protein disulfide-isomerase | |
| Protein disulfide-isomerase A4 | |
| Protein S100-A8 | |
| Ras GTPase-activating-like protein IQGAP1 | |
| Serine/arginine-rich-splicing factor 1 | |
| Serine/arginine-rich-splicing factor 2 (Fragment) | |
| Serum deprivation-response protein | x |
| Talin-1 | |
| Thrombospondi n-1 | x |
| Thymosin alpha-1 | x |
| Thyroid hormone receptor-associated protein 3 | |
| Transgelin-2 | |
| Transitional endoplasmic reticulum ATPase | |
| Tropomodulin-3 | |
| Tropomyosin alpha-1 chain | |
| Tropomyosin alpha-3 chain | |
| Tropomyosin alpha-4 chain | x |
| Tubulin alpha-4A chain | |
| Tumor protein D52-like 2, isoform CRA e | |
| Vimentin | |

TABLE 10
CD4

| 14-3-3 protein zeta/delta | |
|---|---|
| Acidic leucine-rich nuclear phosphoprotein 32 family member A | |
| Actin, cytoplasmic 1 | |
| Calmodulin | |
| Calreticulin | PDIA2 partner |
| Cartilage oligomeric matrix protein | Thrombospondin 5 |

Cofilin-1
Hepatoma-derived growth factor
Isoform 2 of Heat shock protein HSP 90-alpha
Isoform 2 of Protein SET
Nuclease-sensitive element-binding protein 1 (Fragment)
Nucleolin
Serine/arginine-rich-splicing factor 1
Serum albumin
Serum deprivation-response protein
Talin-1
Thrombospondin-1
Thrombospondin-4 chain
Tubulin alpha-lC chain

TABLE 11
CD8

14-3-3 protein zeta/delta
Acidic leucine-rich nuclear phosphoprotein 32 family member A
Actin, cytoplasmic 1
Calmodulin
Calreticulin
Cartilage oligomeric matrix protein Cofilin-1
Glucosidase 2 subunit beta
Hepatoma-derived growth factor
Histone H2A type 1-B/E
Isoform 11 of Titin
Isoform 2 of Heat shock protein HSP 90-alpha
Isoform 2 of Protein SET
Nucleolin
Serine/arginine-rich-splicing factor 1
Serine/arginine-rich-splicing factor 2
Serum albumin
Serum deprivation-response protein
Thrombin light chain
Thrombospondin-1
Thrombospondin-4
Thymosin alpha-1
Tropomyosin alpha-4 chain
Tubulin alpha-lC chain

TABLE 12
CD8

| | | Protein | | | | |
|---|---|---|---|---|---|---|
| | | Molecular | Inject | Inject | Inject | |
| Protein name | Protein accession numbers | Weight | 1 | 2 | 3 | Ave |
| Thrombospondin-1 | TSP1_HUMAN | 129,381.70 | 7 | 7 | 7 | 7 |
| Serum albumin | sp \| P02768 \| ALBU_HUMAN | 69,366.90 | 3 | 6 | 8 | 6 |
| Cartilage oligomeric | B40KJ3_HUMAN, COMP_HUMAN, | 77,211.80 | 4 | 4 | 5 | 4 |
| matrix protein | G3XAP6HUMAN | | | | | |
| Thombospondin-4 | E7E519_HUMAN, TSP4_HUMAN | 96,005.30 | 3 | 3 | 3 | 3 |
| Actin, cytoplasmic 1 | ACTB_HUMAN | 41,737.80 | 3 | 3 | 3 | 3 |
| Tropomyosin alpha-4 | sp\|P67936\|TPM4_HUMAN | 28,522.40 | 3 | 3 | 3 | 3 |
| chain | | | | | | |
| Thymosin alpha-1 | B8ZZQ6_HUMAN, sp\|P06454- | 12,073.40 | 2 | 2 | 4 | 3 |
| | 2\|PTMA_HUMAN | | | | | |
| Isoform 2 of Heat | sp\|P07900-2\|HS90A_HUMAN, | 84,663.20 | 2 | 2 | 2 | 2 |
| shock protein HSP 90- | sp\|P07900\|HS90A_HUMAN | | | | | |
| alpha | | | | | | |
| Calreticulin | CALR_HUMAN | 48,142.90 | 2 | 2 | 2 | 2 |
| Serum deprivation- | SDPR_HUMAN | 47,172.90 | 2 | 2 | 2 | 2 |
| response protein | | | | | | |
| Acidic leucine-rich | AN32A_HUMAN | 28,586.10 | 2 | 2 | 2 | 2 |
| nuclear phosphoprotein | | | | | | |
| 32 family member | | | | | | |
| 14-3-3 protein | 1433Z_HUMAN, E7EX29_HUMAN | 28,037.30 | 2 | 2 | 2 | 2 |
| zeta/delta | | | | | | |
| Calmodulin | CALM_HUMAN, E7ETZ0_HUMAN, | 16,838.00 | 2 | 2 | 2 | 2 |
| | H0Y7A7_HUMAN | | | | | |
| Talin-1 | TLNl_HUMAN | 269,765.10 | 1 | 1 | 1 | 1 |
| Nucleolin | NUCL HUMAN | 76,615.90 | 1 | 1 | 1 | 1 |
| Tubulin alpha-1C | F5H5D3_HUMAN, G3V1U9_HUMAN, | 50,135.70 | 1 | 1 | 1 | 1 |
| chain | TBA1A_HUMAN; TBA1B_HUMAN | | | | | |
| Nuclease-sensitive | HOY449_HUMAN, YBOX1_HUMAN | 35,923.80 | 1 | 1 | 1 | 1 |
| element-binding | | | | | | |
| protein 1 (Fragment) | | | | | | |
| Isoform 2 of Protein | sp\|Q01105-2\|SET_HUMAN | 32,103.30 | 1 | 1 | 1 | 1 |
| SET | | | | | | |
| Serine/arginine-rich- | J3KII2_HUMAN, sp\|Q07955- | 28,329.70 | 1 | 1 | 1 | 1 |
| splicing factor 1 | 2\|SRSFl_HUMAN, sp\|Q07955-3\|SRS | | | | | |
| Hepatoma-derived | A8K8G0_HUMAN, Q5SZ07_HUMAN, | 22,964.00 | 1 | 1 | 1 | 1 |
| growth factor | sp\|P51858-2\|HDGF_HUMAN, | | | | | |
| Cofilin-1 | COF1_HUMAN, E9PP50_HUMAN | 18,503.20 | 1 | 1 | 1 | 1 |

TABLE 13
CD4

| | | Protein | | | | |
|---|---|---|---|---|---|---|
| | | Molecular | Inject | Inject | Inject | |
| Protein name | Protein accession numbers | Weight | 1 | 2 | 3 | Ave |
| Cartilage oligomeric | B4DKJ3_HUMAN, COMP_HUMAN, | 79,694.20 | 10 | 10 | 11 | 10 |
| matrix protein | G3XAP6_HUMAN | | | | | |
| Serum albumin | sp\|P02768\|ALBU_HUMAN | 69,366.90 | 4 | 7 | 12 | 8 |
| Thromboipondin-1 | TSP1_HUMAN | 129,381.70 | 6 | 6 | 6 | 6 |
| Thymosin alpha-1 | B8ZZQ6_HUMAN, sp\|P06454- | 12,073.40 | 4 | 5 | 7 | 5 |
| | 2\|PTMA_HUMAN | | | | | |
| Isoform 2 of Heat 1 | sp\|P07900-2\|H590A_HUMAN, | 84,663.20 | 4 | 4 | 4 | 4 |
| shock protein HSP | sp\|P07900\|HS90A_HUMAN | | | | | |
| 90-alpha | | | | | | |
| Calreticulin | CALR_HUMAN | 48,142.90 | 4 | 4 | 4 | 4 |
| Tropomyosin alpha- | sp\|P67936\|TPM4_HUMAN | 28,522.40 | 4 | 4 | 4 | 4 |
| 4 chain | | | | | | |
| Isoform 2 of Protein | sp\|Q01105-2\|SET_HUMAN | 32,103.30 | 3 | 4 | 4 | 4 |
| SET | | | | | | |
| Thombospondin-4 | E7E519_HUMAN, TSP4_HUMAN | 96,005.30 | 3 | 3 | 3 | 3 |
| Thrombin light chain | E9PIT3_HUMAN, THRB_HUMA N | 65,408.20 | 3 | 3 | 3 | 3 |
| 14-3-3 protein | 1433Z_HUMAN, E7EX29_HUMAN | 27,745.90 | 3 | 3 | 3 | 3 |
| zeta/delta | | | | | | |
| Hepatoma-derived | A8K8G0_HUMAN, Q5SZ07_HUMAN, | 26,788.60 | 3 | 3 | 3 | 3 |
| growth factor | spP51858-2\|HDGF_HUMAN, | | | | | |
| Calmodulin | CALM_HUMAN, E7ETZ0_HUMAN, | 16,838.00 | 3 | 3 | 3 | 3 |
| | H0Y7A7_HUMAN | | | | | |
| Tubulin alpha-lC | F5H5D3_HUMAN, G3V1U9_HUMAN, | 50,135.70 | 2 | 2 | 3 | 2 |
| chain | TBA1A_HUMAN, BA1B_HUMAN | | | | | |
| Nucleolin | NUCL_HUMAN | 76,615.90 | 2 | 2 | 2 | 2 |
| Serum deprivation- | SDPR_HUMAN | 47,172.90 | 2 | 2 | 2 | 2 |
| response protein | | | | | | |
| Actin, cytoplasmic 1 | ACTB_HUMAN | 41,737.80 | 2 | 2 | 2 | 2 |
| Serine/arginine-rich- | J3KII2_HUMAN, sp\|Q07955- | 28,329.70 | 2 | 2 | 2 | 2 |
| splicing factor 1 | 2\|SRSF1_HUMAN, sp\|Q07955-3\|SRS | | | | | |
| Serine/arginine-rich- | B4LN89_HUMANSRSF2_HUMAN | 25,477.10 | 1 | 2 | 3 | 2 |
| splicing factor 2 | | | | | | |
| Histone H2A type 1- | H2A1B_HUMAN, H2A1C_HUMAN, | 14,136.10 | 2 | 2 | 2 | 2 |
| B/E | H2A1D_HUMAN | | | | | |
| Isoform 11 of Titin | sp\|Q8WZ42-11\|TITIN_HUMAN-R, | 0 | 2 | 2 | 2 | 2 |
| | sp\|Q8WZ42-1 | | | | | |
| Glucosidase 2 | K7ELL7_HUMAN, sp\|P14314- | 59,425,80 | 1 | i | 1 | i |
| subunit beta | 2\|GLU2B HUMAN, | | | | | |
| Acidic leucine-rich | AN32A_HUMAN | 28,586.10 | 1 | 1 | 1 | 1 |
| nuclear | | | | | | |
| phosphoprotein 32 | | | | | | |
| family member | | | | | | |
| Cofilin-1 | COF1_HUMAN, E9PP50 HUMAN | 18,503.20 | 1 | 1 | 1 | 1 |

TABLE 14
PIF targets in CD14+ cells

| | | Molecular | Inject | Inject | Inject | |
|---|---|---|---|---|---|---|
| Identified Proteins | Accession Number | Weight | 1 | 2 | 3 | Ave |
| Oxidative Stress and Protein Misfolding | | | | | | |
| Transitional | TERA_HUMAN | 89 k Da | 3 | 2 | 3 | 3 |
| endoplasmic reticulum | | | | | | |
| ATPase | | | | | | |
| Protein disulfide- | PDIAl_HUMAN | 57 kDa | 3 | 2 | 2 | 2 |
| isomerase | | | | | | |
| Protein disulfide- | PD1A4_HUMAN | 73 kDa | 4 | 1 | 2 | 2 |
| isomerase A4 | | | | | | |
| Calreticulin | CALR_HUMAN | 48 kDa | 7 | 4 | 6 | 6 |
| Nuclear autoantigenic | sp \| P49321 \| NASP_HUMAN | 85 kDa | 2 | 0 | 1 | 1 |
| sperm protein | | | | | | |
| 78 kDa glucose- | GRP78_HUMAN | 72 kDa | 6 | 3 | 3 | 4 |
| regulated protein | | | | | | |
| (HSP70AS) | | | | | | |
| Endoplasmin (HSP90bl) | ENPL_HUMAN | 92 kDa | 0 | 2 | 1 | 1 |
| Heat shock protein HSP | HS908_HUMAN | 83 kDa | 2 | 1 | 1 | 1 |
| 90-beta 0 | | | | | | |
| Heat shock protein HSP | sp \| P07900-2 \| | 98 kDa | 1 | 2 | 1 | 1 |
| 90-alpha-12 | HS90A_HUMAN | | | | | |
| Thromoospondin-1 | TSP1_HUMAN | 129 kDa | 7 | 5 | 8 | 7 |
| Cell survival and DNA damage control | | | | | | |
| Vimentin | BOY1C4_HUMAN (+1) | 50 kDa | 9 | 11 | 9 | 10 |
| 14-3-3 protein zeta/delta | 1433Z_HUMAN (+1) | 28 kDa | 4 | 3 | 4 | 4 |
| 14-3-3 protein eta | 1433F_HUMAN | 28 kDa | 1 | 1 | 2 | 1 |
| 14-3-3 protein theta | 1433T_HUMAN | 28 kDa | 1 | 1 | 2 | 1 |
| 14-3-3 protein gamma | 1433G_HUMAN | 28 kDa | 3 | 3 | 3 | 3 |
| 14-3-3 protein | sp\|P31946-2\| 1433B_HUMAN | 28 kDa | 3 | 3 | 4 | 3 |
| beta/alpha-1 | | | | | | |
| 14-3-3 protein epsilon-1 | sp \| P62258-2 \| | 27 kDa | 4 | 4 | 2 | 3 |
| | 1433E_HUMAN | | | | | |
| Hornerin | HORN_HUMAN | 282 kDa | 1 | 0 | 2 | 1 |
| Annexin A1 | ANXA1_HUMAN (+1) | 39 kDa | 0 | 0 | 2 | 1 |
| Macrophage and Neutrophil Activation | | | | | | |
| Myosin-9 | sp \| P35579 \| MYH9_HUMAN | 227 kDa | 11 | 9 | 10 | 10 |
| Thymosin alpha-1 | B8ZZQ6_HUMAN (+1) | 12 kDa | 5 | 5 | 4 | 5 |
| Lymphocyte-specific | sp\|P33241 \| LSP1_HUMAN | 37 kDa | 4 | 2 | 1 | 2 |
| protein 1 | | | | | | |
| Myeloperoxidase H14-1 | sp \| P05164-2 \| | 74 kDa | 2 | 2 | 2 | 2 |
| | PERM_HUMAN | | | | | |
| Myeloid cell nuclear | MNDA_HUMAN | 46 kDa | 2 | 2 | 2 | 2 |
| differentiation antigen | | | | | | |
| Calmodulin (Fragment) | H0Y7A7_HUMAN (+2) | 21 kDa | 6 | 4 | 3 | 4 |
| Histone H4 | H4 HUMAN | 11 kDa | 3 | 0 | 2 | 2 |
| Histone-binding protein | E9PC52_HUMAN | 47 kDa | 2 | 0 | 1 | 1 |
| RBBP7 | (+8) | | | | | |
| Protein S100-A8 | S10A8_HUMAN | 11 kDa | 2 | 1 | 2 | 2 |
| Cytoskeleton | | | | | | |
| Tropomyosin alpha | sp \| P67936 \| TPM4_HUMAN | 29 kD | 8 | 7 | 8 | 8 |
| | 4chain | | | | | |
| Actin, cytoplasmic 2 | ACTG_HUMAN | 42 kDa | 6 | 7 | 7 | 7 |
| Talin-1 | QSTCU6_HUMAN (+1) | 258 kDa | 5 | 6 | 8 | 6 |
| Filamin-A | QSHY54_HUMAN (+2) | 277 kDa | 5 | 3 | 7 | 5 |
| Actinin alpha 1 isoform | B7TY16_HUMAN (+3) | 107 kDa | 5 | 4 | 3 | 4 |
| 3 | | | | | | |
| Tropomyosin alpha-3 | QSVU59_HUMAN (+1) | 27 kDa | 4 | 2 | 5 | 4 |
| chain | | | | | | |
| Isoform 2of Adenylyl | sp \| Q01518-2 \| | 52 kDa | 4 | 3 | 2 | 3 |
| cyclase associated | CAP1_HUMAN | | | | | |
| protein | | | | | | |
| Isoform 1 of Vinculin | sp \| PI8206-2 \| | 117 kDa | 3 | 2 | 3 | 3 |
| | VINC_HUMAN | | | | | |
| Tubulin alpha 4A chain | A8MUBl_HUMAN (+2) | 48 kDa | 2 | 3 | 1 | 2 |
| Coronin-1 A | COR1A_HUMAN | 51 kDa | 2 | 2 | 1 | 2 |
| Gelsolin | F5H1A8_HUMAN (+4) | 81 kDa | 3 | 1 | 1 | 2 |
| Tropomyosin alpha 1 | B7Z596_HUMAN (+2) | 32 kDa | 1 | 0 | 4 | 2 |
| chain | | | | | | |
| Matrin-3 | A8MXP9_HUMAN (+4) | 100 kDa | 1 | 2 | 1 | 1 |
| Plastin-2 | B4DUA0_HUMAN (+1) | 22 kDa | 1 | 1 | 2 | 1 |
| Tropomodulin-3 | TMOD3_HUMAN | 40 kDa | 1 | 2 | 1 | 1 |
| Bridging integrator 2 | F5H0W4_HUMAN (+2) | 59 kDa | 1 | 2 | 0 | 1 |
| Isoform 2 of Ras | sp \| Q15404-2 \| | 26 kDa | 0 | 1 | 2 | 1 |
| suppressor protein 1 | RSU1_HUMAN | | | | | |
| Centrosomal protein of | D6REX9_HUMAN (+2) | 96 kDa | 2 | 0 | 0 | 1 |
| 120 kDa (Fragment) | | | | | | |

String Software Analysis: PIF Targets Four Major Protein Groups 179 in CD14 Cells: PDI/HSPs, Vimentin/14-3-3, Macrophage/Neutrophils Activation, Cell Migration and Membrane Architecture

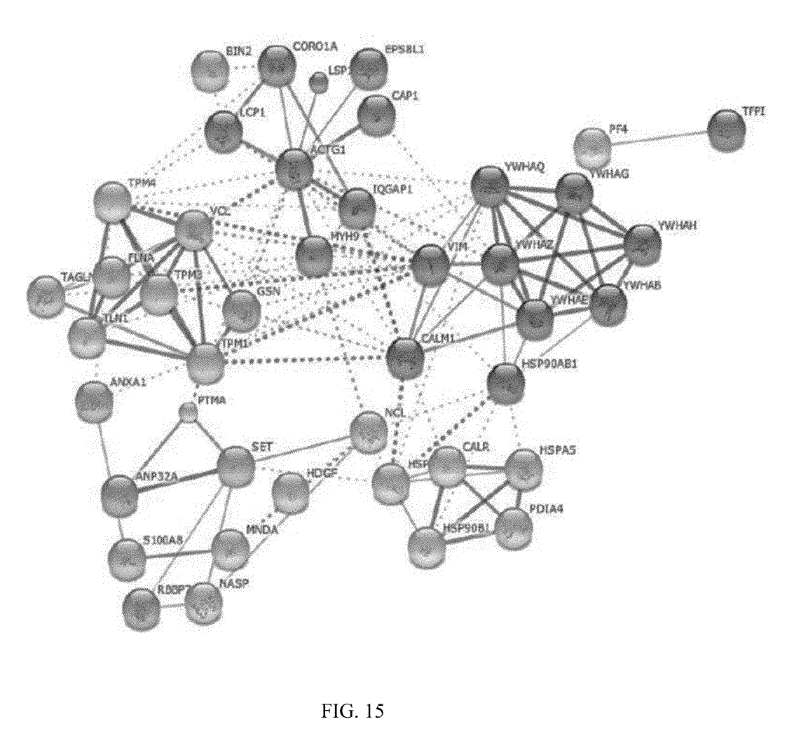

In CD 14 cells several are iso-proteins belonging to the same class. Therefore, a cluster analysis was carried out to better define the protein target groups and identify pivotal proteins which link the different groups of proteins observed (FIG. 15). The leading interactors were vimentin, calmodulin, SET-nuclear oncogene (apoptosis inhibitor) and Myosin 9 (MYH9). This analysis identified four major groups of proteins; PDI/HSPs, vimentin/14-3-3, immune activation, and those involved in the cytoskeleton. String software based analysis enabled us to determine PIF protein targets significant ranking based on Biological function: (Table 15) actin and nitric oxide regulation ranked highest coupled with most proteins also being identified in extracellular exosome as well and could be proteins that can be transported outside the cell enabling effective cell to cell communication.

TABLE 15
Cluster analysis and ranking in CD14+, CD4+ and CD8+ cells

| | Statistical Analysis |
|---|---|
| CD14 | |
| Biological Function | |
| actin binding | (1.6e−7) |
| cytoskeletal protein/RNA binding | (1.4e−4) |
| nitric-oxide synthase regulator activity | (5.9e−4) |
| The molecular function: | |
| actin binding | (1.6e−7) |
| platelet degranulation | (1.9e−7) |
| protein insertion to mitochondrial membrane | (1.9e−7) |
| Location | |
| extracellular vesicular exosome | 1.8-17e (N = 36) |
| cytosol | 4e−12 (N = 31) |
| membrane bound vesicle | (1.8e−l) |
| CD8 | |
| Biological Function | |
| response to unfolded protein | (1.8e−5) |
| response to endoplasmic reticulum stress | (9.7e−5) |
| Platelet degranulation/activation | 1.4e−2 |
| The molecular function: | |
| Integrin binding | 7.6-3 |
| Integrin binding | 7.6-3 |
| protein complex binding poly(A) RNA binding | 1.3-2 |
| Location | |
| extracellular vesicular exosome | (15 prot) 1.3e−7 |
| membrane bound vesicles | (13 prot) .3e−4 |
| CD4 | |
| Biological Function | |
| response to unfolded protein | 8.3e−6 |
| platelet degranulation/activation | 4.4e−4 |
| exocytosis | l.le−4 |
| response to endoplasmic reticulum stress | (1.9e−4) |
| The molecular function: | |
| integrin binding and protein complex binding | (4.9e−3) |
| poly(A) RNA binding | (l.le−2) |
| nitric oxide synthase regulator | (l.l-e−2) |
| Location | |
| extracellular vesicular exosome | (2e−9) |
| membrane bound vesicle | (6.4e−6) |
| organelle lumen | (1.2e−4) |

In CD14+ Cells, PIF Targets Vimentin and PDI: Role in Oxidative Stress

Following extraction, the CD14+ proteins were identified using mass spectrometry. As listed in Tables 9-11 above, approximately 70 protein targets were identified. PIF both regulates cytokine secretion and expression, as well as several other genes in unstimulated or activated human PBMCs. The 14-3-3 group is the highest represented protein group ˜10% of all targets identified. Their structure is highly similar functioning as dimers associating two different subtypes. These multifunctional scaffold phospho-serine/phospho-threonine binding proteins are involved in cell signaling, responding to stress and blocking pro-apoptotic signals, Bad and Bax. They target several proteins, enzymes and peptides as well. Thus, 14-3-3 proteins could control DNA damage. 38 213 The highest ranking of this group was 14-3-3 z/d which regulates platelets, mast cells activation and apoptosis. This protein targets CDC25A/B/C cell division cycle promoters which increased (6 fold) in co-activated PBMCs (Geo______). The iso-14-3-3 epsilon protein regulates viral replication and apoptosis. 14-3-3 gamma binds to insulin-like growth factor receptor involved in glucose metabolism. 38 217 The 14-3-3theta is involved neural degeneration. Overall PIF's targeting and possibly regulating 14-3-3 proteins gives credence to PIF's role in a large and diverse gamut of cellular functions. Therefore, we examined whether PIF binds to these targets and whether PIF can also regulate their expression. Vimentin was the highest-ranking protein which PIF targets. In macrophages, this protein regulates oxidative stress and plays a major role in response to sepsis. Vimentin expression decreased (2.2-fold) at 4 h following PBMC co-activation. Further targets were protein-di isomerase/thoredoxin (PDI), which reduce cellular stress dysfunction. PIF targets two proteins, PDI and PDI A4, which are major proteins of this group. The PDI molecule contains four thioredoxin domains. The RIKP active site of PIF targets PDI and HSPs.

PIF Targets and Regulates Heat Shock and 14-3-3 Proteins: Role in Regulating Protein Misfolding and Cell Survival

Proper protein folding and cellular protection are critical for cellular function. PIF targets the HSP cluster of proteins: HSP 90B-O, HSP 90B1, Iso2-HSPA, and HSP70A5 that controls this important process. Only in co-activated PBMCs were the HSP 90/B genes upregulated while HSPs 70 involved in stress response expression were reduced.

The highest number of PIF binding targets identified within a group were the 14-3-3 proteins, which were ˜10% of all identified targets. These are multifunctional scaffold phospho-serine/phospho-threonine binding proteins that play an important role in cell signaling, response to stress signaling, and blocking pro-apoptotic signals Bad and Bax. They target several proteins, enzymes and peptides as well. The highest ranking among them was 14-3-3 z/d. The 14-3-3 z/d protein regulates platelets, mast cell activation, and apoptosis. Thus, 14-3-3 could be involved in controlling DNA damage. PIF increased (2.8-fold) expression in naive PBMCs. The iso-14-3-3 epsilon regulates viral replication and apoptosis. In contrast, 14-3-3 eta expression decreased (−2.4-fold) following co-activation. 14-3-3 gamma binds to the insulin-like growth factor receptor, which is involved in glucose metabolism. Data shows that PIF regulates and targets practically all members of this group, revealing a complex regulatory effect on cell survival and function.

We found that PIF targets protein disulfideisomerase/thioredoxin (PDI) and PDI A4 not only in the embryo but also in immune cells. These proteins act as chaperons preventing protein aggregation, and abnormal folding as well as (through thioredoxin) protection against oxidative stress. Complementing this PDI function, PIF also targets heat shock proteins (HSPs, HSP 90B-O, HSP 90B1, Iso2-HSPA, and HSP70A5) which beyond just protection also reduce cellular stress, protein misfolding and have critical functions required for cell survival. HSPs related genes are also regulated by PIF as well. Following immune activation the HSP90 group of genes increased while the HSP70 decreased implying possible protective auto-regulation. The integrated PIF targeting supports a protective role.

PIF Targets Myosin-9 and Thymosin-alpha-1: Role in Macrophage and Neutrophil Activation

The highest-ranking protein target was Myosin 9, which is involved in macrophage membrane protrusion and chemotaxis interacting with calmodulin, also a PIF target. PIF also targets Thymosin-apha-1, which interacts with Histone-H4 (PIF target) and aids in developing resistance against opportunistic and viral infections. PIF targets S100A8, which activates both leucocytes and macrophages. The respective gene EF hand calcium binding domain was downregulated at both 24 (−8.8-fold) and at 48h S100A8 (−2.2-fold) in naive PBMCs. The lymphocyte-specific protein 1 is involved in neutrophil activation and chemotaxis.

PIF Targets Myosin-9 and Thymosin-alpha-1: Role in Macrophage and Neutrophil Activation

Beyond protection, innate immune activation should preserve homeostasis. PIF targets activated macrophages. In this group, the highest-ranking protein target was Myosin-9, involved in macrophage membrane protrusion and chemotaxis interacting with calmodulin, also identified as a PIF target from the current data. PIF also targets Thymosin-alpha-1, which interacts with Histone-H4 (a PIF target), aiding in the development of resistance to opportunistic and viral infections. PIF targets protein S100A8, which activates both leukocytes and macrophages. The lymphocyte-specific protein 1 is involved in neutrophil activation and chemotaxis. Thus, the protein targets identified control both macrophages and neutrophils required for innate immune control.

PIF Targets Cytoskeleton Proteins: Role in Cell and Membrane Architecture

Actin, which has a major role in cell motility, was one of the highest-ranking proteins identified in the PIF binding study. Other PIF binding proteins identified were Tropomyosin alpha 1,3,4, which plays a major role in actin stabilization, and Tropomodulin, which regulates actin and is involved in maintaining membrane structure. PIF targets highly ranked actin-1 which plays a major role in cell motility. Connected to this data is that PIF also binds to Tropomyosin alpha 1,3, and 4 that play a major role in actin stabilization and also interacts with Talin-1. Talin-1 along with Tropomodulin (also targeted by PIF) is involved in attaching the cytoskeleton to the cell membrane acting to support membrane structure integrity. Notably, these data clearly point to PIF playing an important role in preserving the immune cells integrity.

PIF Binds to a Limited Number of Targets in CD4 and CD8 Cells: Role in Coagulation

Having demonstrated that PIF targets ˜5% of unstimulated T-cells, we further examined protein targets in these two lineages. Being cognizant of the fact that PIF binding without T-cell activation is low, we used a whole unit of blood for each CD4 or CD8 PIF target analysis. Using two different donors from PBMCs, CD4 or CD8 positive cells, respectively, were separated and extracted. This was followed by PIF-based affinity chromatography and mass spectrometry. We found that the number of targets in both T-cell sub-lineages was much lower <30% as compared with to the observed number of identified CD 14+ targets (Tables 16-18). Most protein targets >95% matched in all three cell preparations (CD14, CD4, CD8). When the protein targets in CD4 and CD8 were compared, in 21/24 cases they were matched (Table 19). These results support the reproducibility of the separation and method analysis in different subjects.

TABLE 16
PIF targets and regulates PDI and HSPs related proteins

| | | |
|---|---|---|
| Protein | Naïve | Activated |

| | |
|---|---|
| Protein disulfide-isomerase | −3.6 |
| Protein disulfide-isomerase A4 | |
| HSP 90-alpha(iso2)+ | |
| HSP 90-beta | 2.8 |
| HSP 70A5 | |
| Vimentin | −2.2 |
| PDI (P5) (gene) | 3.6 |
| Thioredoxin | −2.8 |
| HSP 90/B (gene) | 4.8 |
| HSPD1 (gene) | 2.2 |
| HSP 70AlA(gene) | −2.2 |
| HSP 701B(gene) | −2.6 |
| HSP 40 (gene) | −2.8 |
| HSP 70 B (gene) | −3 |

TABLE 17
PIF targets and regulates 14-3-3 scaffold proteins

| | | |
|---|---|---|
| Protein | Naïve | Activated |

| | |
|---|---|
| 14-3-3 protein eta | |
| 14-3-3 protein gamma | |
| 14-3-3 protein theta | |
| 14-3-3 protein zeta/delta | 2.8 |
| 14-3-3 protein beta/alpha (iso) | |
| 14-3-3 protein epsilon (iso) | −2.4 |

TABLE 18
PIF REGULATES INFLAMMATION IN CO-ACTIVATED PBMCs

| | | | |
|---|---|---|---|
| | Up or Down | | |
| Gene | Regulation | Title | Function |

Oxidative Stress Control

| | | | |
|---|---|---|---|
| HADHA | 7 | alpha subunit of | Oxidizes long chain fatty |
| | | mitochondrial | |
| PRDX3 | 3 | trituration protein | protection against oxidative |
| | | Peroxiredoxin | stress |
| LLT1 | 2 | C-type lectin domain | Prevents target cells from NK |
| | | | mediated lysis |
| TRX | −2.8 | thioredoxin | regulates oxygen radical |
| | | | formation |
| AL0X5 | −4 | Lipoxygenase | Synthesize leukotrienes |

Platelet Activation Control

| | | | |
|---|---|---|---|
| PECAM 1 | −2.4 | Platelet endothelial cell | Cell adhesion molecule |
| | | adhesion molecule | required for leukocyte |
| | | | transendothelial migration |
| ANXA5 | 2 | ANNEXIN-5 | Placental anticoagulant protein. |
| | | | Acts as a indirect inhibitor of |
| | | | thromboplastin |
| beta-thromboglobulin | −2.8 | pro-platelet basic | Potent chemoattractant & |
| (PPBP) | | protein | neutrophil activator |
| CD41B (ITGA2B) | −3.2 | Integrin | Important for fibrinogen |
| | | | formation PF4 −2.2 platelet |
| | | | factor 4 ITGB3 integrin B 3 -2,2 |

TABLE 19

| | | Protein | | |
|---|---|---|---|---|
| | | molecular | Protein | Number of |
| MS/MS sample | Protein name | weight | identification | unique |
| name | Sorted Alphabetically | (Da) | probability | peptides |
| 151061-CD-14 | 14-3-3 protein gamma | 28,303.10 | 100.00% | 2 |
| (Blue) Human | | | | |
| 151061-CD-14 | 14-3-3 protein zeta/delta | 27,745.90 | 100.00% | 4 |
| (Blue) Human | | | | |
| 151088-CD-8 | 14-3-3 protein zeta/delta | 28,037.30 | 99.80% | 2 |
| (Red) Human | | | | |
| 151088-CD-4 | 14-3-3 protein zeta/delta | 27,745.90 | 100.00% | 3 |
| (Green) Human | | | | |
| 151061-CD-14 | 60S ribosomal protein L6 | 32,729.30 | 100.00% | 3 |
| (Blue) Human | (Fragment) | | | |
| 151061-CD-14 | 78 kDa glucose-regulated protein | 72,334.70 | 100.00% | 4 |
| (Blue) Human | | | | |
| 151061-CD-14 | Acidic leucine-rich nuclear phosphoprotein 32 family | 28,586.10 | 100.00% | 3 |
| (Blue) Human | member A | | | |
| 151088-CD-8 | Acidic leucine-rich nuclear phosphoprotein 32 family | 28,586.10 | 99.80% | 2 |
| (Red) Human | member A | | | |
| 151088-CD-4 | Acidic leucine-rich nuclear phosphoprotein 32 family | 28,586.10 | 93.20% | 1 |
| (Green) Human | member A | | | |
| 151088-CD-4 | Actin, cytoplasmic 1 | 41,737.80 | 99.80% | 2 |
| (Green) Human | | | | |
| 151061-CD-14 | Actin, cytoplasmic 1 | 41,737.80 | 100.00% | 14 |
| (Blue) Human | | | | |
| 151088-CD-8 | Actin, cytoplasmic 1 | 41,737.80 | 100.00% | 3 |
| (Red) Human | | | | |
| 151061-CD-14 | Alpha-enolase | 47,170.20 | 100.00% | 2 |
| (Blue) Human | | | | |
| 151061-CD-14 | Apolipoprotein B receptor | 114,816.50 | 100.00% | 3 |
| (Blue) Human | | | | |
| 151061-CD-14 | Calmodulin | 16,838.00 | 100.00% | 5 |
| (Blue) Human | | | | |
| 151088-CD-8 | Calmodulin | 16,838.00 | 99.80% | 2 |
| (Red) Human | | | | |
| 151088-CD-4 | Calmodulin | 16,838.00 | 100.00% | 3 |
| (Green) Human | | | | |
| 151061-CD-14 | Calreticulin | 48,142.90 | 100.00% | 4 |
| (Blue) Human | | | | |
| 151088-CD-8 | Calreticulin | 48,142.90 | 99.80% | 2 |
| (Red) Human | | | | |
| 151088-CD-4 | Calreticulin | 48,142.90 | 100.00% | 4 |
| (Green) Human | | | | |
| 151088-CD-8 | Cartilage oligomeric matrix protein | 77,211.80 | 100.00% | 4 |
| (Red) Human | | | | |
| 151088-CD-4 | Cartilage oligomeric matrix protein | 79,694.20 | 100.00% | 10 |
| (Green) Human | | | | |
| 151061-CD-14 | Cofilin-1 | 18,503.20 | 100.00% | 3 |
| (Blue) Human | | | | |
| 151088-CD-8 | Cofilin-1 | 18,503.20 | 93.50% | 1 |
| (Red) Human | | | | |
| 151088-CD-4 | Cofilin-1 | 18,503.20 | 93.20% | 1 |
| (Green) Human | | | | |
| 151061-CD-14 | Filamin-A | 280,008.70 | 100.00% | 16 |
| (Blue) Human | | | | |
| 151061-CD-14 | Glucosidase 2 subunit beta | 59,425.80 | 100.00% | 6 |
| (Blue) Human | | | | |
| 151088-CD-4 | Glucosidase 2 subunit beta | 59,425.80 | 99.20% | 1 |
| (Green) Human | | | | |
| 151061-CD-14 | Hepatoma-derived growth factor | 26,788.60 | 99.90% | 2 |
| (Blue) Human | | | | |
| 151088-CD-8 | Hepatoma-derived growth factor | 22,964.00 | 93.50% | 1 |
| (Red) Human | | | | |
| 151088-CD-4 | Hepatoma-derived growth factor | 26,788.60 | 100.00% | 3 |
| (Green) Human | | | | |
| 151061-CD-14 | Histone H2A type 1-B/E | 14,108.10 | 100.00% | 2 |
| (Blue) Human | | | | |
| 151088-CD-4 | Histone H2Atype 1-B/E | 14,136.10 | 99.80% | 2 |
| (Green) Human | | | | |
| 151061-CD-14 | Isoform 1 of Vinculin | 123,801.30 | 100.00% | 5 |
| (Blue) Human | | | | |
| 151088-CD-4 | Isoform 11 of Titin | 0 | 100.00% | 2 |
| (Green) Human | | | | |
| 151061-CD-14 | Isoform 2 of Adenylyl cyclase-associated protein 1 | 51,901.60 | 100.00% | 3 |
| (Blue) Human | | | | |
| 151061-CD-14 | Isoform 2 of Heat shock protein HSP 90-alpha | 98,165.10 | 89.70% | 1 |
| (Blue) Human | | | | |
| 151088-CD-8 | Isoform 2 of Heat shock protein HSP 90-alpha | 84,663.20 | 100.00% | 2 |
| (Red) Human | | | | |
| 151088-CD-4 | Isoform 2 of Heat shock protein HSP 90-alpha | 84,663.20 | 100.00% | 4 |
| (Green) Human | | | | |
| 151061-CD-14 | Isoform 2 of Integrin alpha-lib | 113,376.70 | 99.90% | 2 |
| (Blue) Human | | | | |
| 151061-CD-14 | Isoform 2 of Polypyrimidine tract-binding protein 1 | 57,222.50 | 100.00% | 3 |
| (Blue) Human | | | | |
| 151061-CD-14 | Isoform 2 of Protein SET | 32,103.30 | 100.00% | 4 |
| (Blue) Human | | | | |
| 151088-CD-8 | Isoform 2 of Protein SET | 32,103.30 | 98.90% | 1 |
| (Red) Human | | | | |
| 151088-CD-4 | Isoform 2 of Protein SET | 32,103.30 | 100.00% | 3 |
| (Green) Human | | | | |
| 151061-CD-14 | Isoform 2 of Ras suppressor protein 1 | 31,542.20 | 100.00% | 3 |
| (Blue) Human | | | | |
| 151061-CD-14 | Isoform 4 of Latent-transforming growth factor beta- | 186,787.30 | 100.00% | 4 |
| (Blue) Human | binding protein 1 | | | |
| 151061-CD-14 | Isoform Short of 14-3-3 protein beta/alpha | 27,850.80 | 100.00% | 2 |
| (Blue) Human | | | | |
| 151061-CD-14 | Lysozyme C | 16,536.90 | 100.00% | 4 |
| (Blue) Human | | | | |
| 151061-CD-14 | Myeloid cell nuclear differentiation antigen | 45,837.00 | 100.00% | 5 |
| (Blue) Human | | | | |
| 151061-CD-14 | Myosin regulatory light chain 12A | 19,795.30 | 100.00% | 4 |
| (Blue) Human | | | | |
| 151061-CD-14 | Myosin-9 | 226,537.50 | 100.00% | 36 |
| (Blue) Human | | | | |
| 151061-CD-14 | Neuroblast differentiation-associated protein AHNAK | 629,104.40 | 100.00% | 2 |
| (Blue) Human | | | | |
| 151061-CD-14 | Nuclease-sensitive element-binding protein 1 | 42,015.90 | 100.00% | 4 |
| (Blue) Human | (Fragment) | | | |
| 151088-CD-8 | Nuclease-sensitive element-binding protein 1 | 35,923.80 | 93.50% | 1 |
| (Red) Human | (Fragment) | | | |
| 151061-CD-14 | Nucleolin | 76,615.90 | 100.00% | 3 |
| (Blue) Human | | | | |
| 151088-CD-8 | Nucleolin | 76,615.90 | 93.50% | 1 |
| (Red) Human | | | | |
| 151088-CD-4 | Nucleolin | 76,615.90 | 99.90% | 2 |
| (Green) Human | | | | |
| 151061-CD-14 | Perilipin-3 | 45,802.10 | 99.90% | 2 |
| (Blue) Human | (Fragment) | | | |
| 151061-CD-14 | Platelet factor 4 | 10,845.50 | 100.00% | 5 |
| (Blue) Human | | | | |
| 151061-CD-14 | Proteasome activator complex subunit 1 | 28,723.90 | 99.90% | 2 |
| (Blue) Human | | | | |
| 151061-CD-14 | Protein S100-A8 | 10,835.00 | 100.00% | 2 |
| (Blue) Human | | | | |
| 151061-CD-14 | Pyruvate kinase isozymes M1/M2 | 57,937.50 | 100.00% | 4 |
| (Blue) Human | | | | |
| 151088-CD-4 | Serine/arginine-rich-splicing factor 1 | 28,329.70 | 100.00% | 2 |
| (Green) Human | | | | |
| 151061-CD-14 | Serine/arginine-rich-splicing factor 1 | 27,745.10 | 99.80% | 1 |
| (Blue) Human | | | | |
| 151088-CD-8 | Serine/arginine-rich-splicing factor 1 | 28,329.70 | 93.50% | 1 |
| (Red) Human | | | | |
| 151061-CD-14 | Serine/arginine-rich-splicing factor 2 | 25,477.10 | 100.00% | 3 |
| (Blue) Human | | | | |
| 151088-CD-4 | Serine/arginine-rich-splicing factor 2 | 25,477.10 | 93.20% | 1 |
| (Green) Human | | | | |
| 151061-CD-14 | Serum albumin | 69,366.90 | 99.80% | 2 |
| (Blue) Human | | | | |
| 151088-CD-8 | Serum albumin | 69,366.90 | 100.00% | 3 |
| (Red) Human | | | | |
| 151088-CD-4 | Serum albumin | 69,366.90 | 100.00% | 4 |
| (Green) Human | | | | |
| 151061-CD-14 | Serum deprivation-response protein | 47,172.90 | 100.00% | 3 |
| (Blue) Human | | | | |
| 151088-CD-8 | Serum deprivation-response protein | 47,172.90 | 100.00% | 2 |
| (Red) Human | | | | |
| 151088-CD-4 | Serum deprivation-response protein | 47,172.90 | 99.90% | 2 |
| (Green) Human | | | | |
| 151061-CD-14 | Talin-1 | 269,765.10 | 100.00% | 27 |
| (Blue) Human | | | | |
| 151088-CD-8 | Talin-1 | 269,765.10 | 97.80% | 1 |
| (Red) Human | | | | |
| 151088-CD-4 | Thrombin light chain | 65,408.20 | 100.00% | 3 |
| (Green) Human | | | | |
| 151061-CD-14 | Thrombospondin-1 | 129,381.70 | 100.00% | 8 |
| (Blue) Human | | | | |
| 151088-CD-8 | Thrombospondin-1 | 129,381.70 | 100.00% | 7 |
| (Red) Human | | | | |
| 151088-CD-4 | Thrombospondin-1 | 129,381.70 | 100.00% | 6 |
| (Green) Human | | | | |
| 151088-CD-8 | Thrombospondin-4 | 96,005.30 | 100.00% | 3 |
| (Red) Human | | | | |
| 151088-CD-4 | Thrombospondin-4 | 96,005.30 | 100.00% | 3 |
| (Green) Human | | | | |
| 151061-CD-14 | Thymosin alpha-1 | 12,073.40 | 100.00% | 6 |
| (Blue) Human | | | | |
| 151088-CD-8 | Thymosin alpha-1 | 12,073.40 | 99.80% | 2 |
| (Red) Human | | | | |
| 151088-CD-4 | Thymosin alpha-1 | 12,073.40 | 100.00% | 4 |
| (Green) Human | | | | |
| 151061-CD-14 | Tropomyosin alpha-3 chain | 29,033.30 | 100.00% | 2 |
| (Blue) Human | | | | |
| 151061-CD-14 | Tropomyosin alpha-4 chain | 28,522.40 | 100.00% | 7 |
| (Blue) Human | | | | |
| 151088-CD-8 | Tropomyosin alpha-4 chain | 28,522.40 | 100.00% | 3 |
| (Red) Human | | | | |
| 151088-CD-4 | Tropomyosin alpha-4 chain | 28,522.40 | 100.00% | 4 |
| (Green) Human | | | | |
| 151088-CD-8 | Tropomyosin alpha-4 chain | 28,522.40 | 100.00% | 3 |
| (Red) Human | | | | |
| 151061-CD-14 | Tubulin alpha- 1C chain | 50,135.70 | 100.00% | 3 |
| (Blue) Human | | | | |
| 151061-CD-14 | 14-3-3 protein gamma | 28,303.10 | 100.00% | 2 |
| (Blue) Human | | | | |
| 151088-CD-8 | 14-3-3 protein zeta/delta | 28,037.30 | 99.80% | 2 |
| (Red) Human | | | | |
| 151088-CD-8 | Acidic leucine-rich nuclear phosphoprotein 32 family | 28,586.10 | 99.80% | 2 |
| (Red) Human | member A | | | |
| 151088-CD-4 | Actin, cytoplasmic 1 | 41,737.80 | 99.80% | 2 |
| (Green) Human | | | | |
| 151061-CD-14 | Alpha-enolase | 47,170.20 | 100.00% | 2 |
| (Blue) Human | | | | |
| 151088-CD-8 | Calmodulin | 16,838.00 | 99.80% | 2 |
| (Red) Human | | | | |
| 151088-CD-8 | Calreticulin | 48,142.90 | 99.80% | 2 |
| (Red) Human | | | | |
| 151061-CD-14 | Hepatoma-derived growth factor | 26,788.60 | 99.90% | 2 |
| (Blue) Human | | | | |
| 151061-CD-14 | Histone H2A type 1-B/E | 14,108.10 | 100.00% | 2 |
| (Blue) Human | | | | |
| 151088-CD-4 - | Histone H2A type 1-B/E | 14,136.10 | 99.80% | 2 |
| (Green) Human | | | | |
| 151088-CD-4 | Isoform 11 of Titin | 0 | 100.00% | 2 |
| (Green) Human | | | | |
| 151088-CD-8 | Isoform 2 of Heat shock protein HSP 90-alpha | 84,663.20 | 100.00% | 2 |
| (Red) Human | | | | |
| 151061-CD-14 | Isoform 2 of Integrin alpha-lib | 113,376.70 | 99.90% | 2 |
| (Blue) Human | | | | |
| 151061-CD-14 | Isoform Short of 14-3-3 protein beta/alpha | 27,850.80 | 100.00% | 2 |
| (Blue) Human | | | | |
| 151061-CD-14 | Neuroblast differentiation-associated protein AHNAK | 629,104.40 | 100.00% | 2 |
| (Blue) Human | | | | |
| 151088-CD-4 | Nucleolin | 76,615.90 | 99.90% | 2 |
| (Green) Human | | | | |
| 151061-CD-14 | Perilipin-3 (Fragment) | 45,802.10 | 99.90% | 2 |
| (Blue) Human | | | | |
| 151061-CD-14 | Proteasome activator complex subunit 1 | 28,723.90 | 99.90% | 2 |
| (Blue) Human | | | | |
| 151061-CD-14 | Protein S100-A8 | 10,835.00 | 100.00% | 2 |
| (Blue) Human | | | | |
| 151088-CD-4 | Serine/arginine-rich-splicing factor 1 | 28,329.70 | 100.00% | 2 |
| (Green) Human | | | | |
| 151061-CD-14 | Serum albumin | 69,366.90 | 99.80% | 2 |
| (Blue) Human | | | | |
| 151088-CD-8 | Serum deprivation-response protein | 47,172.90 | 100.00% | 2 |
| (Red) Human | | | | |
| 151088-CD-4 | Serum deprivation-response protein | 47,172.90 | 99.90% | 2 |
| (Green) Human | | | | |
| 151088-CD-8 | Thymosin alpha-1 | 12,073.40 | 99.80% | 2 |
| (Red) Human | | | | |
| 151061-CD-14 | Tropomyosin alpha-3 chain | 29,033.30 | 100.00% | 2 |
| (Blue) Human | | | | |
| 151088-CD-4 | Tubulin alpha-lC chain | 50,135.70 | 99.80% | 2 |
| (Green) Human | | | | |
| 151088-CD-4 | Acidic leucine-rich nuclear phosphoprotein 32 family | 28,586.10 | 93.20% | 1 |
| (Green) Human | member A | | | |
| 151088-CD-8 | Cofilin-1 | 18,503.20 | 93.50% | 1 |
| (Red) Human | | | | |
| 151088-CD-4 | Cofilin-1 | 18,503.20 | 93.20% | 1 |
| (Green) Human | | | | |
| 151088-CD-4 | Glucosidase 2 subunit beta | 59,425.80 | 99.20% | 1 |
| (Green) Human | | | | |
| 151088-CD-8 | Hepatoma-derived growth factor | 22,964.00 | 93.50% | 1 |
| (Red) Human | | | | |
| 151061-CD-14 | Isoform 2 of Heat shock protein HSP 90-alpha | 98,165.10 | 89.70% | 1 |
| (Blue) Human | | | | |
| 151088-CD-8 | Isoform 2 of Protein SET | 32,103.30 | 98.90% | 1 |
| (Red) Human | | | | |
| 151088-CD-8 | Nuclease-sensitive element-binding protein 1 | 35,923.80 | 93.50% | 1 |
| (Red) Human | (Fragment) | | | |
| 151088-CD-8 | Nucleolin | 76,615.90 | 93.50% | 1 |
| (Red) Human | | | | |
| 151061-CD-14 | Serine/arginine-rich-splicing factor 1 | 27,745.10 | 99.80% | 1 |
| (Blue) Human | | | | |
| 151088-CD-8 | Serine/arginine-rich-splicing factor 1 | 28,329.70 | 93.50% | 1 |
| (Red) Human | | | | |
| 151088-CD-4 | Serine/arginine-rich-splicing factor 2 | 25,477.10 | 93.20% | 1 |
| (Green) Human | | | | |
| 151088-CD-8 | Talin-1 | 269,765.10 | 97.80% | 1 |
| (Red) Human | | | | |
| 151088-CD-8 | Tubulin alpha-1C chain | 50,135.70 | 93.50% | 1 |
| (Red) Human | | | | |

This result is very important since to assure data reproducibility in all cases the same number of PBMCs were isolated. This avoided any possibility of low detection due to the low binding present in unstimulated T-cells. The cartilage oligomeric matrix protein common to CD4 and CD8 cells was not found in CD14 cells. This protein mainly but not exclusively in an extracellular location is involved in arthritis and is as part of the thrombospondin family; similarly, thrombospondin-4 was also found only in CD4 and CD8 cells. Thrombin light chain was noted only in CD4 cells. It has a major role in converting fibrinogen to fibrin, and is involved in the activation of several factors in the coagulation cascade. The serine/arginine-rich-splicing factor 2 related to pre-mRNA splicing was common for CD 14+ and CD4+ lineage. The Talinl cytoskeletal protein that links the cytoskeleton with the cell membrane was common to CD14+ and CD8+ cells. This protein is involved in neutrophil rolling. The data implies that most PIF targets are shared by the CD14, CD8, CD4 lineages.

PIF Targets Systemic Immunity In Vivo

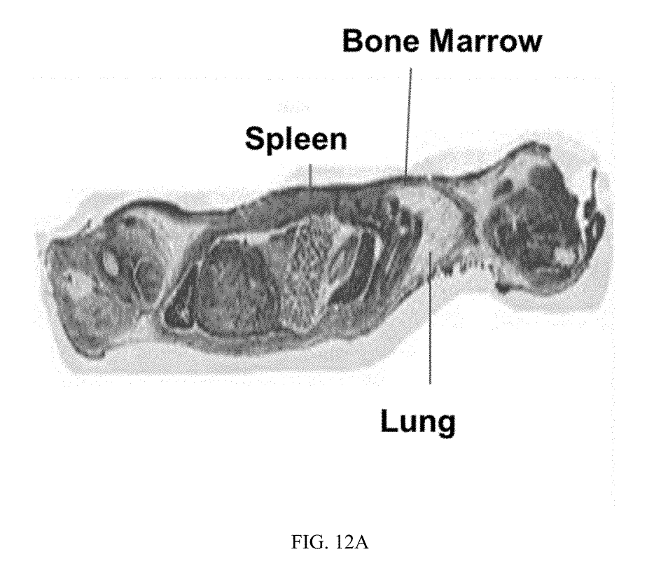

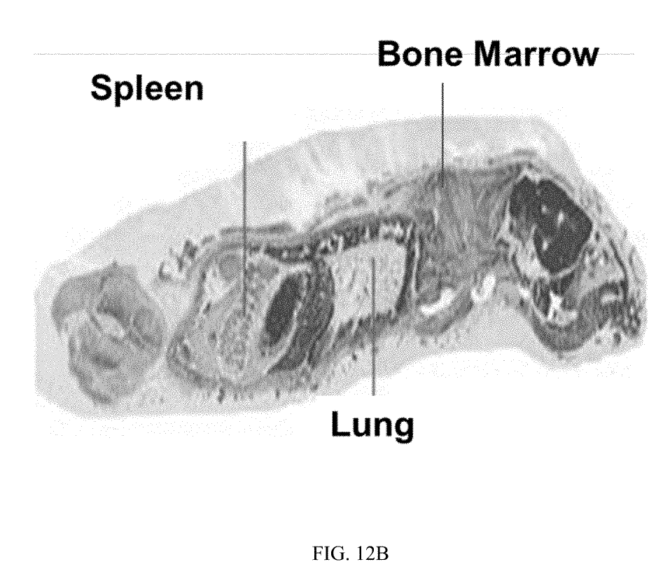

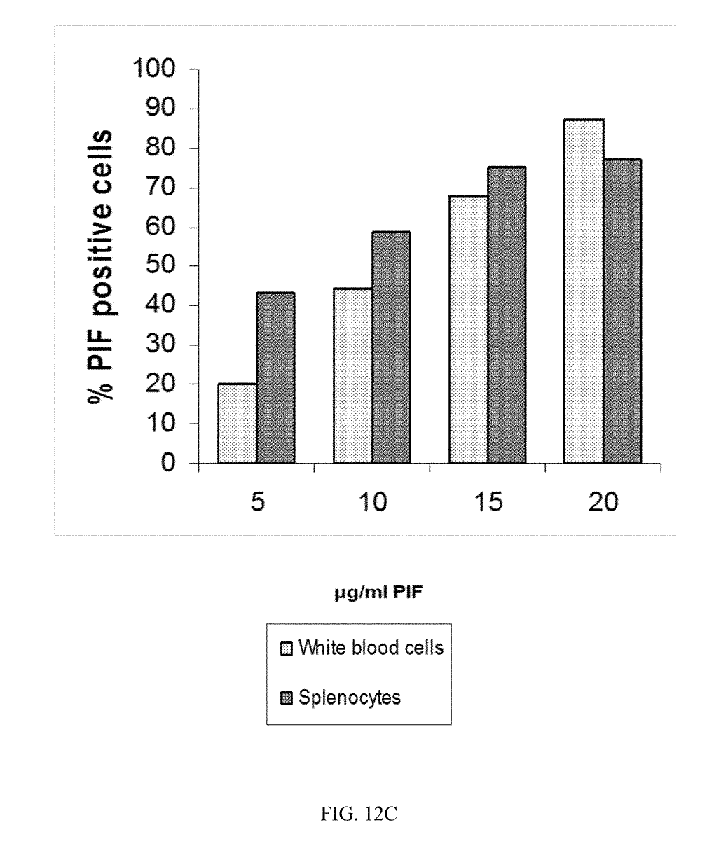

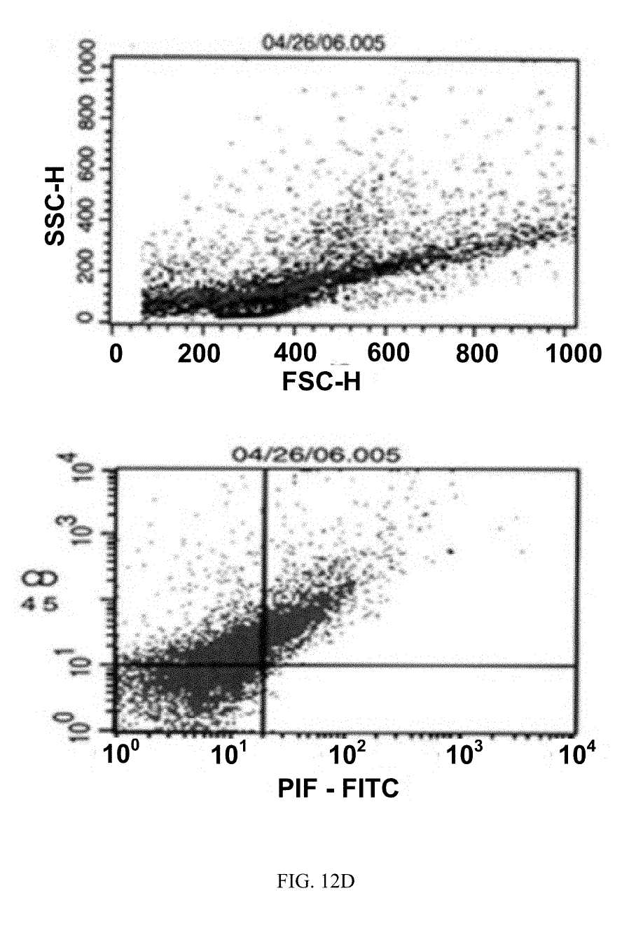

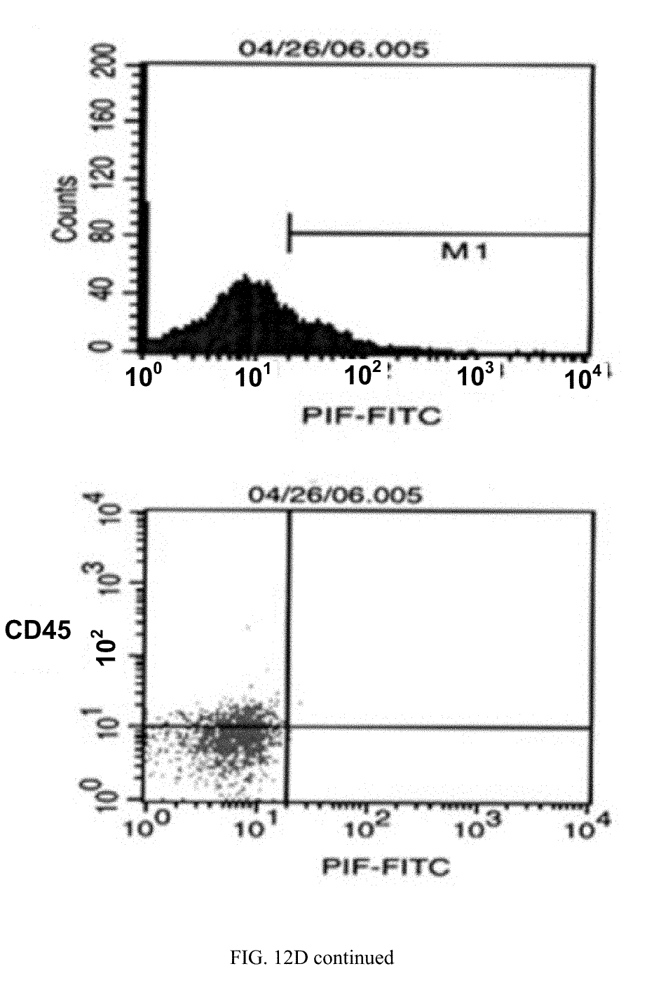

In vitro cultured PIF targets the human immune system. However whether this also occurs in vivo has not been established. To determine whether PIF targets the immune system in the intact mouse, FITC-PIF was injected intravenously (IV) or intra-peritoneally (IP) followed by sacrifice 5 min and 30 min later, respectively. Global distribution of PIF within the body was analyzed through imaging. Data revealed that within 5 min a major uptake of the labeled PIF was noted within the spleen and bone marrow (FIGS. 12A and 12B). A major accumulation of the labeled peptide was observed in the kidney, reflecting a rapid clearance. Following IP injection, the uptake and clearance was slower than following IV administration, as expected. This indicates that the kidney is the major site of PIF clearance.

FITC-PIF Binds to Circulating CD45+ Immune Cells

To further confirm that PIF directly targets the immune system in vivo, we examined FITC-PIF interaction with circulating CD45+ cells in naive mice. These are regulators of T- and B-cell antigen receptor signaling. Using two-color flow cytometry, we found that FITC-sPIF incubated with isolated circulating mouse white blood cells binds up to 25% of those cells when exposed to 12.5-50 μg/mL FITC-PIF, with no differences found among the tested peptide concentrations, 23-25%, respectively. This indicates that in naive mice, PIF targets are limited, contrary to what is observed when immunity is activated. The direct PIF-spleen and immune cell interaction was further confirmed in in vitro studies (FIG. 12C). The binding to ex vivo CD45 cells was also confirmed (FIG. 12D). This confirmed that PIF targets the systemic immunity despite its short circulating half-life.

PIF Targets 14-3-3eta Protein Bioinformatics Conformation

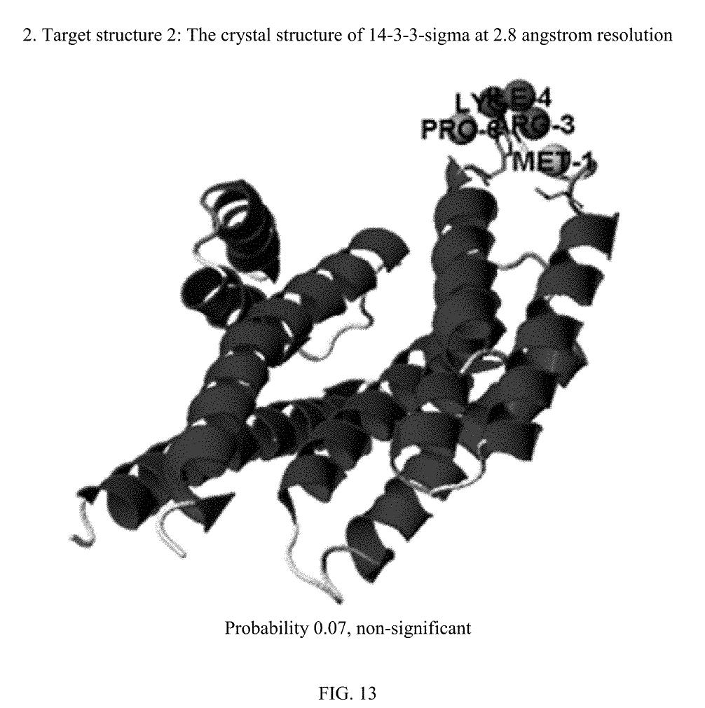

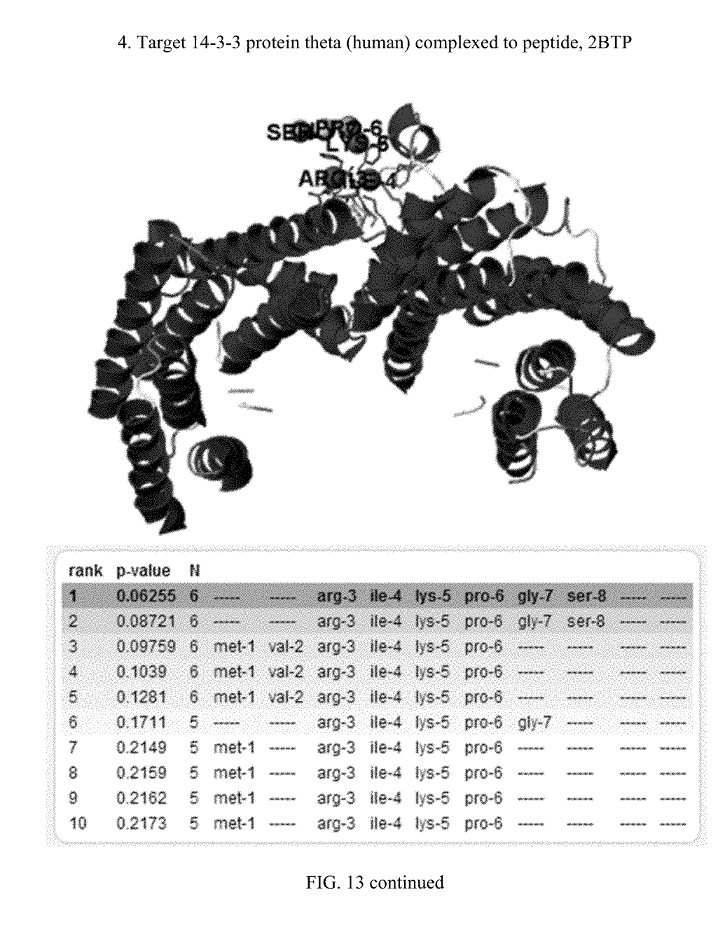

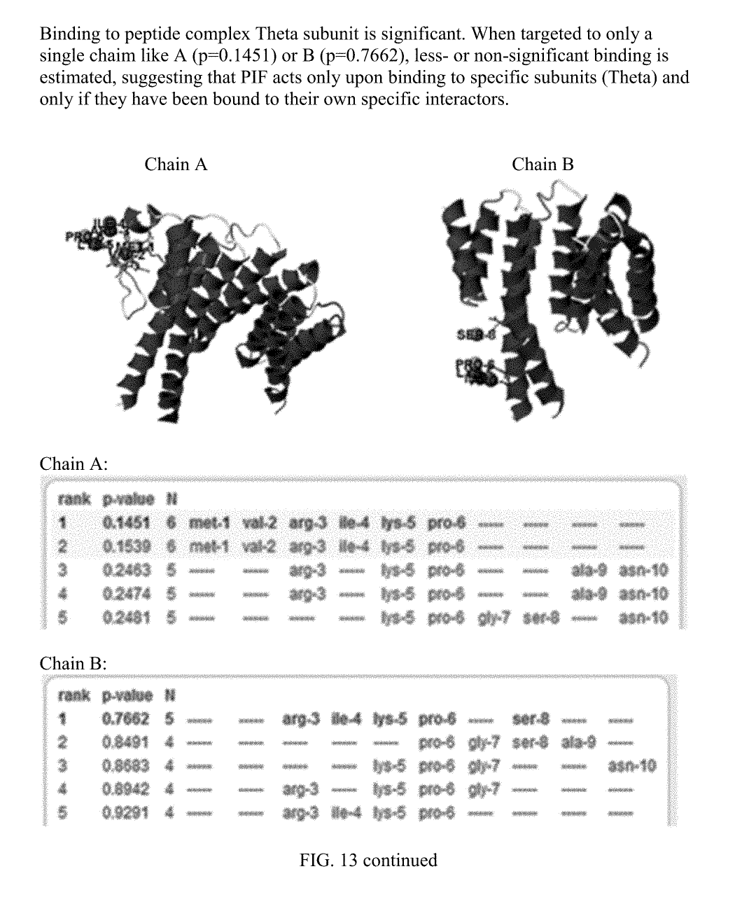

14-3-3 proteins are known to interact with a large number of targets due to their scaffolding structure and flexibility. In order further define the possible intimate interaction between these proteins and PIF, we examined its interaction with different proteins using bioinformatics. The data revealed that the only member of the group that was significantly interacting with PIF was 14-3-3eta (FIG. 13). Interestingly, the significant binding was present only when the protein was complexed with a peptide 2BTP. Analysis of binding to the other 14-3-3 proteins was less significant, This implies that the 14-3-3 proteins, due to their multiple binding partners, may interact with PIF to regulate the immune response.

SPR Analysis: Evidence for PIF's Direct Action on PBMCs, While Not Engaging with LPS, CD14 or Downstream TLR4-MD2

We have previously observed that PIF prevents LPS (lipopolysaccharide-bacterial antigen)-induced nitric oxide (NO) production by macrophages 17, 18 260 Therefore, it was important to whether the inhibitory action is due to direct peptide-LPS interaction. Binding between PIF and rough (Ra LPS) or smooth (O55:B5 LPS) LPS was examined using surface plasmon resonance (SPR) by passing over the PIF attached sensor (FIGS. 7A and 7B). No LPS (ligand) and PIF-sensor interaction was observed at all concentrations tested. Therefore we examined whether PIF targets the CD 14 receptor or its immediate downstream TLR4-MD2 ligands. The SPR based analysis showed that PIF neither targets the receptor nor its immediate down-stream mediators even when tested at high concentrations (FIG. 8A). Lack of interaction, with TLR4-MD2 surfaces at high concentration (0.5 mM) of PIF, was also confirmed (FIG. 8B). PIF therefore acts through cognate cellular process, involving specific targets, rather than through secondary interaction with activating agents.

PIF Effect is Likely Dependent on TLR4 Downstream Proteins

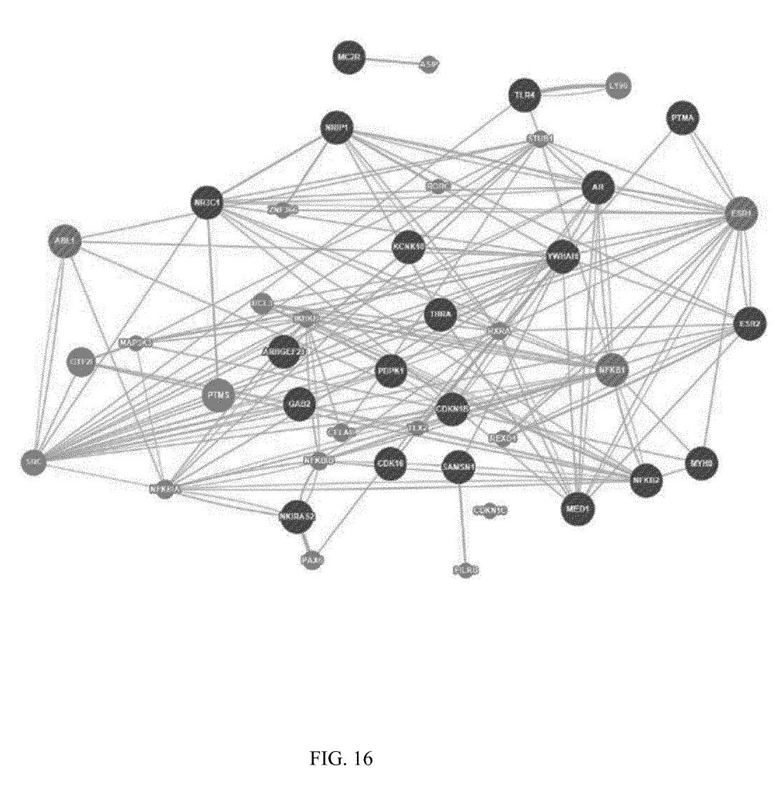

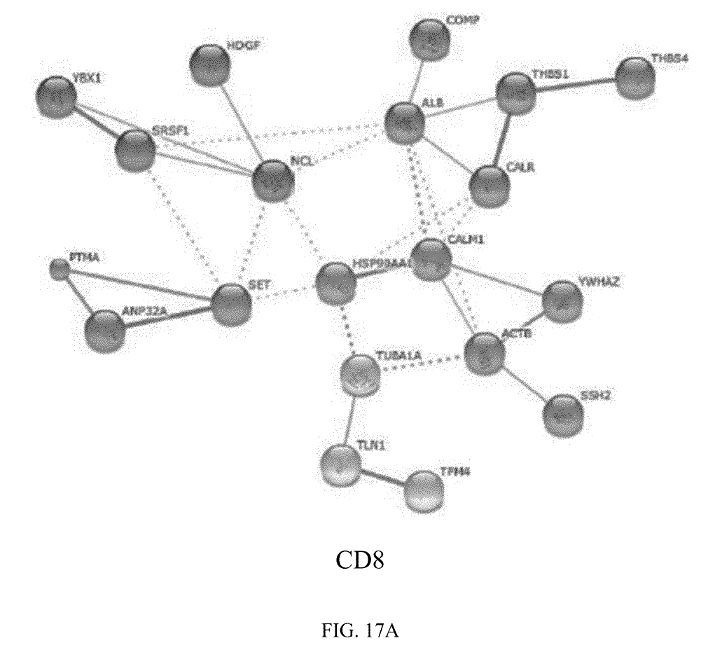

We already showed that PIF does not bind TLR4 however TLR4 siRNA blocked the peptide effect. TLR4 are mostly expressed by CD 14+ cells therefore PIF targets identified in these cells enabled to examine proteins involved in transduction of TLR4 effect (FIG. 16). The data showed three major proteins targeted by PIF Myosin 9, Thymosin a1 involved in immune activation and 14-3-3eta that are significant for TLR4 action. Therefore disruption of any of those signaling proteins by the TLR4 inhibitor may impair PIF's ability to control the inflammatory response.

Additional Role in Immune Cell Targeting: PIF Targets in CD4+ and 281 CD8+ Cells are Highly Correlated to the CD14+ Targets

It was important to examine protein targets in CD4+ and CD8+ (unstimulated) lymphocyte sub-lineages as compared to CD 14+ targets. Overall, the number of targets in both T-cell sub-lineages was much lower (<30% as compared with CD 14+ targets), (Tables 12 and 13). Most of the PIF targeted proteins were highly conserved with >95% matching in all three cell preparations; (CD14+, CD4+, CD8+). The CD4+ and CD8+ targets in 21/24 cases matched proteins identified were identical. This provides strong support that the separation and method analysis is reproducible in different subjects since, in all cases, the same number of PBMCs was isolated. This avoided any possibility of limited detection due to the low binding present in unstimulated T-cells. It also confirmed the robustness of the method of protein identification. However, as data shows the protein expression in those lineages are much lower however, a number of critical proteins identified in these lineages are not seen in CD 14 cells described below.

Discussion

The immune system is complex and requires constant adaptation to exposed scenarios. PIF has been shown to regulate both innate and adaptive immunity, and has shown in vivo efficacy in several diverse preclinical immune disorders. PIF interaction must be direct, specific and multi-targeted. The above-described experiments show that PIF directly targets the immune system, and interacts with regulatory T-cells FoxP3+ required for immune surveillance. In addition, PIF targets several proteins in unstimulated CD 14 cells, which are mostly shared by CD4 and CD8 cells. In line with its role in protection against oxidative stress and protein misfolding, PIF interacts with vimentin, PDI/Thioredoxin, HSPs and interestingly with several 14-3-3 proteins that have a critical role in immune function. Further, PIF's interaction with myosin 9 and thymosin-alpha-1 supports regulation of the immune response. The binding to several cytoskeleton proteins indicates involvement in cell motility and membrane architecture. Finally, in vivo data confirms that shortly after injection PIF is taken up rapidly by the immune system. Overall, both in vitro and in vivo data support direct and targeted PIF-immune system interaction.

A key element in the immune response is understanding how a regulatory agent influences the immune system. We have used approaches to address this question: cell-free, cell-based, identification of interacting targets, and in vivo evidence.

LPS is a major activator of the immune system derived from bacteria, which has a complex action on the cell. It mainly interacts with the CD 14 receptor which, further transduces the ligand induced activity through a TLR4-MD2 downstream effect. However, LPS has also a TLR4-independent action, entering the cell and possibly activating the inflammasome. Although PIF regulates LPS-induced immune function in vitro as well as in vivo, PIF action is independent of binding to the ligand, thus supporting a clear cell-based action. Since PIF targets CD14+ cells in unstimulated cells where LPS mainly binds, the peptide does not bind to the receptor or its immediate downstream pathway. Such data supports the view for PIF cell-based action where the interference with LPS action has to be exerted by targets present within the immune cell itself, possibly downstream in the TLR-4 pathway.

PIF interaction with the adoptive immune system was examined under unstimulated conditions. Recognizing that PIF binding to those naive cells is ˜5%, it was important to determine the nature of this interaction, especially since in earliest stages of pregnancy when the embryo is a small antigen it would not lead forcibly to activation of the immune system. However, it appears that this is not the case, since there is an increase in pro-tolerogenic regulatory T-cells (CD4/CD25/FoxP3+) already prior to implantation. Our data support the notion that despite the low binding, PIF targets those cells that specifically express the FoxP3 activation marker. Such data indicates that PIF may be instrumental in increasing the expression of those cells' markers shortly post-fertilization. PIF is secreted by viable embryos and is detected in the maternal circulation shortly post-insemination and prior to implantation. This data also supports the notion that PIF action could involve interaction with this specific critical cell type in a non-pregnant setting, and thus may contribute to the earliest maternal recognition of pregnancy.

The data showing that PIF targets directly both CD 14+ and T-cells prompted examination of the specific targets involved. Following a validated PIF-based affinity chromatography method, we found that due to its flexible structure, PIF interacts with multiple proteins. Interestingly, targets identified in the three lineages mostly matched, although CD14 had a three-fold higher number of targets. This is remarkable since cells were ucnvcu nuin mice uiiiciciu uuuuis. in mic wim rir action several proieuis invoiveu in oxidative stress were identified, with vimentin and PDI/Thioredoxin among them. PDI/Thioredoxin is critical for protection against oxidative stress and has been shown to be upregulated in vivo by PIF in the pancreas in a juvenile diabetes model. Beyond targeting HSPs, which complement greatly PDI action in protection against cellular stress, 14-3-3 proteins were also identified as a major protein group of PIF-interactors. This group represented 10% of all PIF targets and practically covered all members of this class of proteins.

The interaction with proteins such as Myosin 9 and Thymosin-alpha-1 support the view that PIF is involved not only in protection, but also in immune regulation and activation.

The cytoskeleton plays a critical role in cell function and survival. It preserves the cell architecture and membrane integrity, and enables cell mobility. By interacting with these diverse proteins, PIF helps control cell migration.

Overall, the diversity of PIF protein-binding candidates provides evidence that PIF interaction with the immune system is robust and in certain cases PIF not only binds to but also regulates the same genes' expression. Thus, it closes the loop between binding and the action on the same gene. Whether this is exerted through a direct feedback or alternatively by indirect mechanisms is uncertain.

The PIF affinity chromatography method utilized for the studies described above was followed by semi-quantitative mass spectrometry. This method was validated where the Biotin-PIF binding to selective fractions of mouse embryo extracts was compared with the PIF-based affinity chromatography results. The data showed a high concordance, which was followed by identifying the RIKP active site of the PIF peptide as targeting the binding sites of PDI and HSPs. There was a 63% concordance with respect to the targets between the two distinct tissues and species, strongly support the validity of these observations. The difference in ranking of the proteins between the adult and embryo further confirms the validity of the obtained data.

In vitro observation requires in vivo confirmation. In this case, demonstrating that PIF is directly taken up by the systemic immune system within minutes of administration confirmed the PIF-immune cell interaction in a relevant murine model. It also showed that the portion of PIF that is not bound to the spleen and bone marrow rapidly reaches the kidney. PIF reaches the brain to target the microglia; however, by that time it has already been cleared from the circulation. This is further supported since for FDA-mandated toxicology studies injections of PIF to both mice and dogs at very high concentration (4000× higher than planned for clinical studies) was cleared within a couple of hours from the circulation when analyzed with a validated LC/MS/MS method.

The data described above support the view that by multi-targeting, PIF regulates the immune response ranging from cell protection to immune activation and cell structure.

Maternal immunity is continuously exposed to environmental pathogens, and a suppressed state would harm both host and progeny. Paradoxically, several autoimmune disorders may improve during pregnancy unless the disease is severe, but previous poor pregnancy outcome contributes to later disease. Thus, the fetus has to interact in synergy, and PIF interaction with adaptive immunity CD3+ cells is enhanced in pregnancy.

FITC-PIF Binding to CD3 and CD45 Cells is Affected by Endometriosis Sera

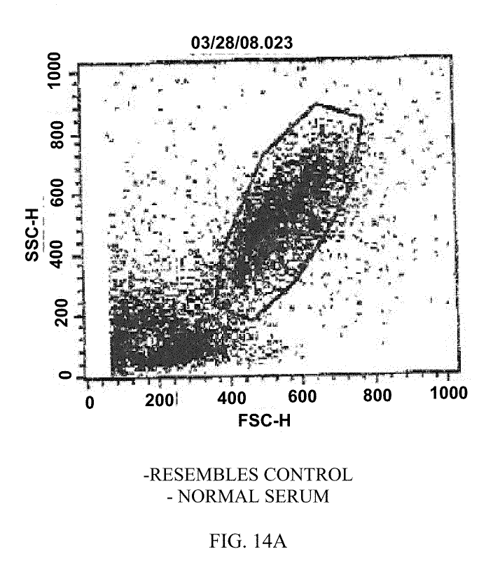

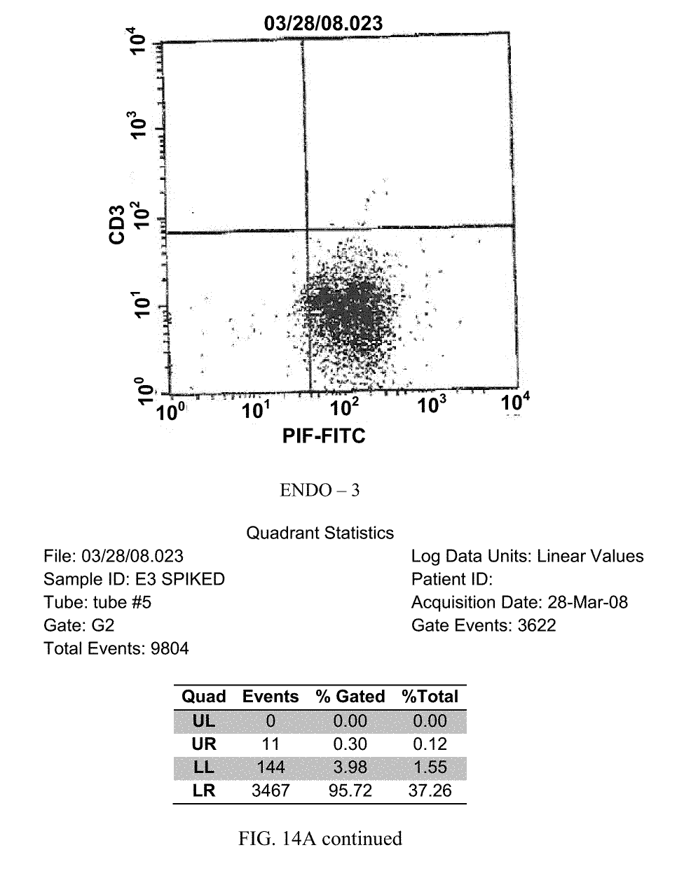

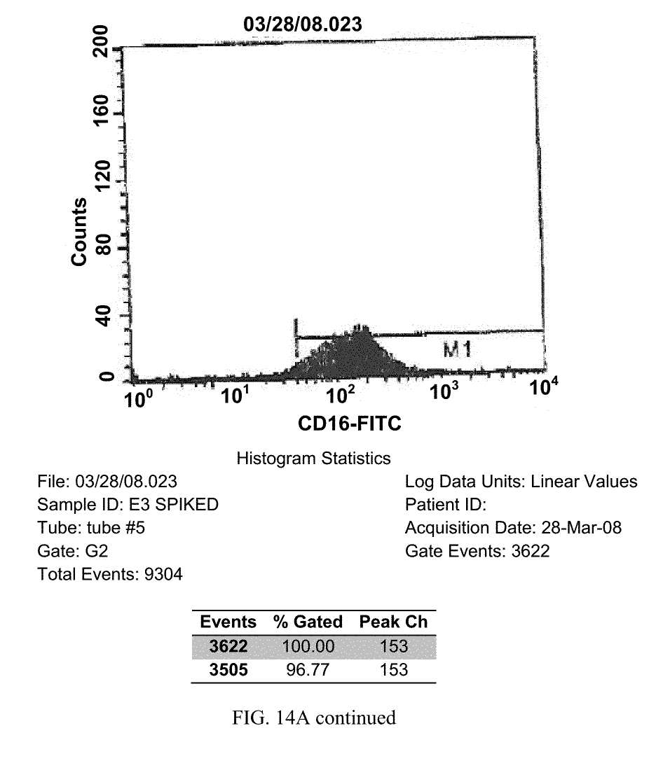

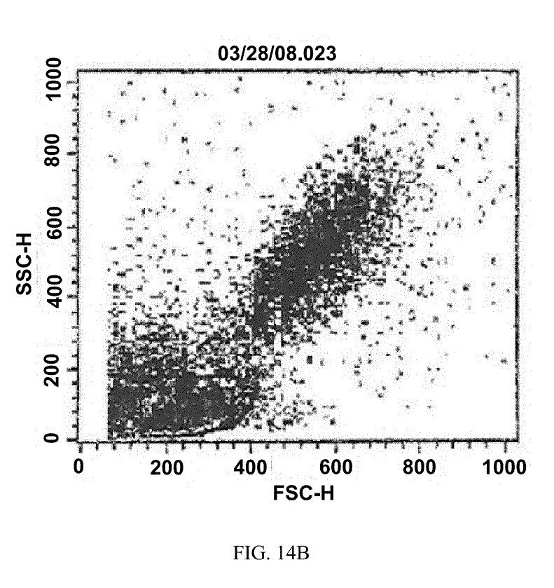

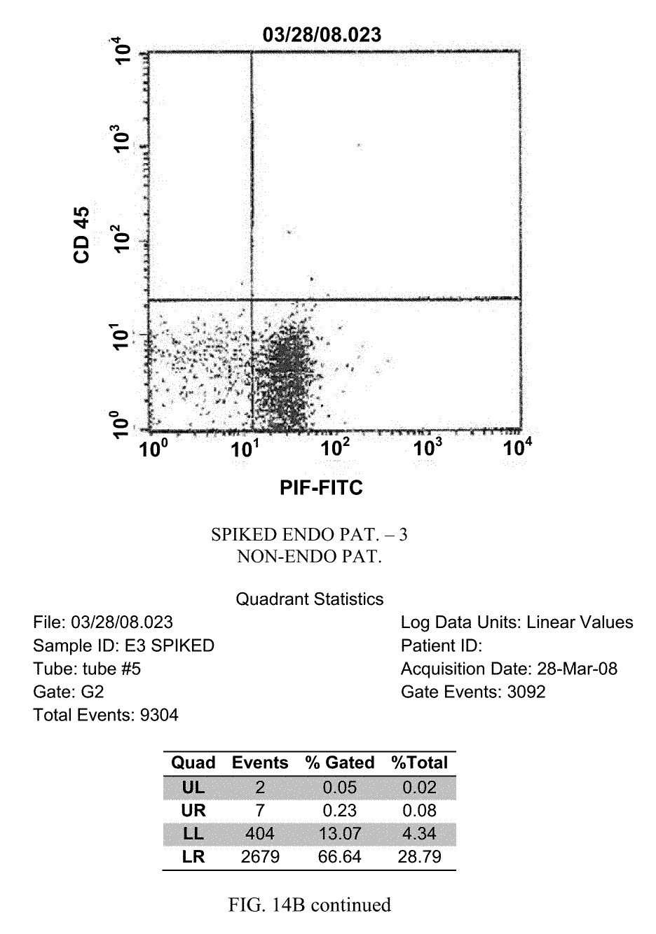

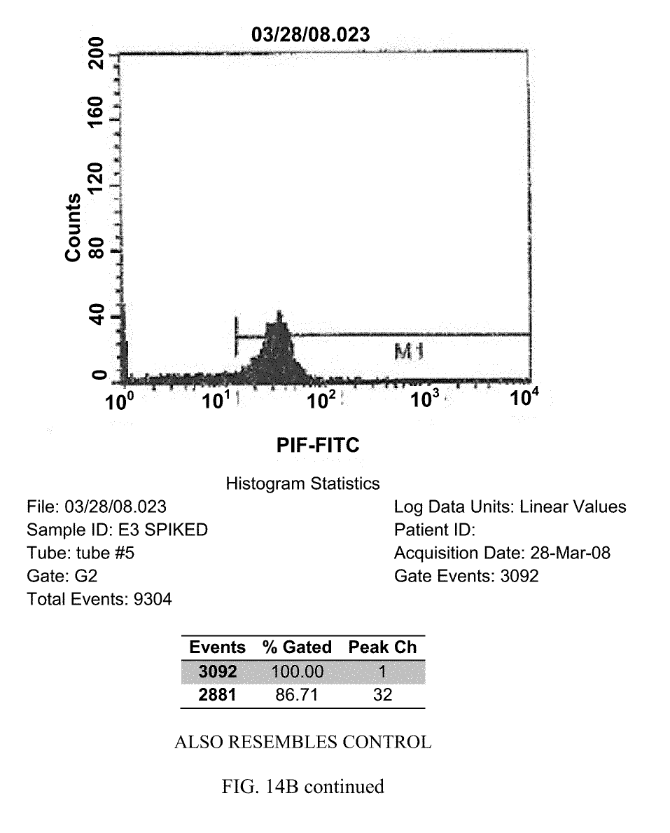

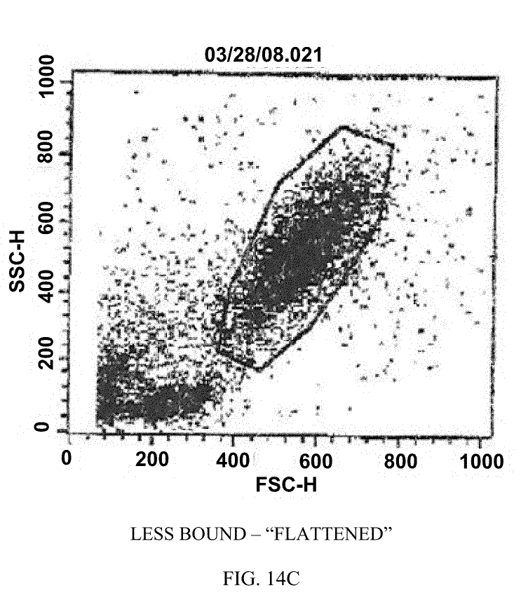

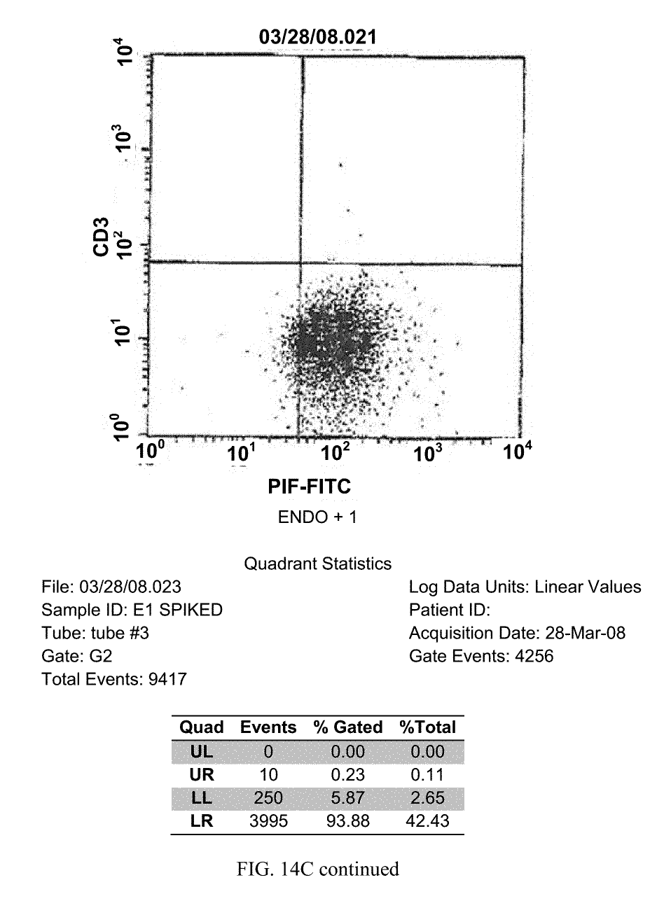

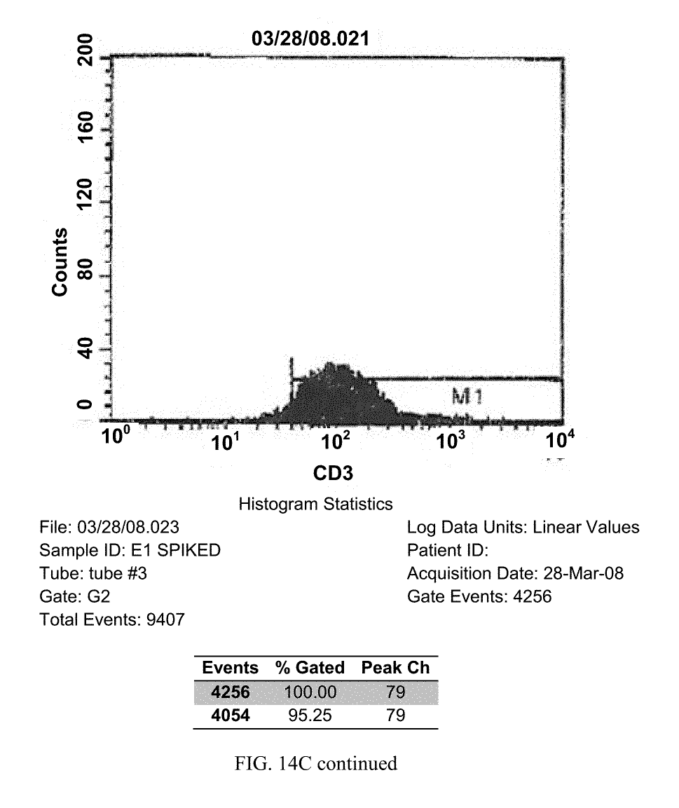

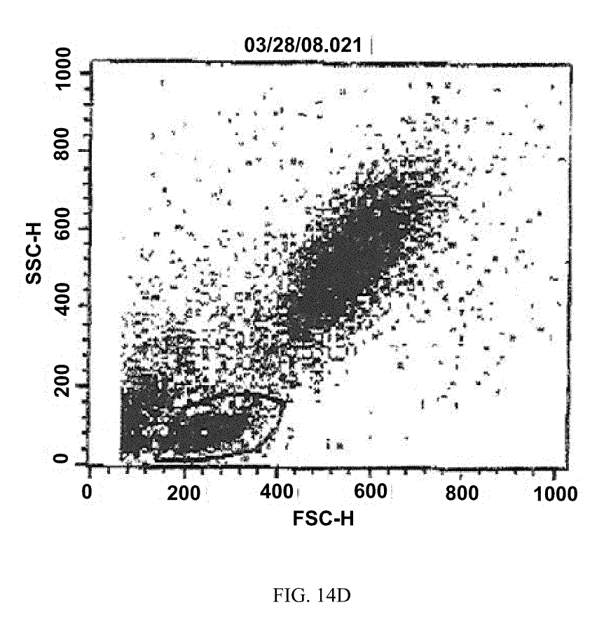

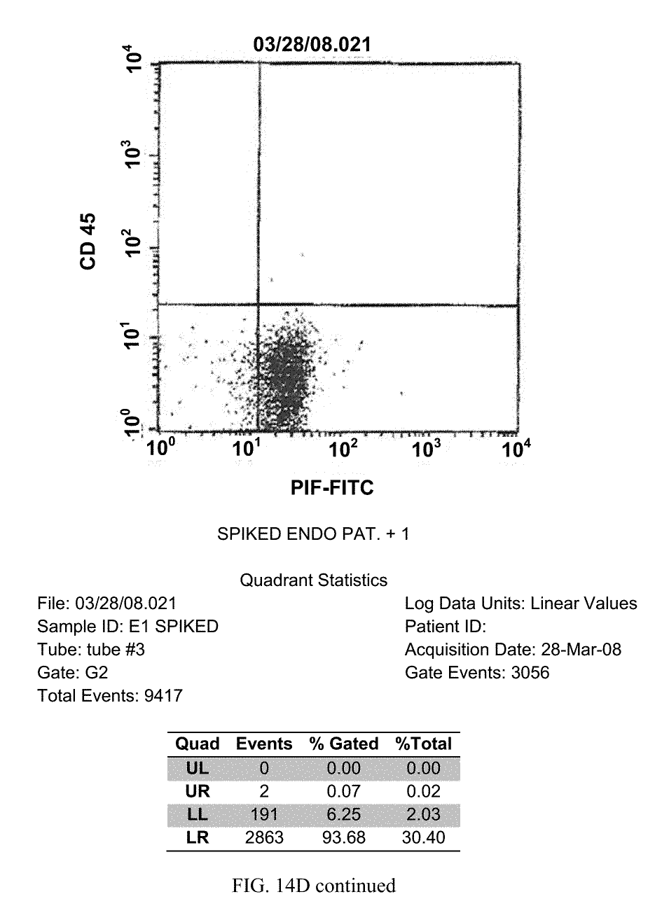

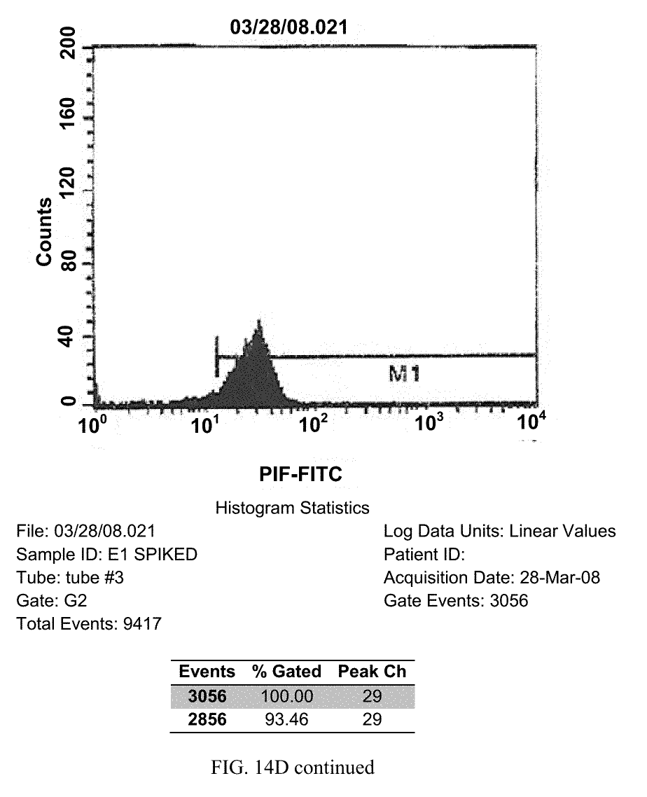

Because endometriosis is an immune disorder, we aimed to determine whether sera from these patients affects proper FITC-PIF interaction (FIGS. 14A-D). We found that binding of PIF to both CD3+ and CD45+ cells is altered in the presence of endometriosis sera (FIGS. 14A and 14B), as compared to control sera (FIGS. 14 C and 14D). Such data provides evidence that PIF binding could provide a sensitive index for determining whether patients have endometriosis, and will serve as a basis to identify which factor(s) could lead to altered binding.

PIF Detection in Early Equine Pregnancy

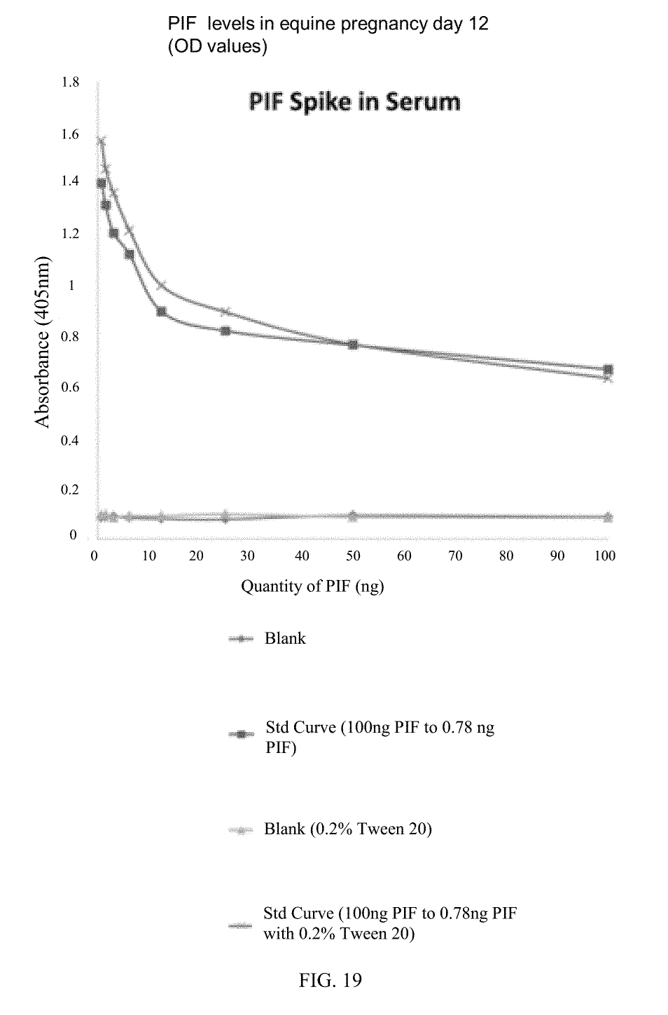

After insemination, mares at day 12 of gestation were tested using PIF-based ELISA using an anti-PIF-based monoclonal antibody based assay with serum samples. PIF-tag lnmol (steriform high avidity)—binds tightly to the plate coated on plate. The plate was blocked with Seablock/Tween and washed. They were premixed with 5 ug/ml Biotin-AntiPIF-Mab+ PIF (0.78 ng-100) ng/ml+serum/buffer 1:4 or buffer and incubated for 1 h. 100 ul of mixture, control and samples were added to the plate. Strepavidin+HRP, was added and incubated for 45 min. Results were read with an ELISA plate reader at 450 nm. The mean levels in the pregnant population (n=19) were compared with samples in non-pregnant patients (n=10). FIG. 19 shows that PIF OD levels are significant in the pregnant as compared with the non-pregnant population, P<0.003. The STD curve also shows demonstrated that the assay is linear.

PIF Binding to Pregnant Mares

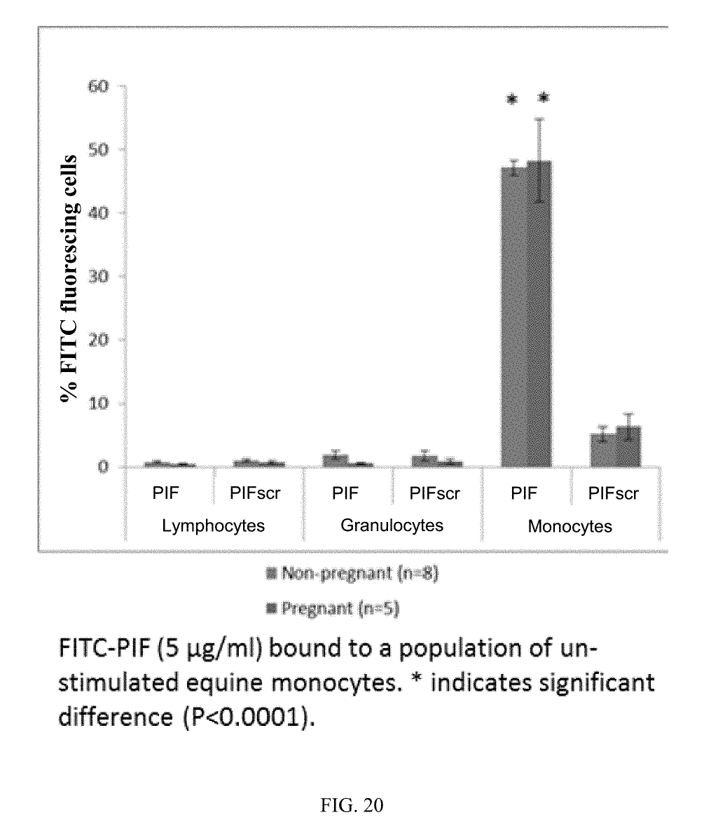

Method: whole blood was collected from jugular vein of pregnant (n=4) and non-pregnant (n=8) mares into lithium heparin vacutainers. Whole blood was layered over histopaque (Sigma) density gradient and red cells were allowed to settle through the gradient, leaving a leukocyte-rich plasma layer above. Cells were washed twice in sterile PBS and any remaining RBCs lysed with 0.16 M ammonium chloride solution. Immune cells were incubated with 1, 5 or 10 (xg/ml FITC-PIF or FITC-PIFscr for 1 hour at room temperature, then washed three times to remove un-bound peptide and fixed for flow cytometry. Cell types were separated based upon their scatter characteristics. Data showed that FITC-binding was most evident in monocytes with minimal binding to other groups was noted. Also no differences were found between pregnant and non-pregnant mares. FIG. 20 and Table 20 show mean+/−SEM in both pregnant and non-pregnant mares, and depicts binding characteristics showing significant differences between FITC-PIF and control, PO0.001.

TABLE 20
FITC-PIF binding to mare immune cells
Pregnant/Non pregnant

| Lymphocytes | | Granulocytes | | Monocytes | |
|---|---|---|---|---|---|
| PIF | PIFscr | PIF | PIFscr | PIF | PIFscr |

Non-pregnant mares

| | | | | | | | |
|---|---|---|---|---|---|---|---|
| 1 ug/ml (n = 8) | Mean | 0.08 | 0.27 | 0.15 | 0.26 | 14.41 | 1.10 |
| | SEM | 0.02 | 0.08 | 0.05 | 0.13 | 2.61 | 0.38 |
| 5 ug/ml (n = 8) | Mean | 0.08 | 0.27 | 0.15 | 0.26 | 14.41 | 1.10 |
| | SEM | | | | | | |
| 10 ug/ml (n = 7) | Mean | 0.02 | 0.08 | 0.05 | 0.13 | 2.61 | 0.38 |
| | SEM | | | | | | |

Pregnant mares

| | | | | | | | |
|---|---|---|---|---|---|---|---|
| 1 ug/ml (n = 4) | Mean | 0.03 | 0.12 | 0.06 | 0.09 | 10.44 | 0.78 |
| | SEM | 0.01 | 0.03 | 0.01 | 0.03 | 2.31 | 0.23 |
| 5 ug/ml(n = 4) | Mean | 0.43 | 0.78 | 0.56 | 0.93 | 48.23 | 6.36 |
| | SEM | 0.08 | 0.21 | 0.10 | 0.36 | 6.51 | 2.05 |
| 10 ug/ml (n = 4) | Mean | 1.38 | 2.84 | 1.94 | 4.51 | 61.77 | 31.07 |
| | SEM | 0.21 | 0.67 | 0.71 | 1.44 | 3.80 | 6.43 |

FITC-PIF selectively bound to a population of naive monocytes (P < 0.0001) regardless of pregnancy status. This effect was not replicated with FITC-PIFscr.

Table 21 below shows a comparison of FITC-PIF binding to non-pregnant CD+/CD25+, CD8+/CD25+, CD4+/CD45+, versus pregnant CD4+/CD45+ binding. Mean +/−SD, 2SD.

TABLE 21

Non Pregnant
CD4+/CD25+
CD25+ 16.1 +/− 1.6 SD, SD2 = 3.2
CD4+ 3.9 +/− 0.2 SD, SD2 = 0.4
CD8+/CD25+
CD25+ 14.8 +/− 1.9 SD, SD2 = 3.8
CD8+ 4.7 +/− 1.3 SD SD2 = 2.6
Not pregnant CD4+/CD45+
CD4+ 9.2 +/− 0.3 SD SD2 = 0.6
CD25+ 20 +/− 4.3 SD SD2 = 4.6
Pregnant CD4+/CD45+
CD4+ 4.6 +/− 2.3 SD SD2 = 4.6
CD25+ 16.9 +/− 5 SD SD2 = 10

FITC-PIF Binding to PBMC Subpopulations in Non-Pregnant and Pregnant Population

The binding of PIF to various T cell populations was examined using 2 color flow cytometry and specific anti-CD4+, CD8+, CD25+, CD45+ antibodies. Results expressed as mean+/-SD as well as SD2.

NFAT1 Assessment: the use of human subject materials adult peripheral blood, involves collection under IRB protocol ‘Hematopoietic Stem Cell Facility’ and was approved by (IRB 09-90-195, University Hospitals of Cleveland). The method to carry out the study is in accordance with the approved guidelines.

Flow Cytometry Studies: non-pregnant infertile and first-trimester pregnant patients at Millenova Immunology Laboratories who were undergoing fertility treatments signed a standard informed consent (CARI, Institute, Chicago). All experiments were performed in accordance with the guidelines and regulations of CARI, Institute, Chicago and with the approval from the Institutional Review Board of the University of Illinois at Chicago in March 2006. The blood was drawn as part of their work-up process with the use of excess specimen without identifiers. We reported on FITC-PIF binding to CD 14+ and CD3+, cells in both pregnant and non-pregnant patients. We examined binding also to CD45+ cells in the same patient population using the anti-CD45 antibody and isotype antibody used as negative control (BD Pharmingen, San Jose, Calif.). The CD45 target is known as a pan leukocyte marker relevant for immune tolerance. In addition, white blood cells or splenocytes were collected from C57BL/6 female mice (aged 8-11 w) and exposed to FITC-PIF at different concentrations for 1 hr on ice. Cells were washed and re-suspended in 1 mL of FACS buffer (Becton-Dickinson, Franklin Lakes, N.J.) and the percentage of FITC-PIF binding cells was measured. To document binding specificity, splenocytes were also exposed to a 100-fold higher concentration of unlabeled PIF which was followed by flow cytometry analysis. Identification of the cell type associated with PIF bound to circulating murine immune cells was tested. Immune cells were collected following mouse sacrifice. Collected cells were incubated with FITC-PIF, (12.5-50 ug/ml,) plus anti-CD45 (BD Pharmingen, San Jose, Calif.). Isotype controls served as negative controls. Two-color staining was done using conventional techniques. Fluorescence measurements (20,000-50,000 gated events per sample) were performed in a Coulter® Epics® XL™ Flow Cytometer using System II software for data acquisition and analysis (Beckman Coulter, Inc., Miami, Fla.).

Statistical Analysis: protein probabilities were analyzed using Protein Prophet algorithm software. Protein target clustering and interaction was determined using String version 9.1 software. Gene pathway analysis was performed using the Ingenuity Systems Inc. (Redwood Calif.) software, ranking by greatest number of genes in a given pathway.

PIF Down-Regulates NFAT1 Expression in CD4+ Cells

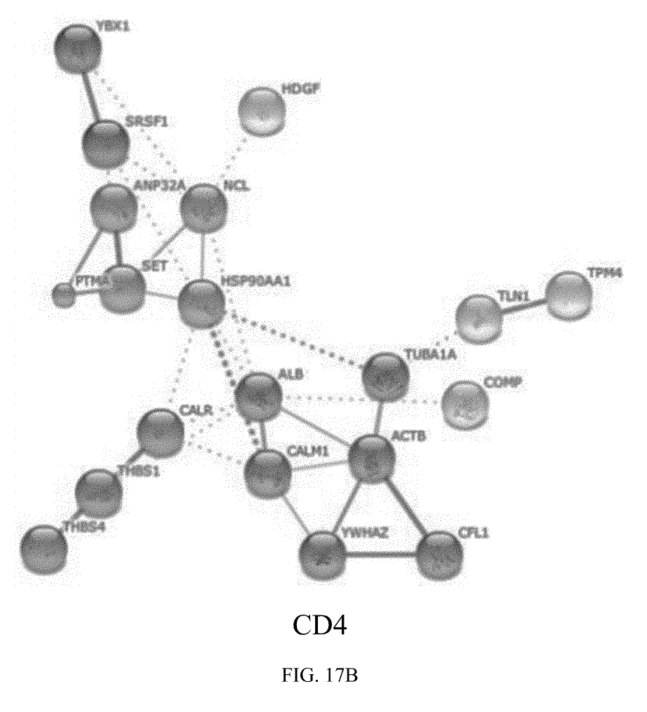

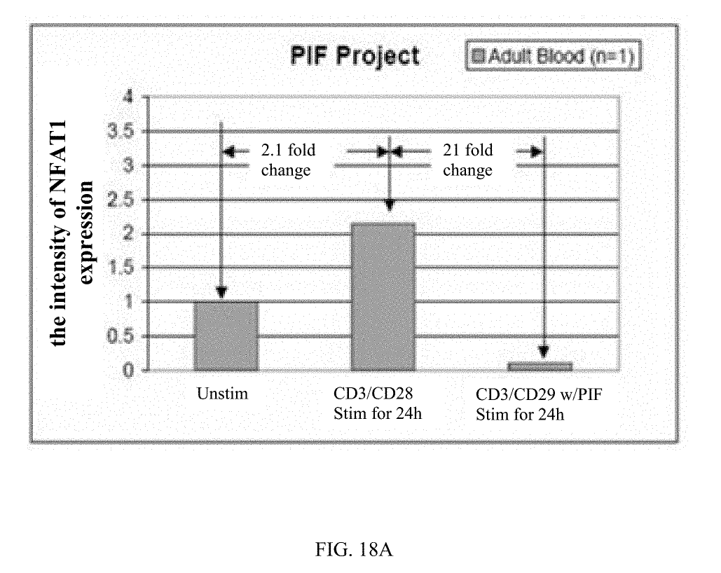

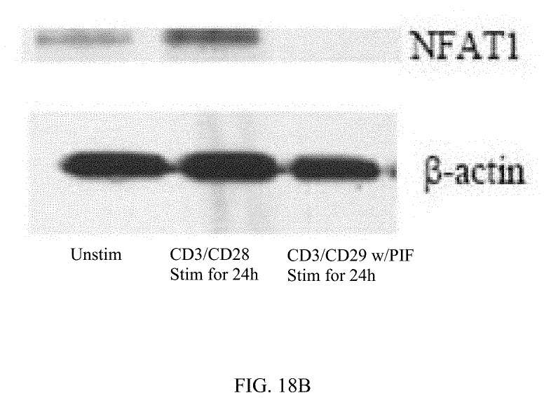

We reported that PIF does not affect early Ca++ flux in PBMC. However, previous gene data and current cluster analysis documented that PIF targets a number of calcium regulatory proteins including calmodulin and calreticulin. NFAT1 is a down-stream target and it regulates IL2 secretion which PIF was already demonstrated to regulate in PBMC. Therefore, to link this signaling pathway PIF effect on isolated CD4+ cells (>95% purity) stimulated by anti-CD3/CD28 antibody was determined evaluating NFAT1 expression (Western blot). The use of human subject materials adult peripheral blood, involves collection under IRB protocol ‘Hematopoietic Stem Cell Facility’ was approved by (IRB 09-90-195, University Hospitals of Cleveland). The method to carry out the study is in accordance with the approved guidelines. Blood was obtained from a healthy human donor, purified via ficoll-plaque PBMC separation followed by CD14−/4+ selection by MACS. Cells were cultured for 24 hours in RPMI+10% FBS+1% L-glutamine (unstimulated) or with liig/mL adherent anti-CD3 with 5 ug/mL soluble anti-CD28 testing the PIF effect on NFAT1 (NFATc2) expression. Protein extracts equivalent to 3×10 5 CD4+cells were loaded per lane and analyzed by Western blotting with a combination of anti-NFAT1 (Transduction laboratories) and anti-(3-actin antibodies (Invitrogen). FIG. 18A is a graphic representation of the effect of co-activation on NFAT1 expression and the effect of PIF on the induced cells. FIG. 18B is a Western blot mean quantification of relative NFAT1 expression, normalized for each lane with the p-actin band and the intensity of each NFAT1 band calculated as relative percentage of the most intense NFAT1 band on each gel. The most intense band was set arbitrarily at 100% and relative percentages were then averaged and graphed. Data showed that following co-activation NFAT1 increased 1.7 fold. However, the addition of PIF to the culture has led to a major 27-fold decrease in the expression. This linked the identified protein target with the downstream transcription factor regulation.

FITC-PIF Binds to Circulating CD45+ Immune Cells

To determine which immune cell lineage PIF directly targets in vivo, under unstimulated FITC-PIF interaction with isolated splenocytes and white blood cells was examined using flow cytometry. Increased PIF concentrations led to increased PIF binding with the ligands. To document PIF binding specificity to splenocytes FITC-PIF exposed cells were added to +/−100-fold concentration of unlabeled PIF. Using flow cytometry data showed that in presence of PIF the binding decreased >95%—thereby demonstrating binding specificity. In order to determine which cell type is involved in interaction with PIF, the binding to circulating CD45+ cells in healthy mice was examined. These cells are considered pan leukocyte signaling markers. We found, using two color flow cytometry, that FITC-PIF (12.5-50 ( μg/ml) incubated with isolated circulating mouse white blood cells binds up to 25% of those cells.

FITC-PIF Binding to CD45+ Cells is Decreased During Pregnancy

We reported that FITC-PIF binding to CD 14+ cells is maximal in non-pregnant women and therefore it is unchanged when tested during pregnancy. In contrast the binding to CD3+ cells is low prior to but it is significantly increased during pregnancy. In the same patients as reported we also compared the binding to CD45+ cells. The data showed (n=4/group) that the binding of FITC-PIF to PBMC analyzed by flow cytometry in pregnant subjects was significantly decreased as compared with non-pregnant patients Mean+/−SD (27%+/−6.1 vs 17.25%+/1.7), P<0.01. This data further documents that PIF binding is dynamic, varying with the functional status (i.e. pregnancy) of the subject.

These data integrate both the systemic and specific organ directed PIF targeting. The reduced FITC-PIF binding to CD45+ cells in pregnant subjects may be related to the known tolerance promoting effect of PIF. CD45-ligation promotes T regulatory cells-dendritic cells interaction through increased NFAT1 expression regulated by PIF. PIF interaction with both spleen and immune cells in vitro confirmed binding and specificity of interaction. This provides important insight into the overall protection which is observed using PIF in preclinical models, and reflects a pharmacodynamic rather than a pharmacokinetic effect. These lead to ongoing clinical translation studies. Collectively, we demonstrate PIF's direct, specific and synergistic targeting of naive immune cells involved in protection, immune activation and cell structure. We furthermore confirm that PIF targets systemic immunity coupled with rapid clearance, in vivo. Consequently, PIF is currently being translated into treatment of patients with immune disorders.

PIF Effect on Circulating Cytokines During Murine Pregnancy

Effect on normal mice is compared with LPS treated and LPS+PIF at day 14 of pregnancy in Table 22 below:

TABLE 22

| MEDIA | Sera | | | |
|---|---|---|---|---|
| (pg/ml) | CTR | PIF | LPS | LPS + PIF |
| TNFalfa | 6.6 ± 0.4 | 5.6 ± 1.1 | 8.6 ± 0.9* | 7.7 ± 1.0 |
| IFN gamma | 3.16 ± 0.2 | 3.39 ± 1.4 | 16.6 ± 1.6* | 5.92 ± 1.1§ |
| IL1 beta | 3.4 ± 1 | 2.5 ± 1.1 | 5.2 ± 1.3* | 4 ± 1.3 |
| IL18 | 266 ± 65 | 150 ± 25* | 369 ± 26* | 268 ± 64§ |
| GMCSF | 6.4 ± 1 | 5.3 ± 0.7 | 11.06 ± 1.6* | 8.2 ± 1.5§ |
| GRO | 15.26 ± 1.8 | 18.06 ± 2.2 | 24.9 ± 2.9* | 17.07 ± 1.2§ |
| IL-4 | 2.53 ± 0.48 | 1.74 ± 0.9 | 7.72 ± 0.2* | 3.95 ± 0.3*§ |
| IL-5 | 3.98 ± 0.5 | 2.66 ± 0.4* | 13.7 ± 0.11* | 6.8 ± 0.5*§ |
| IL12p70 | 1.36 ± 0.49 | 0.94 ± 0.7* | 3.14 ± 1.73* | 2.07 ± 1.2*§ |
| IL17a | 5.52 ± 0.06 | 4 ± 0.7 | 13.6 ± 0.54* | 10.4 ± 0.12* |
| IL22 | 35.89 ± 2.1 | 15.9 ± 27* | 47.3 ± 5.6* | 36.2 ± 2.6§ |
| IL23 | 36.29 ± 8.6 | 21.6 ± 2.3* | 66.9 ± 3.2* | 50.9 ± 5.2 |
| IL27 | 22.7 ± 1.1 | 13.4 ± 1.08* | 54.5 ± 2.7* | 38.7 ± 3.1§ |
| MCP1 | 47.04 ± 3.3 | 34.1 ± 2.7* | 49.01 ± 1.9 | 41.8 ± 3.9 |
| MIP 1 beta | 3.95 ± 0.3 | 1.9 ± 0.7* | 6.4 ± 0.4* | 3.6 ± 0.8§ |

PIF effect on cytokines levels in the placenta comparison to control as well as to LPS treated and PIF effect on LPS treated mice is shown in Table 23 below:

TABLE 23

| | PLACENTA | | | |
|---|---|---|---|---|
| | CTR | PIF | LPS | LPS + PIF |
| TNFalfa | 9.6 ± 1 | 8.2 ± 0.9 | 14.4 ± 0.8* | 12.1 ± 1*§ |
| IL1 beta | 4.7 ± 0.5 | 2.8 ± 0.4* | 5.2 ± 0.3 | 4.5 ± 1.2 |
| IL18 | 203 ± 23.2 | 290 ± 21.6* | 335 ± 20.4* | 223 ± 24.4 |
| GRO | 1060 ± 159 | 1255 ± 218* | 1686 ± 401* | 1335 ± 550 |
| IL5 | 2.3 ± 0.73 | 4.41 ± 0.5* | 5.2 ± 1.2* | 4.05 ± 0.9 |
| IL12p70 | 0.13 ± 0.06 | 0.82 ± 0.7* | 0.35 ± 0.54 | 0.2 ± 0.12 |
| IL23 | 279.7 ± 58.8 | 96.7 ± 28.3* | 208.7 ± 48.7 | 204.7 ± 61.8 |

§P < 0.05 vs LPS;
*P < 0.05 vs CTR

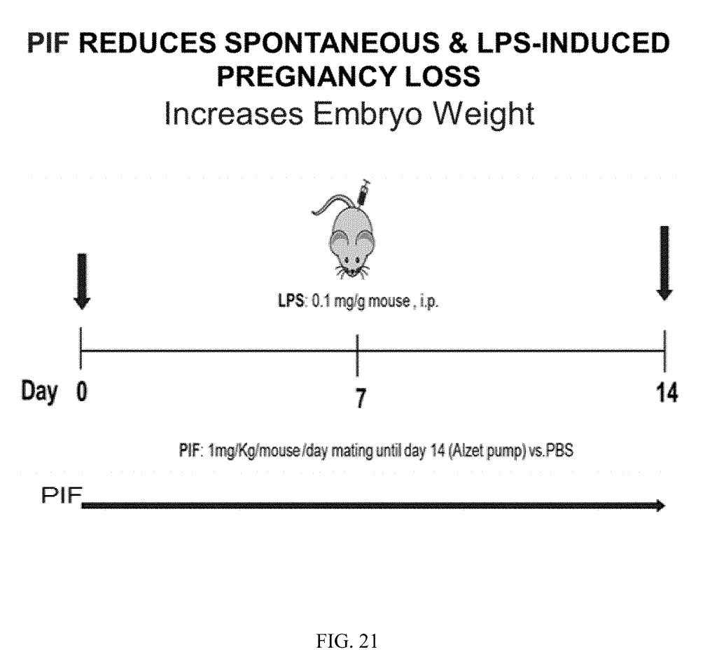

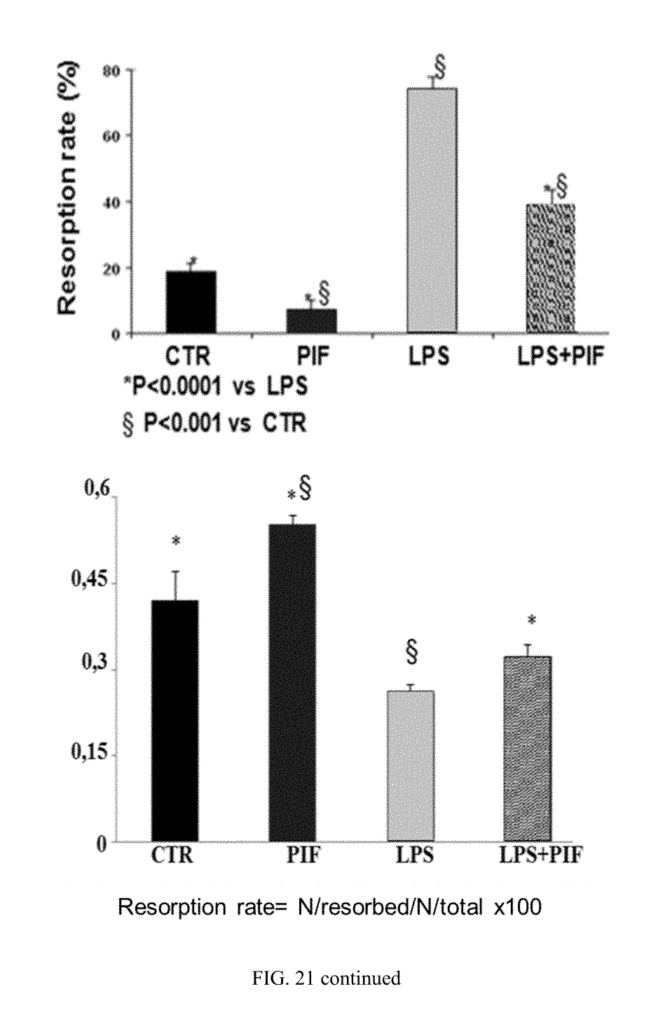

This data indicates that PIF regulates several cytokines both in serum and in the placenta. Of note, in order to improve pregnancy PIF also promotes fetal weight, as well as reduces the rate of spontaneous LPS-induced pregnancy loss, as shown in FIG. 21.

1. A method of identifying a female subject with or likely to suffer from recurrent pregnancy loss (RPL) due to immune dysregulation comprising:
exposing an effective amount of preimplantation factor (PIF) or an analog thereof to a sample from the subject comprising one or a plurality of immune cells;
examining a binding event between the one or among a plurality of immune cells of the subject and PIF or an analog thereof; and
identifying the female subject as suffering from or likely to suffer from RPL due to immune dysregulation if there is a significant change in binding between PIF or analog thereof and the one or a plurality of immune cells. as compared to binding to one or plurality of immune cells in a reference.

2-3. (canceled)

4. The method of claim 1 further comprising isolating a sample from the subject prior to exposing the sample to PIF or an analog thereof.

5. The method of claim 1, further comprising immobilizing PIF or an analog thereof to a solid support prior to exposing the PIF or analog thereof to one or a plurality of immune cells, wherein the solid support is chosen from a chip, a column, a plate or a multiwell plate.

6. (canceled)

7. The method of claim 1, wherein the step of examining a binding event comprises observing, quantifying and/or detecting the association between PIF or an analog thereof and one or a plurality of immune cells.

8. The method of claim 1, wherein the step of examining a binding event comprises observing, quantifying and/or detecting an amount of expression of one or a plurality of cytokines by the one or plurality of immune cells.

9. The method of claim 1, wherein the step of examining a binding event comprises observing, quantifying and/or detecting a number of immune cells that bind to PIF or an analog thereof, wherein the one or plurality of immune cells comprise one or a combination of CD3+ cell, CD4+ cells, CD14+ cells, CD45+ cells, dendritic cells, peripheral blood mononuclear cells.

10-12. (canceled)

13. The method of claim 1, wherein the significant change comprises quantifying a decrease in said PIF binding to CD 14+ and/or dendritic cells.

14. The method of claim 1, wherein the significant change comprises quantifying an increase in said PIF binding to CD4+, CD8+, and/or natural killer (NK) cells.

15. The method of claim 1, wherein the PIF or analog thereof comprises one or more fluorescein isothiocyanate (FITC) label, and wherein a binding event is measured by quantifying and/or detecting the level of fluorescence by flow cytometry.

16. (canceled)

17. The method of claim 1, wherein the significant change comprises one or a combination of a reduction of PIF or an analog thereof binding to dendritic cells, an increase of PIF or an analog thereof binding to CD 14+ cells, and an increase of PIF binding to CD4+ cells.

18-34. (canceled)

35. A method of identifying a female subject with or likely to suffer from endometriosis comprising:
exposing an effective amount of preimplantation factor (PIF) or an analog thereof to a sample from the subject comprising one or a plurality of immune cells;
examining a binding event between the one or among a plurality of immune cells of the subject and PIF or an analog thereof; and
identifying the female subject as suffering from or likely to suffer from endometriosis is if there is a significant change in binding between PIF or an analog thereof and one or plurality of immune cells as compared to binding to one or a plurality of immune cells in a reference.

36-37. (canceled)

38. The method of claim 35 further comprising isolating a sample from the subject prior to exposing the sample to PIF or an analog thereof.

39. The method of claim 35, further comprising immobilizing PIF or an analog thereof to a solid support prior to exposing the PIF or analog thereof to one or a plurality of immune cells, wherein the solid support is chosen from a chip, a column, a plate or a multiwall plate.

40. (canceled)

41. The method of claim 35, wherein the step of examining a binding event comprises observing, quantifying and/or detecting the association between PIF or an analog thereof and one or a plurality of immune cells.

42. The method of claims 35, wherein the step of examining a binding event comprises observing, quantifying and/or detecting an amount of expression of one or a plurality of cytokines by the one or plurality of immune cells.

43. The method of claim 41, wherein the step of examining a binding event comprises observing, quantifying and/or detecting a number of immune cells that bind to PIF or an analog thereof, wherein the one or plurality of immune cells comprise one or a combination of CD3+ cell, CD4+ cells, CD14+ cells, CD45+ cells, dendritic cells, peripheral blood mononuclear cells.

44-46. (canceled)

47. The method of claim 35, wherein the significant change comprises quantifying a decrease in said PIF binding to CD 14+ and/or dendritic cells.

48. The method of claim 35, wherein the significant change comprises quantifying an increase in said PIF binding to CD4+, CD8+, and/or natural killer (NK) cells.

49. The method of claim 35, wherein the PIF or analog thereof comprises one or more fluorescein isothiocyanate (FITC) label, and wherein a binding event is measured by quantifying and/or detecting the level of fluorescence by flow cytometry.

50. (canceled)

51. The method of claim 35, wherein the significant change comprises one or a combination of an increase of PIF or an analog thereof binding to CD3+ cells and an increase of PIF binding to CD45+ cells.

52-68. (canceled)

69. A method of detecting a level of immune dysregulation sufficient to cause recurrent pregnancy loss (RPL) or endometriosis comprising:
exposing a sample from a subject preimplantation factor (PIF) or an analog thereof that is about 75% homologous to any PIF amino acid selected from: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, mimetics thereof, and combinations thereof; quantifying a number of immune cells that bind to PIF or the analog thereof; comparing the number of immune cells bound to PIF or an analog thereof to a number of immune cells that bind to PIF or the analog thereof from a sample of subject that does not have immune dysregulation sufficient to cause RPL or endometriosis; and
classifying the subject as having immune dysregulation sufficient to cause RPL or endometriosis if the number of immune cells bound to PIF or the analog thereof is from about fifteen to about forty percent greater than the number of immune cells bound to PIF or the analog thereof from a sample of subject that does not have immune dysregulation sufficient to cause RPL or endometriosis.

70. The method of claim 69, wherein the PIF or or analog thereof is immobilized to a solid support.

71. The method of claim 69, wherein the method further comprises creating a binding profile of the subject.

72. A method of treating a subject having a level of immune dysregulation sufficient to cause RPL or endometriosis comprising: detecting the presence, absence, or quantity of one or more of immune cells in a sample from the subject; diagnosing the subject as having a level of immune dysregulation sufficient to cause RPL if the number of immune cells in the sample from the subject is about twenty percent greater than a reference number of immune cells; and treating the subject by administering an effective amount of an immunomodulating agent.

73-77. (canceled)

78. A method of detecting a level of immune dysregulation of a subject comprising:
detecting or quantifying a number of immune cells that bind to PIF or an analog thereof that is about 75% homologous to any PIF amino acid selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, mimetics thereof, and combinations thereof;
comparing the number of immune cells bound to PIF or the analog thereof to a number of immune cells that bind to PIF from a sample of subject that does not have immune dysregulation; and
classifying the subject as having immune dysregulation if the number of immune cells bound to PIF or the analog thereof is about twenty percent greater than the number of immune cells bound to PIF or the analog thereof from a sample of subject that does not have known immune dysregulation.

79-82. (canceled)

83. The method of claim 69, wherein the immune cells comprise one or a combination of: CD14+, CD4+, or CD8+ cells.

84. The method of claim 71, wherein the step of creating a binding profile comprises correlating a level of immune dysregulation with the quantity of one or a combination of: the binding affinity of 14-3-13 eta bound to PIF or the analog thereof, the binding affinity of 14-3-3 eta bound to PIF or the analog thereof, the binding affinity of Myosin 9 bound to PIF or the analog thereof, the binding affinity of Thymosin-al bound to PIF or the analog thereof, and the number of CD8+ cells from CD4+, CD8+, or CD14+ cells bound to PIF or the analog thereof comprises calculating protein interactions, including direct and indirect associations, using a database of known and predicted protein interactions.

85-87. (canceled)

88. The method of any of claims 69, wherein the number of immune cells bound to PIF or the analog thereof from a reference or control is about twenty percent less than the number of immune cells bound to PIF or the analog thereof from a sample of the subject.

89-90. (canceled)
